(19) United States
(12) Patent Application Publication
(10) Pub. No.: US 2024/0100188 A1
(43) Pub. Date: Mar. 28, 2024

(54) REGULATORY SYSTEM FOR EXPRESSION OF A GENE OF INTEREST IN A TARGET CELL AND METHOD OF USE THEREOF

(71) Applicant: ICAHN SCHOOL OF MEDICINE AT MOUNT SINAI, New York, NY (US)

(72) Inventors: Lior ZANGI, New York, NY (US); Keerat KAUR, New York, NY (US); Magdalena ZAK, NewYork, NY (US)

(73) Assignee: ICAHN SCHOOL OF MEDICINE AT MOUNT SINAI, New York, NY (US)

(21) Appl. No.: 18/349,494

(22) Filed: Jul. 10, 2023

Related U.S. Application Data
(60) Provisional application No. 63/359,989, filed on Jul. 11, 2022.

(51) Int. Cl. A61K 48/00; A61P 9/10; C12N 9/22; C12N 15/113; C12N 15/90

(52) U.S. Cl. CPC A61K 48/0058; A61P 9/10; C12N 9/22; C12N 15/113; C12N 15/907; C12N 2310/3341; C12N 2310/335; C12N 2840/007; C12N 2840/105

(57) ABSTRACT

Provided is a system for regulating expression of a gene of interest in a target cell, including a recombinant first RNA molecule with (i) a coding sequence for a translation-suppressor protein and (ii) a first microRNA (miR) recognition element in its 3′ UTR, wherein the first miR recognition element recognizes a first miR and binding of a first miR to the first miR recognition element reduces translation of the translation suppressor, and a recombinant second RNA molecule, with (i) a coding sequence for the gene of interest, (ii) a recognition sequence for the translation-suppressor, wherein binding of the translation-suppressor to the recognition sequence for the translation-suppressor reduces translation of the gene of interest, and, optionally, (iii) a second miR recognition element in its 3′ UTR, wherein the second miR recognition element recognizes one or more second miR and binding of one or more of the one or more second miR to the second miR recognition element reduces translation of the gene of interest. Also provided are methods of using the system.

### CROSS-REFERENCE TO RELATED APPLICATIONS

This application claims benefit of priority to U.S. Provisional Patent Application 63/359,989, filed Jul. 11, 2022, the entire content of which is hereby incorporated herein by reference in its entirety.

### SEQUENCE LISTING

The instant application contains an electronic sequence listing. The contents of the electronic sequence listing H2715538.xml; Size: 61,503 bytes; and Date of Creation: Jul. 10, 2023, is herein incorporated by reference in its entirety.

### BACKGROUND

Modifying cellular function by delivery of genes of interest for expression in cells of a given, pre-determined tissue or cellular phenotype or genotype is an attractive method for various therapeutic, diagnostic, experimental, or other purposes. Delivery of polynucleotides such as RNA into cells is an example of such a method. RNA may be injected “naked” into tissues for cellular uptake or packaged in nanoparticles of various composition, permitting cellular uptake in various tissues following systemic injection. However, in some cases, it is desirable to drive expression of a gene of interest in cells of a given, pre-determined tissue or cellular phenotype of genotype (referred to herein as a “target cell”) without also driving expression in cells of a different tissue or cellular phenotype of genotype (referred to herein as “off-target cells”).

For example, a gene product such as a protein of a gene of interest may have beneficial effects when its expression is driven in a target cell type whereas it may have deleterious effects if its expression is also driven to strongly in off-target cells, or if expression in off-target cells is too high. Difficulties in, for example, limiting RNA uptake to target cells as opposed to off-target cells makes preferential or exclusive expression in target cells difficult to obtain following systemic treatment with RNA molecules. What is needed is a system of RNA polynucleotides permitting preferential, predominant, or in some cases even exclusive expression of a gene of interest in a target cell and no, low, undetectable, or low expression, relatively, in off-target cells, following application thereof to tissue cells, in vivo, in vitro, or ex vivo. The present disclosure is directed to overcoming these and other deficiencies in the art.

### SUMMARY

In an aspect, provided is an expression regulatory system for expression of a gene of interest in a target cell, comprising a recombinant first RNA molecule, comprising (i) a coding sequence for a translation-suppressor protein and (ii) a first microRNA (miR) recognition element in its 3′ UTR, wherein the first miR recognition element recognizes one or more first miR and binding of one or more of the one or more first miR to the first miR recognition element reduces translation of the translation suppressor, and a recombinant second RNA molecule, comprising (i) a coding sequence for the gene of interest, (ii) a recognition sequence for the translation-suppressor, wherein binding of the translation-suppressor to the recognition sequence for the translation-suppressor reduces translation of the gene of interest, and (iii) a second miR recognition element in its 3′ UTR, wherein the second miR recognition element recognizes one or more second miR and binding of one or more of the one or more second miR to the second miR recognition element reduces translation of the gene of interest.

In an example, one or more of the one or more first miR is expressed in the target cell and one or more of the one or more second miR is expressed in an off-target cell. In another example, one or both of the first RNA molecule and the second RNA molecule comprises one or more of a modified ribonucleotide and an anti-reverse cap analog. In still another example, one or both of the first RNA molecule and the second RNA molecule comprises one or more modified ribonucleotide and the one or more modified ribonucleotide is independently selected from pseudouridine and cytidine. In a further example, wherein one or both of the first RNA molecule and the second RNA molecule comprises an anti-reverse cap analog and the anti-reverse cap analog is selected from 3′-O-Me-m7G(5′)ppp(5′)G cap. In still a further example, the translation-suppressor and the recognition sequence for the translation-suppressor comprise, respectively, Cas6 and a Cas6 recognition site or L7Ae and a k-turn motif.

In another example, the target cell is a heart tissue cell, a lung tissue cell, a liver tissue cell, a spleen tissue cell, or a tumor cell. In still another example, the off-target cell is selected from one or more of a heart tissue cell, a lung tissue cell, a liver tissue cell, a spleen tissue cell, or a tumor cell. In yet another example, the target cell is a tumor cell. In a further example, the target cell is a breast tumor cell. In still a further example, the off-target cell is selected from one or more of a heart tissue cell, a lung tissue cell, a liver tissue cell, a spleen tissue cell, or a tumor cell. In yet a further example, the gene of interest encodes a protein and the protein is selected from an anti-apoptotic protein, a pro-apoptotic protein, a cell cycle-inducer protein, and a cell-cycle arrest protein.

In another example, the gene of interest encodes a pro-apoptotic protein. In still another example, the gene of interest encodes an anti-apoptotic protein. In yet another example, the gene of interest encodes a cell cycle-inducer protein and the cell cycle-inducer protein is selected from Lin28, Pkm2 and Cyclin D2. In a further example, the gene of interest encodes a cell cycle-arrest protein. In still a further example, the gene of interest encodes an acid ceramidase a type 2 phosphatidylinositol-5-phosphate 4-kinase gamma a Lin28, a Pkm2, a Cyclin D2, a p53 protein, a Herpes Simplex Virus type 1 thymidine kinase, a deltex protein, an ElA protein, a cystatin SA protein, a cystatin E/M protein, or a caspase 9 protein. In yet a further example, the gene of interest encodes a protein and the protein is selected from an antibody, an anti-angiogenic protein, and an angiogenic protein.

In another example, the gene of interest encodes a marker protein. In still another example, the gene of interest encodes a marker protein and the marker protein is selected from a green fluorescence protein, inactive human CD25, inactive mouse CD25, beta-galactosidase, and luciferase. In yet another example, the gene of interest encodes a fluorescent protein. In a further example, the gene of interest encodes a fluorescent protein and the fluorescent protein is selected from a green fluorescent protein, a yellow fluorescent protein, mCherry, and tdTomato.

Another example further comprises a nanoparticle, wherein the nanoparticle comprises the first RNA molecule and the second RNA molecule. In still another example, the nanoparticle comprises any one or more of a liposome nanoparticle, a gold nanoparticle, an iron nanoparticle, a poly lactic-co-glycolic acid nanoparticle, and a viral vector. In yet another example, the nanoparticle comprises a positive charge, a negative charge, or a neutral charge.

In another example, the second miR recognition element includes one or more of a miR-195a recognition element, a miR-200c recognition element, a miR-Let7f recognition element, a miR-143 recognition element, a miR-222 recognition element, a miR-142a recognition element, a miR-122 recognition element, a miR-146a recognition element, a miR-34c recognition element, a miR-17 recognition element, a miR-125 recognition element, a miR-26a2 recognition element, a miR-92a recognition element, a miR-20a recognition element, and a miR-486a recognition element, a miR-146a recognition element, and any combination of two or more of the foregoing. In still another example, the first miR recognition element comprises one or both of a miR-1 recognition element and a miR-208 recognition element. In yet another example, the second miR recognition element comprises one or both of a miR-143 recognition element and a miR-146a recognition element. In a further example, the first miR recognition element comprises one or both of a miR-1 recognition element and a miR-208 recognition element and the second miR recognition element comprises one or both of a miR-143 recognition element and a miR-146a recognition element. In still a further example, the translation-suppressor and the recognition sequence for the translation-suppressor comprise, respectively, Cas6 and a Cas6 recognition site.

In another example, the target cell is a heart tissue cell. In still another example, the one or more off-target cell is selected from one or more of a heart tissue cell, a lung tissue cell, a liver tissue cell, and a spleen tissue cell. In yet another example, the target cell is a cardiomyocyte. In a further example, one or more of the one or more off-target cells is a non-cardiomyocyte heart tissue cell. Still a further example further comprises a nanoparticle, wherein the nanoparticle comprises the first RNA molecule, the second RNA molecule, and a positive charge. In yet a further example, the gene of interest encodes an acid ceramidase a type 2 phosphatidylinositol-5-phosphate 4-kinase gamma a Lin28, a Pkm2, or a Cyclin D2.

In another example, the first miR recognition element comprises one or more of a miR-155 recognition element, a miR10b recognition element, a miR181a recognition element, and miR-181b recognition element. In still another example, the second miR recognition element comprises one or more of a miR143 recognition element and a miR122 recognition element. In yet another example, the first miR recognition element comprises one or more of a miR-155 recognition element, a miR10b recognition element, a miR18-la recognition element, and miR-181b recognition element, and the second miR recognition element comprises one or more of a miR143 recognition element and a miR122 recognition element. In a further example, the first miR recognition element comprises a miR-155 recognition element. In still a further example, the second miR recognition element comprises a miR122 recognition element. In yet a further example, the first miR recognition element comprises a miR-155 recognition element and the second miR recognition element comprises a miR122 recognition element. In another further example, the translation-suppressor and the recognition sequence for the translation-suppressor comprise, respectively, Cas6 and a Cas6 recognition site.

In another example, the target cell comprises a tumor cell. In still another example, the tumor cell comprises a breast tumor cell. In yet another example, one or more of the one or more off-target cells is selected from a heart tissue cell, a lung tissue cell, a liver tissue cell, and a spleen tissue cell. Yet another example further comprises a nanoparticle, wherein the nanoparticle comprises the first RNA molecule, the second RNA molecule, and a neutral charge. In a further example, the gene of interest encodes a p53 protein, a Herpes Simplex Virus type 1 thymidine kinase, a deltex protein, an E1A protein, a cystatin SA protein, a cystatin E/M protein, or a caspase 9 protein. In still a further example, the gene of interest encodes a tumor-suppressor protein.

In another aspect, provided is a method, comprising administering the expression regulatory system to a subject, wherein the subject suffered a myocardial infarction or suffers from heart failure. In another example, the expression regulatory system further comprises a nanoparticle and the nanoparticle comprises the first RNA molecule, the second RNA molecule, and a positive charge. In still another example, administering comprises administering the nanoparticle intravenously. In yet another example, the administering comprises administering by intramyocardial injection. In a further example, the administering comprises administering two or more times. In still a further example, the administering comprises stimulating proliferation of cardiomyocytes.

In another example, the second miR recognition element includes one or more of a miR-195a recognition element, a miR-200c recognition element, a miR-Let7f recognition element, a miR-143 recognition element, a miR-222 recognition element, a miR-142a recognition element, a miR-122 recognition element, a miR-146a recognition element, a miR-34c recognition element, a miR-17 recognition element, a miR-125 recognition element, a miR-26a2 recognition element, a miR-92a recognition element, a miR-20a recognition element, and a miR-486a recognition element, a miR-146a recognition element, and any combination of two or more of the foregoing. In still another example, the first miR recognition element comprises one or both of a miR-1 recognition element and a miR-208 recognition element. In yet another example, the second miR recognition element comprises one or both of a miR-143 recognition element and a miR-146a recognition element. In a further example, the first miR recognition element comprises one or both of a miR-1 recognition element and a miR-208 recognition element and the second miR recognition element comprises one or both of a miR-143 recognition element and a miR-146a recognition element. In still a further example, the first miR recognition element comprises one or both of a miR-1 recognition element and a miR-208 recognition element and the second miR recognition element comprises one or both of a miR-143 recognition element and a miR-146a recognition element.

In another example, the target cell is a heart tissue cell. In still another example, he one or more off-target cell is selected from one or more of a heart tissue cell, a lung tissue cell, a liver tissue cell, and a spleen tissue cell. In yet another example, the target cell is a cardiomyocyte. In a further example, one or more of the one or more off-target cells is a non-cardiomyocyte heart tissue cell.

Another example, the expression regulatory system further comprises a nanoparticle, wherein the nanoparticle comprises the first RNA molecule, the second RNA molecule, and a positive charge. In still another example, the gene of interest encodes an acid ceramidase, a type 2 phosphatidylinositol-5-phosphate 4-kinase gamma a Lin28, a Pkm2, or a Cyclin D2.

In still another aspect, provided is a method, comprising administering the expression regulatory system to a subject, wherein the subject suffers from cancer. In an example, the subject suffers from breast cancer. In another example, the expression regulatory system further comprises a nanoparticle and the nanoparticle comprises the first RNA molecule, the second RNA molecule, and a neutral charge. In still another example, administering comprises administering the nanoparticle intravenously. In yet another example, the administering comprises administering by intratumoral injection. In a further example, the administering comprises administering two or more times. In still a further example, the administering comprises inhibiting tumor growth.

In another example, the second miR recognition element includes one or more of a miR-195a recognition element, a miR-200c recognition element, a miR-Let7f recognition element, a miR-143 recognition element, a miR-222 recognition element, a miR-142a recognition element, a miR-122 recognition element, a miR-146a recognition element, a miR-34c recognition element, a miR-17 recognition element, a miR-125 recognition element, a miR-26a2 recognition element, a miR-92a recognition element, a miR-20a recognition element, and a miR-486a recognition element, a miR-146a recognition element, and any combination of two or more of the foregoing. In still another example, the first miR recognition element comprises one or more of a miR-155 recognition element, a miR10b recognition element, a miR18-la recognition element, and miR-181b recognition element. In yet another example, the second miR recognition element comprises one or more of a miR143 recognition element and a miR122 recognition element. In a further example, the first miR recognition element comprises one or more of a miR-155 recognition element, a miR10b recognition element, a miR181a recognition element, and miR-181b recognition element, and the second miR recognition element comprises one or more of a miR143 recognition element and a miR122 recognition element. In still a further example, the first miR recognition element comprises a miR-155 recognition element. In yet a further example, the second miR recognition element comprises a miR122 recognition element.

In another example, the first miR recognition element comprises a miR-155 recognition element and the second miR recognition element comprises a miR122 recognition element. In still a further example, the first miR recognition element comprises a miR-155 recognition element and the second miR recognition element comprises a miR122 recognition element In yet another example, the target cell comprises a tumor cell. In a further example, the tumor cell comprises a breast tumor cell. In still a further example, one or more of the one or more off-target cells is selected from a heart tissue cell, a lung tissue cell, a liver tissue cell, and a spleen tissue cell.

In another example, the expression regulatory system further comprises a nanoparticle, wherein the nanoparticle comprises the first RNA molecule, the second RNA molecule, and a neutral charge. In still another example, the gene of interest encodes a p53 protein, a Herpes Simplex Virus type 1 thymidine kinase, a deltex protein, an E1A protein, a cystatin SA protein, a cystatin E/M protein, or a caspase 9 protein. In yet another example, the gene of interest encodes a tumor-suppressor protein.

In yet another aspect, provided is an expression regulatory system for expression of a gene of interest in a target cell, comprising a recombinant first RNA molecule, comprising (i) a coding sequence for Cas6 and (ii) a first microRNA (miR) recognition element in its 3′ UTR, wherein the first miR recognition element recognizes one or more first miR and binding of one or more of the one or more first miR to the first miR recognition element reduces translation of the translation suppressor, and a recombinant second RNA molecule, comprising (i) a coding sequence for the gene of interest, (ii) a Cas6 recognition sequence, wherein binding of Cas6 to the Cas6 recognition sequence reduces translation of the gene of interest. In an example, one or more of the one or more first miR is expressed in the target cell. In still another example, the recombinant second RNA molecule further comprises (iii) a second miR recognition element in its 3′ UTR, wherein the second miR recognition element recognizes one or more second miR and binding of one or more of the one or more second miR to the second miR recognition element reduces translation of the gene of interest. In yet another example, one or more of the one or more second miR is expressed in an off-target cell.

In another example, one or both of the first RNA molecule and the second RNA molecule comprises one or more of a modified ribonucleotide and an anti-reverse cap analog. In still another example, one or both of the first RNA molecule and the second RNA molecule comprises one or more modified ribonucleotide and the one or more modified ribonucleotide is independently selected from pseudouridine and cytidine. In yet another example, one or both of the first RNA molecule and the second RNA molecule comprises an anti-reverse cap analog and the anti-reverse cap analog is selected from 3′-O-Me-m7G(5′)ppp(5′)G cap. In a further example, the target cell is a heart tissue cell, a lung tissue cell, a liver tissue cell, a spleen tissue cell, or a tumor cell. In still a further example, the off-target cell is selected from one or more of a heart tissue cell, a lung tissue cell, a liver tissue cell, a spleen tissue cell, or a tumor cell.

In another example, the target cell is a tumor cell. In another example, the target cell is a breast tumor cell. In yet another example, the off-target cell is selected from one or more of a heart tissue cell, a lung tissue cell, a liver tissue cell, a spleen tissue cell, or a tumor cell. In a further example, the gene of interest encodes a protein and the protein is selected from an anti-apoptotic protein, a pro-apoptotic protein, a cell cycle-inducer protein, and a cell-cycle arrest protein. In still a further example, the gene of interest encodes a pro-apoptotic protein. In yet a further example, the gene of interest encodes an anti-apoptotic protein.

In another example, the gene of interest encodes a cell cycle-inducer protein and the cell cycle-inducer protein is selected from Lin28, Pkm2 and Cyclin D2. In still another example, the gene of interest encodes a cell cycle-arrest protein. In yet another example, the gene of interest encodes an acid ceramidase a type 2 phosphatidylinositol-5-phosphate 4-kinase gamma a Lin28, a Pkm2, a Cyclin D2, a p53 protein, a Herpes Simplex Virus type 1 thymidine kinase, a deltex protein, an E1A protein, a cystatin SA protein, a cystatin E/M protein, or a caspase 9 protein. In a further example, the gene of interest encodes a protein and the protein is selected from an antibody, an anti-angiogenic protein, and an angiogenic protein. In still a further example, the gene of interest encodes a marker protein. In yet a further example, the gene of interest encodes a marker protein and the marker protein is selected from a green fluorescence protein, inactive human CD25, inactive mouse CD25, beta-galactosidase, and luciferase.

In another example, the gene of interest encodes a fluorescent protein. In still another example, the gene of interest encodes a fluorescent protein and the fluorescent protein is selected from a green fluorescent protein, a yellow fluorescent protein, mCherry, and tdTomato. In still another example, the expression regulatory system further comprises a nanoparticle, wherein the nanoparticle comprises the first RNA molecule and the second RNA molecule. In yet another example, the nanoparticle comprises any one or more of a liposome nanoparticle, a gold nanoparticle, an iron nanoparticle, a poly lactic-co-glycolic acid nanoparticle, and a viral vector. In a further example, the nanoparticle comprises a positive charge, a negative charge, or a neutral charge. In still a further example, the second miR recognition element includes one or more of a miR-195a recognition element, a miR-200c recognition element, a miR-Let7f recognition element, a miR-143 recognition element, a miR-222 recognition element, a miR-142a recognition element, a miR-122 recognition element, a miR-146a recognition element, a miR-34c recognition element, a miR-17 recognition element, a miR-125 recognition element, a miR-26a2 recognition element, a miR-92a recognition element, a miR-20a recognition element, and a miR-486a recognition element, a miR-146a recognition element, and any combination of two or more of the foregoing.

In another example, the first miR recognition element comprises one or both of a miR-1 recognition element and a miR-208 recognition element and the second miR recognition element comprises one or both of a miR-143 recognition element and a miR-146a recognition element. In yet another example, the first miR recognition element comprises one or both of a miR-1 recognition element and a miR-208 recognition element and the second miR recognition element comprises one or both of a miR-143 recognition element and a miR-146a recognition element.

In another example, the target cell is a heart tissue cell. In still another example, the one or more off-target cell is selected from one or more of a heart tissue cell, a lung tissue cell, a liver tissue cell, and a spleen tissue cell. In yet another example, the target cell is a cardiomyocyte. In a further example, one or more of the one or more off-target cells is a non-cardiomyocyte heart tissue cell.

In another example, the expression regulatory system further comprises a nanoparticle, wherein the nanoparticle comprises the first RNA molecule, the second RNA molecule, and a positive charge. In still another example, the gene of interest encodes an acid ceramidase a type 2 phosphatidylinositol-5-phosphate 4-kinase gamma a Lin28, a Pkm2, or a Cyclin D2. In yet another example, the first miR recognition element comprises one or more of a miR-155 recognition element, a miR10b recognition element, a miR181a recognition element, and miR-181b recognition element. In a further example, the second miR recognition element comprises one or more of a miR143 recognition element and a miR122 recognition element. In still a further example, the first miR recognition element comprises one or more of a miR-155 recognition element, a miR10b recognition element, a miR181a recognition element, and miR-181b recognition element, and the second miR recognition element comprises one or more of a miR143 recognition element and a miR122 recognition element.

In another example, the first miR recognition element comprises a miR-155 recognition element. In still another example, the second miR recognition element comprises a miR122 recognition element. In yet another example, the first miR recognition element comprises a miR-155 recognition element and the second miR recognition element comprises a miR122 recognition element. In a further example, the target cell comprises a tumor cell. In still a further example, the tumor cell comprises a breast tumor cell. In still a further example, the off-target cell is selected from one or more of a heart tissue cell, a lung tissue cell, a liver tissue cell, and a spleen tissue cell. In yet a further example, a nanoparticle, wherein the nanoparticle comprises the first RNA molecule, the second RNA molecule, and a neutral charge.

In another example, the gene of interest encodes a p53 protein, a Herpes Simplex Virus type 1 thymidine kinase, a deltex protein, an E1A protein, a cystatin SA protein, a cystatin E/M protein, or a caspase 9 protein. In still another example, the gene of interest encodes a tumor-suppressor protein. In yet another example, the first miR recognition element comprises a miR-146a recognition element. In a further example, the second miR recognition element comprises a miR143 recognition element. In still a further example, the first miR recognition element comprises a miR-146a recognition element and the second miR recognition element comprises a miR143 recognition element. In yet a further example, the target cell comprises a lung tissue cell. In another further example, the lung tissue cell comprises a myofibroblast.

In another example, the off-target cell is selected from one or more of a heart tissue cell, a liver tissue cell, and a spleen tissue cell. In an example, the off-target cell comprises a lung tissue cell, and the lung tissue cell is not a myofibroblast. In still another example, the expression regulatory system further comprises a nanoparticle, wherein the nanoparticle comprises the first RNA molecule, the second RNA molecule, and a positive charge. In yet another example, the gene of interest encodes a type 2 phosphatidylinositol-5-phosphate 4-kinase gamma. In a further example, the first miR recognition element comprises one or more of a miR-146a recognition element, a miR-20 recognition element, a miR-148 recognition element, and a miR-223 recognition element. In still a further example, the first miR recognition element comprises one or more of a miR-20 recognition element, a miR-148 recognition element, and a miR-223 recognition element.

In another example, the first miR recognition element comprises a miR-146a recognition element. In still another example, the first miR recognition element comprises a miR-20 recognition element. In yet another example, the first miR recognition element comprises a miR-148 recognition element. In a further example, the first miR recognition element comprises a miR-223 recognition element. In still a further example, the target cell comprises a monocyte. In yet a further example, the off-target cell comprises a bone marrow cell wherein the bone marrow cell is not a monocyte. In another example, the nanoparticle comprises the first RNA molecule, the second RNA molecule, and a negative charge.

In a further aspect, provided is a method, comprising administering the expression regulatory system to a subject, wherein the subject suffered a myocardial infarction or suffers from heart failure. In another example, the expression regulatory system further comprises a nanoparticle and the nanoparticle comprises the first RNA molecule, the second RNA molecule, and a positive charge. In still another example, administering comprises administering the nanoparticle intravenously. In yet another example, the administering comprises administering by intramyocardial injection. In a further example, the administering comprises administering two or more times. In still a further example, the administering comprises stimulating proliferation of cardiomyocytes.

In another example, the second miR recognition element includes one or more of a miR-195a recognition element, a miR-200c recognition element, a miR-Let7f recognition element, a miR-143 recognition element, a miR-222 recognition element, a miR-142a recognition element, a miR-122 recognition element, a miR-146a recognition element, a miR-34c recognition element, a miR-17 recognition element, a miR-125 recognition element, a miR-26a2 recognition element, a miR-92a recognition element, a miR-20a recognition element, and a miR-486a recognition element, a miR-146a recognition element, and any combination of two or more of the foregoing. In still another example, the first miR recognition element comprises one or both of a miR-1 recognition element and a miR-208 recognition element. In yet another example, the second miR recognition element comprises one or both of a miR-143 recognition element and a miR-146a recognition element.

In another example, the first miR recognition element comprises one or both of a miR-1 recognition element and a miR-208 recognition element and the second miR recognition element comprises one or both of a miR-143 recognition element and a miR-146a recognition element. In still another example, the target cell is a heart tissue cell. In still another example, the off-target cell is selected from one or more of a heart tissue cell, a lung tissue cell, a liver tissue cell, and a spleen tissue cell. In yet another example, the target cell is a cardiomyocyte. In yet another example, the off-target cells is a non-cardiomyocyte heart tissue cell. In a further example, the expression regulatory system further comprises a nanoparticle, wherein the nanoparticle comprises the first RNA molecule, the second RNA molecule, and a positive charge. In still a further example, the gene of interest encodes an acid ceramidase, a type 2 phosphatidylinositol-5-phosphate 4-kinase gamma a Lin28, a Pkm2, or a Cyclin D2.

In still a further aspect, provided is a method, comprising administering the expression regulatory system to a subject, wherein the subject suffers from cancer. example, the subject suffers from cancer. In an example, the subject suffers from breast cancer. In another example, the expression regulatory system further comprises a nanoparticle and the nanoparticle comprises the first RNA molecule, the second RNA molecule, and a neutral charge. In still another example, administering comprises administering the nanoparticle intravenously. In yet another example, the administering comprises administering by intratumoral injection. In a further example, the administering comprises administering two or more times. In still a further example, the administering comprises inhibiting tumor growth.

In another example, the second miR recognition element includes one or more of a miR-195a recognition element, a miR-200c recognition element, a miR-Let7f recognition element, a miR-143 recognition element, a miR-222 recognition element, a miR-142a recognition element, a miR-122 recognition element, a miR-146a recognition element, a miR-34c recognition element, a miR-17 recognition element, a miR-125 recognition element, a miR-26a2 recognition element, a miR-92a recognition element, a miR-20a recognition element, and a miR-486a recognition element, a miR-146a recognition element, and any combination of two or more of the foregoing. In still another example, the first miR recognition element comprises one or more of a miR-155 recognition element, a miR10b recognition element, a miR18-la recognition element, and miR-181b recognition element. In yet another example, the second miR recognition element comprises one or more of a miR143 recognition element and a miR122 recognition element. In a further example, the first miR recognition element comprises one or more of a miR-155 recognition element, a miR10b recognition element, a miR181a recognition element, and miR-181b recognition element, and the second miR recognition element comprises one or more of a miR143 recognition element and a miR122 recognition element. In still a further example, the first miR recognition element comprises a miR-155 recognition element. In yet a further example, the second miR recognition element comprises a miR122 recognition element.

In another example, the first miR recognition element comprises a miR-155 recognition element and the second miR recognition element comprises a miR122 recognition element. In still another example, the target cell comprises a tumor cell. In yet another example, the tumor cell comprises a breast tumor cell. In a further example, the off-target cell is selected from one or more of a heart tissue cell, a lung tissue cell, a liver tissue cell, and a spleen tissue cell. In still a further example, the gene of interest encodes a p53 protein, a Herpes Simplex Virus type 1 thymidine kinase, a deltex protein, an E1A protein, a cystatin SA protein, a cystatin E/M protein, or a caspase 9 protein. In another further example, the gene of interest encodes a tumor-suppressor protein.

In yet a further aspect, provided is a method comprising administering the expression regulatory system to a subject, wherein the subject suffers pulmonary fibrosis. In an example, the expression regulatory system further comprises a nanoparticle and the nanoparticle comprises the first RNA molecule, the second RNA molecule, and a neutral charge. In another example, administering comprises administering the nanoparticle intravenously. In still another example, the administering comprises administering by intrapulmonary injection. In yet another example, the administering comprises administering two or more times. In a further example, the administering comprises reducing pulmonary fibrosis.

In another example, the second miR recognition element includes one or more of a miR-195a recognition element, a miR-200c recognition element, a miR-Let7f recognition element, a miR-143 recognition element, a miR-222 recognition element, a miR-142a recognition element, a miR-122 recognition element, a miR-146a recognition element, a miR-34c recognition element, a miR-17 recognition element, a miR-125 recognition element, a miR-26a2 recognition element, a miR-92a recognition element, a miR-20a recognition element, and a miR-486a recognition element, a miR-146a recognition element, and any combination of two or more of the foregoing. In still another example, the first miR recognition element comprises a miR-146a recognition element. In yet another example, the second miR recognition element comprises a miR143 recognition element.

In another example, the target cell comprises a lung tissue cell. In still another example, the off-target cell is selected from one or more of a heart tissue cell, a liver tissue cell, and a spleen tissue cell. In yet another example, the off-target cell is a lung tissue cell, and the lung tissue cell is not a myofibroblast. In a further example, the gene of interest encodes a type 2 phosphatidylinositol-5-phosphate 4-kinase gamma.

In another further aspect, provided is a method, comprising administering the expression regulatory system to a subject. In another example, the expression regulatory system further comprises a nanoparticle, and a nanoparticle comprises the first RNA molecule and the second RNA molecule. In still another example, the administering comprises intravenous administration.

### BRIEF DESCRIPTION OF THE DRAWINGS

These and other features, aspects, and advantages of the present disclosure will become better understood when the following detailed description is read with reference to the accompanying drawings, wherein:

FIGS. 1A and 1B show non-limiting examples of expression regulatory systems in accordance with aspects of the present disclosure.

FIGS. 2A-2M show CM-SMRTs 2.0 structure, organ and cell specificity and pharmacokinetics post minimal invasive delivery.

FIGS. 3A-3K show attenuation of cell death and inflammatory response post minimal invasive delivery of a non-limiting example of an acid ceramidase (AC) CM-SMRT 2.0 in an Ischaemia-Reperfusion (I/R) cardiac injury model.

FIGS. 4A-4H show cardiac protection and decrease cardiac remodeling post minimal invasive delivery of a non-limiting example of an AC CM-SMRT 2.0 in an I/R injury model.

FIG. 5A-5F show non-limiting examples of miR recognition elements that may reduce modRNA translation in different cells of different tissues post I.V injection.

FIGS. 6A-6I show a comparison of I.V delivery of a non-limiting examples of a Luc CM SMRT or of a CM SMRT 2.0 with or without cardiac IR injury.

FIGS. 7A-7H shows a demonstration of effects of intramyocardial injection with an example of Luc or of AC modRNA or with an example of AC SMRT 2.0 in preventing cardiac remodeling post I/R injury.

FIGS. 8A-8F show expression in vitro and in vivo of on 4T1 breast tumor cells of a gene of interest in an example of SMRT in accordance with aspects of the current disclosure.

FIGS. 9A-9E show inhibition of tumor growth by non-limiting examples of SMRT carrying different genes of interest in accordance with aspects of the present disclosure.

FIGS. 10A-10E illustrate delivery of an example of breast tumor expression of a gene of interest in non-limiting examples of SMRT 2.0 in accordance with aspects of the present disclosure.

FIGS. 11A-11F show intravenous delivery of an example of SMRT modRNA (Cas6 with miR-146a recognition element and type 2 phosphatidylinositol-5-phosphate 4-kinase gamma (Pip4k2c) or Luc as gene of interest), and expression in cells of lung tissue.

FIGS. 12A-12C show a non-limiting examples of SMRT in accordance with aspects of the present disclosure with Pip4k2c or mCherry as gene of interest and Cas6 with miR-146a recognition element modRNA, or dnTGFb modRNA, reducing fibrosis in the lung.

FIGS. 13A-13C show minimally invasive SMRTs delivery to monocytes. in vitro expression of a gene of interest in monocytes (CD11b+) following in vitro treatment of adhered mouse bone marrow cells with a non-limiting example SMRT (Cas6 modRNA with miR146a, miR20, miR148, or miR223 response element, and mCherry modRNA with a Cas6-recognition hairpin). Adhered mouse bone marrow cells were transfected or not with nuclear mCherry with hairpin modRNA or with nuclear mCherry monocytes SMRT based on different miR recognition sites (miR146a, miR20, miR148, miR223). 24h later cells were fixed and stained for monocytes marker CD11b (green). B. Quantification of nuclear mCherry expression in monocytes (CD11b+) vs non-monocytes (CD11b−) cells. miR20, miR148, miR223, yielded exclusive expression in monocytes (CD11b+). C. Bioluminescent image of Hek cells (cell line derived from human embryonic kidney cells, lacking monocytes) as a non-target cell, plated in 12 well plate transfected or not with Luc with hairpin modRNA or SMRT based (Cas6 modRNA with miR146a, miR20, miR148, miR223 recognition element, and Luc with Cas6 hairpin response element). Luc modRNA translate well in Hek cells but these examples of SMRT do not.

FIGS. 14A-14J show the safety prolife of different modRNA delivered with positively charged nanoparticles.

FIG. 15 shows quantification of bioluminescent IVIS images if HEK cells transfected with modRNA encoding CAS6 carrying miR1-208 and Luc modRNA with Cas6 hairpin (n=3). Unpaired t-test was used.

FIG. 16A-16M show evaluation of CM SMRTs based on Cas6 expression in the heart.

FIGS. 17A-17C show minimal invasive delivery of Pip4k2c breast tumor SMRT 2.0 and anti-CTLA-4 antibody modRNA significantly reduce tumor volume and weight.

FIGS. 18A-18G show a comparison of intravenous vs intratracheal delivery of Lung SMRTs.

### DETAILED DESCRIPTION

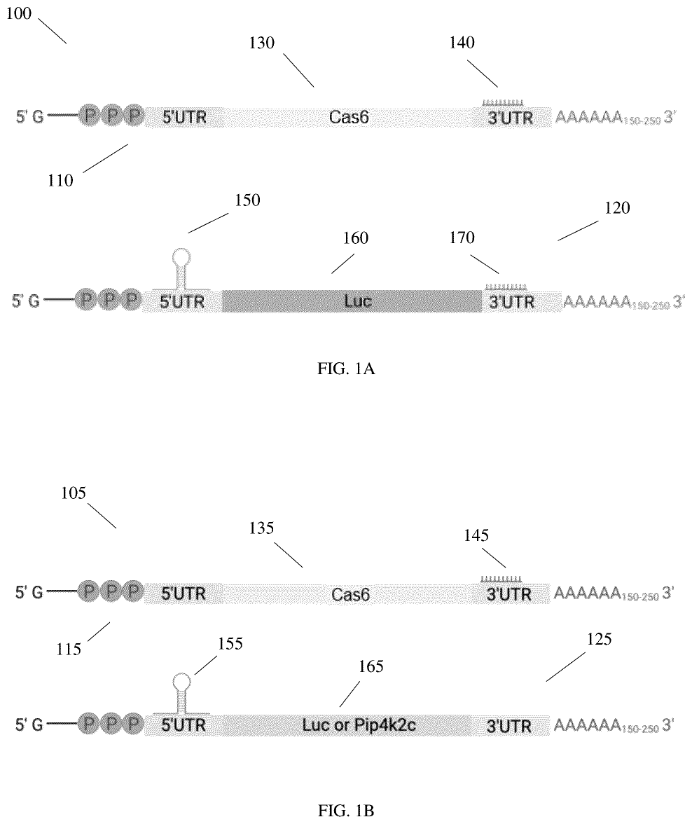

This disclosure relates to an expression regulatory system for expression of a gene of interest in a target cell. Representative, non-limiting examples of such a system are illustrated in FIGS. 1A and 1B. In a non-limiting, representative example in FIG. 1A, an example of an expression regulatory system 100 is shown. A system may include two recombinant RNA molecules. A first RNA molecule 110 may include a coding sequence of a translation suppressor protein 130. In this example, a translation suppressor protein Cas6 (also known as CSY4 and referred to interchangeable herein as Cas6 or CSY4 without distinction) is shown, though a coding sequence for another translation suppressor protein may alternatively be included in a different example in accordance with aspects of the present disclosure. The first RNA molecule may also include a first microRNA (miR) recognition element 140 in its 3′ UTR, wherein the first miR recognition element recognizes one or more first miR and binding of one or more of the one or more first miR to the first miR recognition element reduces translation of the translation suppressor. Thus, uptake of the first RNA molecule 110 by a cell may result in expression of the translation suppressor protein therein, following translation by the translation machinery and, in an example any pertinent post-translational processing machinery, of the cell, including uptake by a non-target cell. In a target cell, however, expression of a miR, which recognizes the first miR recognition element 140, may reduce translation of the translation suppressor protein from the first RNA molecule. Thus, in an example, low, little, undetectable, or no expression the translation suppressor protein may result in a target cell.

Continuing with the example of FIG. 1A, an expression regulatory system as disclosed herein may further include a second RNA molecule 120. The second RNA molecule 120 may include a coding sequence for a gene of interest 160. In the non-limiting example illustrated in FIG. 1A, luciferase (Luc) is illustrated as a gene of interest 160, though any other gene of interest may be included, where expression thereof may be desirable in a target cell. The second RNA molecule may also include a recognition sequence for the translation-suppressor 150, wherein binding of the translation-suppressor to the recognition sequence for the translation-suppressor 150 reduces translation of the gene of interest. In the non-limiting example illustrated in FIG. 1A, the recognition sequence for the translation-suppressor 150 is represented by a hairpin RNA structure 5′ to the coding sequence of the gene of interest 160. Cas6 is known to recognize and bind to an RNA hairpin-forming recognition sequence and cleave the RNA molecule to which it binds. In this example, the recognition sequence for the translation-suppressor 150 is positioned such that recognition and cleavage by the Cas6 translation suppressor inhibits translation of the gene of interest encoded by the second RNA molecule 120.

Thus, uptake of the second RNA molecule 120 by a non-target cell that also uptakes that first RNA molecule 110 may result in little, low, no, or undetectable expression of the protein product of the gene of interest, because expression of (for example) Cas6 (or, in another example, another translation suppressor protein wherein the second RNA molecule includes a corresponding recognition sequence therefor) as translated from the first RNA molecule 110 would inhibit, suppress, reduce, or eliminate translation of the gene of interest 160. However, in a target cell that takes up the second RNA molecule 120 in addition to the first RNA molecule 110, presence of the miR recognition sequence in the first RNA molecule and binding thereto by a corresponding miR expressed in a target cell results in reduced, low, blunted, no, or undetectable translation suppressor (e.g., Cas6) expression in a target cell. The translation suppressor would therefore not bind to its recognition sequence 150 on a second RNA molecule 120 in a target cell nor cleave it or reduce translation of the gene of interest encoded for by the second RNA molecule 160. Thus, compared to a non-target cell having uptake of the first RNA molecule 110 and second RNA molecule 120, wherein uninhibited expression of the translation suppressor would negatively impact expression of the gene of interest, a target cell having uptake of the first RNA molecule 110 and second RNA molecule 120 would have higher expression of the gene of interest.

In some examples, however, translation suppressor expression in a non-target cell might not reduce expression levels of a gene of interest to as low a level as may be preferred. Or in any event tighter limits on expression of a gene of interest in a non-target cell may be desirable. Thus, in the example illustrated in FIG. 1A, the second RNA molecule may further include a second miR recognition element in its 3′ UTR 170, wherein the second miR recognition element recognizes one or more second miR and binding of one or more of the one or more second miR to the second miR recognition element reduces translation of the gene of interest. For example, one or more miR may be expressed in one or more non-target cell, and binding thereof to the second miR recognition element 170 may reduce, inhibit, minimize, or eliminate expression of the gene of interest from the second RNA molecule 120. Such diminution of expression of the gene of interest from the second RNA molecule by binding of the one or more second miR to the recognition sequence therefore in the second RNA molecule 120 in an off-target cell, in addition to inhibitory effects of translation suppressor therein. These two effects, of translation suppressor expression and second miR expression in a non-target cell, may, individually, additively, or synergistically, inhibit translation of the gene of interest from the second RNA molecule in a non-target cell from the second RNA molecule 120, as disclosed herein.

In another non-limiting, representative example in FIG. 1B, an example of an expression regulatory system 105 is shown. A system may include two recombinant RNA molecules. A first RNA molecule 115 may include a coding sequence of a translation suppressor protein 135. In this example, the translation suppressor protein is Cas6 The first RNA molecule may also include a first miR recognition element 145 in its 3′ UTR, wherein the first miR recognition element 145 recognizes one or more first miR and binding of one or more of the one or more first miR to the first miR recognition element 145 reduces translation of the translation suppressor Cas6. Thus, uptake of the first RNA molecule 115 by a cell may result in expression of the translation suppressor protein Cas6 therein, following translation by the translation machinery and, in an example any pertinent post-translational processing machinery, of the cell, including uptake by a non-target cell. In a target cell, however, expression of a miR, which recognizes the first miR recognition element 145, may reduce translation of the translation suppressor Cas6 135 of the first RNA molecule. Thus, in an example, low, little, undetectable, or no expression the translation suppressor protein may result in a target cell.

Continuing with the example of FIG. 1B, an expression regulatory system as disclosed herein may further include a second RNA molecule 125. The second RNA molecule 125 may include a coding sequence for a gene of interest 165. In the non-limiting example illustrated in FIG. 1B, luciferase (Luc) or type 2 phosphatidylinositol-5-phosphate 4-kinase gamma (Pip4k2c) are illustrated as examples of a gene of interest 165, though any other gene of interest may be included, where expression thereof may be desirable in a target cell. The second RNA molecule may also include a recognition sequence for the translation-suppressor Cas6 155, wherein binding of the translation-suppressor to the recognition sequence for the translation-suppressor 155 reduces translation of the gene of interest. In the non-limiting example illustrated in FIG. 1B, the recognition sequence for the translation-suppressor 155 is represented by a hairpin RNA structure 5′ to the coding sequence of the gene of interest 165. In this example, the recognition sequence for the translation-suppressor 155 is positioned such that recognition and cleavage by the Cas6 translation suppressor inhibits translation of the gene of interest encoded by the second RNA molecule 125.

Thus, uptake of the second RNA molecule 125 by a non-target cell that also uptakes that first RNA molecule 115 may result in little, low, no, or undetectable expression of the protein product of the gene of interest, because expression of the, in this example, translation suppressor Cas6 as translated from the first RNA molecule 115 would inhibit, suppress, reduce, or eliminate translation of the gene of interest 165. However, in a target cell that takes up the second RNA molecule 125 in addition to the first RNA molecule 115, presence of the miR recognition sequence in the first RNA molecule and binding thereto by a corresponding miR expressed in a target cell results in reduced, low, blunted, no, or undetectable Cas6 expression protein and expression in a target cell. Cas6 would therefore not bind to its recognition sequence 150 on a second RNA molecule 120 in a target cell nor cleave it or reduce translation of the gene of interest encoded for by the second RNA molecule 160. Thus, compared to a non-target cell having uptake of the first RNA molecule 110 and second RNA molecule 120, wherein uninhibited expression of the translation suppressor would negatively impact expression of the gene of interest, a target cell having uptake of the first RNA molecule 110 and second RNA molecule 120 would have higher expression of the gene of interest.

In some non-limiting examples, however, Cas6 expression in a non-target cell might not reduce expression levels of a gene of interest to as low a level as may be preferred. Or in any event tighter limits on expression of a gene of interest in a non-target cell may be desirable. Thus, the second RNA molecule shown in FIG. 1B may further include a second miR recognition element in its 3′ UTR (not shown), wherein the second miR recognition element recognizes one or more second miR and binding of one or more of the one or more second miR to the second miR recognition element reduces translation of the gene of interest. For example, one or more miR may be expressed in one or more non-target cell, and binding thereof to the second miR recognition element (not shown) may reduce, inhibit, minimize, or eliminate expression of the gene of interest from the second RNA molecule 125. Such diminution of expression of the gene of interest from the second RNA molecule by binding of the one or more second miR to the recognition sequence therefore in the second RNA molecule 125 in an off-target cell, in addition to inhibitory effects of Cas6 therein. These two effects, of Cas6 expression and second miR expression in a non-target cell, may individually, additively, or synergistically inhibit translation of the gene of interest from the second RNA molecule in a non-target cell from the second RNA molecule 120, as disclosed herein.

Translation Suppressor

A translation suppressor may be a factor that binds to or associates with a recognition sequence in an RNA molecule and inhibits, prevents, reduces, eliminates, or otherwise diminishes translation of a protein encoded by the RNA molecule. An example disclosed herein include Cas6 (a.k.a. Csy4). Cas6 is a component of CRISPRs (Clustered Regularly Interspaced Short Palindromic Repeats) found in prokaryotes as protection against viruses and other foreign polynucleotides. Cas6 is an endoribonuclease that recognizes and binds a hairpin formation in substrate RNA formed by a recognition sequence. A substrate RNA molecule of Cas6 is cleaved by Cas6, e.g. at or near the 3′ end of the hairpin stem. For an RNA molecule that includes the Cas6 recognition sequence, such as in the 5′ UTR of the RNA molecule or after the start codon of a coding sequence for a gene of interest included therein, co-expression with Cas6 may result in degradation of the RNA molecule and reduction in expression of the gene of interest. In an example disclosed herein, a first RNA molecule encodes a translation suppressor such as Cas6, and a second RNA molecule encodes a gene of interest and includes a recognition sequence for the translation suppressor such as Cas6. In an example, the recognition sequence may be in the 5′ UTR of the gene or interest, within one or more nucleotides 5′ to the start codon, or one or more nucleotides 3′ to the start codon, or anywhere in between. In another example, the recognition sequence may be present anywhere in a second RNA molecule wherein binding to or recognition thereof by a translation suppressor such as Cas6 leads to a reduction in translation of the gene of interest encoded by the second RNA molecule.

A translation suppressor other than Cas6 and associated recognition element therefor may also be included in a second RNA molecule of an expression regulatory system as disclosed herein. Another, non-limiting example includes the archaeal translation suppression protein is L7Ae, or a eukaryotic homolog thereof such as L30e. Such factors are RNA binding proteins that repress translation of a gene of interest encoded for by a second RNA molecule. The recognition sequence therefor known as a kink-turn, k-motif, or k-turn. L7Ae and L30e binding to such recognition sequence inhibits the expression of a gene of interest encoded by a second RNA molecule. For an RNA molecule that includes kink-turn, k-motif, or k-turn, co-expression with, for example, L7Ae or L30e may result in suppression of translation and reduction in expression of the gene of interest encoded thereby. In an example disclosed herein, a first RNA molecule encodes a translation suppressor such as L7Ae or L30e, and a second RNA molecule encodes a gene of interest and includes a kink-turn, k-motif, or k-turn recognition sequence for the translation suppressor such as L7Ae or L30e. In an example, the recognition sequence may be in the 5′ UTR of the gene of interest, within one or more nucleotides 5′ to the start codon, or one or more nucleotides 3′ to the start codon, or anywhere in between. The recognition sequence may be present anywhere in a second RNA molecule wherein binding to or recognition thereof by a translation suppressor such as L7Ae or L30e leads to a reduction in translation of the gene of interest encoded by the second RNA molecule.

Other examples of an expression regulatory system as disclosed herein may include, in a first RNA molecule, a coding sequence for a translation suppressor other than Cas6, LA7e, or L30e. Accordingly, other examples of an expression regulatory system as disclosed herein may also include, in a second RNA molecule, a recognition sequence for said other translation suppressor, operatively associated with a coding sequence for a gene of interest such that interaction of the translation suppressor negatively regulates, suppresses, inhibits, diminishes, eliminates, or otherwise reduces translation of the gene of interest.

Nucleotide sequences encoding for non-limiting examples of translation suppressors and nucleotide sequences for non-limiting examples of recognition sequences for translation suppressors are provided in Table 1 and Table 2, respectively:

TABLE 1
Nucleotide sequences encoding for non-limiting
examples of translation suppressors

| SEQ | Translation | |
|---|---|---|
| ID NO | suppressor | Nucleotide sequence |
| 1 | Cas6 | AUGGACCACUACCUCGACAUUCGCUUGCGACC |
| | | GGACCCGGAAUUUCCCCCGGCGCAACUCAUGA |
| | | GCGUGCUCUUCGGCAAGCUCCACCAGGCCCUG |
| | | GUGGCACAGGGCGGGGACAGGAUCGGCGUGAG |
| | | CUUCCCCGACCUCGACGAAAGCCGCUCCCGGC |
| | | UGGGCGAGCGCCUGCGCAUUCAUGCCUCGGCG |
| | | GACGACCUUCGUGCCCUGCUCGCCCGGCCCUG |
| | | GCUGGAAGGGUUGCGGGACCAUCUGCAAUUCG |
| | | GAGAACCGGCAGUCGUGCCUCACCCCACACCG |
| | | UACCGUCAGGUCAGUCGGGUUCAGGCGAAAAG |
| | | CAAUCCGGAACGCCUGCGGCGGCGGCUCAUGC |
| | | GCCGGCACGAUCUGAGUGAGGAGGAGGCUCGG |
| | | AAACGCAUUCCCGAUACGGUCGCGAGAGCCUU |
| | | GGACCUGCCCUUCGUCACGCUACGCAGCCAGA |
| | | GCACCGGACAGCACUUCCGUCUCUUCAUCCGC |
| | | CACGGGCCGUUGCAGGUGACGGCAGAGGAAGG |
| | | AGGAUUCACCUGUUACGGGUUGAGCAAAGGAG |
| | | GUUUCGUUCCCUGGUUCUGA |
| 2 | L7Ae | AUGUACGUGAGAUUUGAGGUUCCUGAGGACAU |
| | | GCAGAACGAAGCUCUGAGUCUGCUGGAGAAGG |
| | | UUAGGGAGAGCGGUAAGGUAAAGAAAGGUACC |
| | | AACGAGACGACAAAGGCUGUGGAGAGGGGACU |
| | | GGCAAAGCUCGUUUACAUCGCAGAGGAUGUUG |
| | | ACCCGCCUGAGAUCGUUGCUCAUCUGCCCCUC |
| | | CUCUGCGAGGAGAAGAAUGUGCCGUACAUUUA |
| | | CGUUAAAAGCAAGAACGACCUUGGAAGGGCUG |
| | | UGGGCAUUGAGGUGCCAUGCGCUUCGGCAGCG |
| | | AUAAUCAACGAGGGAGAGCUGAGAAAGGAGCU |
| | | UGGAAGCCUUGUGGAGAAGAUUAAAGGCCUUC |
| | | AGAAGUAA |
| 3 | L30e | AUGUCAGGUGAUGAGGCUGCCCCUGUUGUUGU |
| | | UCCUCCCCCCGUUGCUGAGCCAGCGGCCAUCC |
| | | CAGAGGACAUGGACUUAAUGACUGCAUUGGAG |
| | | UUGACUCUUAGGAAAGCUCGUGCUUAUGGUGG |
| | | UGUUGUUCGUGGUCUCCAUGAGUGUGCUAAGC |
| | | UUAUUGAGAAGCGUGUGGCUCAACUCGUUGUC |
| | | UUGGCUGAAGACUGCAACCAGCCUGAUUAUGU |
| | | CAAGCUUGUGAAGGCUCUCUGUGCUGAUCACG |
| | | AAGUCAGGUUGCUAACAGUUCCAAGUGCCAAG |
| | | ACCCUUGGCGAAUGGGCUGGUCUCUGCAAGAU |
| | | UGAUUCUGAGGGUAAUGCCAGGAAGGUUGUUG |
| | | GAUGCUCAUGUCUUGUUGUCAAGGACUUCGGC |
| | | GAGGAGACAACUGCCCUCAGUAUCGUCAAUAA |
| | | GCAUAUUGCUUCUCAAUAA |

TABLE 2
Nucleotide sequences of non-limiting
examples of recognition
elements for translation suppressors

| SEQ | Translation | Nucleotide sequence for |
|---|---|---|
| ID NO | suppressor | recognition element |
| 4 | Cas6 | GUUCACUGCCGUAUAGGCAGCUAAGAAA |
| 5 | L7Ae | AAAGGUGGGCGUGAUCCGAAAGGUGACCC |
| | | GGAUCUGGGGCGUGAUCCGAAAGGUGACC |
| | | CGGAA |
| 6 | L30e | UACCCCCGGAGUGUUUUAUACGCAGAGAU |
| | | GGAGACAACAAG |

A recombinant RNA molecule as disclosed herein may also include a nucleotide sequence that encodes for a translation suppressor such as set out in Table 1, though the nucleotide sequence therefor may differ from the corresponding sequence as set out in Table 1 owing to, for example, codon redundancy. As skilled persons would also appreciate, an amino acid sequence of a translation suppressor may vary from a sequence encoded by a nucleotide sequence of Table 1, such as an isoform of, for example, Cas6, L7Ae, or L30e, while still functioning as a translation suppressor, and a recombinant RNA molecule as disclosed herein may encode such isoform or variant, such as when another recombinant RNA molecule in accordance with the present disclosure includes a corresponding translation suppressor nucleotide sequence recognition element (e.g. as set out in Table 2, or an equivalent thereof). For example, an amino acid sequence of a translation suppressor protein encoded by a recombinant RNA molecule in accordance with the present disclosure may be less than 10000 homologous to an amino acid sequence of a translation suppressor protein encoded by a nucleotide sequence of Table 1. For example, an amino acid sequence of a translation suppressor protein encoded by a recombinant RNA molecule in accordance with the present disclosure may be 99% or more, or 97% or more, or 95% or more, or 92% or more, or 90% or more, or 87% or more, or 85% or more, or 80% or more, or 75% or more, or 75% or more homologous to an amino acid sequence of a translation suppressor protein encoded by a nucleotide sequence of Table 1

microRNA (miR) and miR Recognition Element

A miR is a ribonucleic acid sequence having complementarity to a recognition element, which is a portion of a coding RNA strand. Members of the miR family are non-coding RNA polynucleotides, often of from approximately 18-25 nucleotides in length, that regulate gene expression by targeting, e.g., messenger RNA in a sequence specific manner, inducing translational repression or RNA degradation depending on the degree of complementarity between miR and their targets. Upon binding to a complementary recognition sequence or element (which may be complementary to a portion of a miR for which it is a recognition element), which may be but need not be located in the 3′ UTR of an RNA molecule, miR may suppress translation from RNA molecules that include such miR recognition sequence. Cells express endogenous mature miRs, post-transcriptionally regulating mRNAs that have miR recognition sequences with complementarity to the bound miRNA. Through the hybridization of the anti-miRNA sequence to the miRNA sequence, the function of the miRNA sequence is neutralized by preventing its selective binding to the target.

Different cell types may express one or more miR that differ from miR expressed by other cell types. Some different cell types may express some of the same miR as each other and also express miR not expressed by the or another cell type. Different cells within a tissue of a given organ may be distinguishable from other cell types, other cells of a tissue type, or other tissue cells of a given organ, or cells of a different organ, based on whether they do or do not express a species of miR. Different cells within a tissue of a given organ may also be distinguishable from other cell types, other cells of a tissue type, or other tissue cells of a given organ, or cells of a different organ, based on whether transcription of an RNA molecule in said cell, whether endogenous or transfected to the cell, possessing a miR recognition element that recognizes a given species of miR may be reduced, inhibited, blocked, or diminished relative to transcription in other cells, such as resulting from the differential expression of the corresponding miR in the different cell types. In a given cell, an RNA molecule including a coding sequence for a gene of interest and a recognition sequence for a miR expressed in said cell may be translated less than it is in another cell that does not express said miR, for example.

In some examples, an RNA molecule may include a recognition sequence for more than one type of miR. For example, it be desirable to suppress translation from the RNA molecule in, for example, two cell types but not in a third. In a hypothetical example, cell type A expresses miR A, which inhibits translation from an RNA molecule that includes a recognition sequence for miR A, cell type B expresses miR B which inhibits translation from an RNA molecule that includes a recognition sequence for miR B, and cell type C expresses neither miR A nor miR B. An RNA molecule with a coding sequence for a gene of interest, a miR A recognition sequence, and a miR B recognition sequence may be transfected into each cell type. Translation of the gene of interest may be inhibited in cell type A, for example because of interaction of miR A expressed by cell type A with the miR A recognition sequence in the RNA molecule, and translation of the gene of interest may be inhibited in cell type B, for example because of interaction of miR B expressed by cell type B with the miR B recognition sequence in the RNA molecule. In contrast, translation in cell type C may not be similarly inhibited, lacking as cell type C does in expression of miR A and miR B.

In another example, an RNA molecule may include a recognition sequence for each of more than one miR species expressed by a cell type. In a hypothetical example, RNA molecule I may have a miR recognition sequence for miR X, RNA molecule II may have a miR recognition sequence for miR Y, and RNA molecule III may have a miR recognition sequence for miR X and a miR recognition sequence for miR X. Cell type Z may express miR X and miR Y. Transfection of a cell of cell type Z with an RNA molecule including a miR X recognition element but no miR Y recognition element, or with an RNA molecule including a miR Y recognition element but no miR X recognition element, may result in less translation from the RNA molecule than does transfection of a cell of cell type Z with an RNA molecule lacking a recognition sequence for miR X and for miR Y, owing for example to inhibitory effects of the miR X or miR Y, respectively, expressed in such cells on translation from RNA molecules I or II. However, there may still be some translation from RNA molecule I or II in cells of type Z because the miR X or miR Y expressed by cell type Z may reduce but not eliminate translation therefore, or may reduce it by an amount less than may be desired or intended. By comparison, translation from RNA molecule III in cells of type Z may be lower than of transcription of RNA molecule I or II, because, for example, the additive, combinatorial, or synergistic translational inhibitory effects of miR X and miR Y expressed by cells of type Z may inhibit translation more than either miR alone.

In other examples, an RNA molecule may have a miR recognition sequence, for more than one miR, such as for two miR, three miR, four miR, five miR, six miR, seven miR, eight miR, nine miR, ten miR, or more. In another example, an RNA molecule may not have a recognition sequence for a miR.

In an example, a first RNA molecule with a coding sequence for a translation suppressor (e.g., Cas6, LA7e, L30e, etc.) may include one or more first miR recognition sequence, and a target cell may express one or more miR that recognized one or more of the one or more first miR recognition sequence included in the first RNA molecule. Binding of a first miR expressed by the target cell may inhibit, prevent, reduce, or eliminate expression of the translation suppressor relative to expression in an off-target cell, which may not express one or more first miR that is recognized by a the one or more first miR recognition sequences present in the first RNA molecule. When a cell is transfected with such first RNA molecule, and also with a second RNA molecule including a recognition sequence for the translation suppressor and a coding sequence for a gene of interest, levels of expression of the gene of interest may be inversely correlated with the level of expression of the translation suppressor by a target cell and one or more off-target cell. Such level of expression may bear a positive correlation with a level of expression of one or more first miR in the target or off-target cell for which a one or more corresponding recognition sequence is included in the first RNA molecule. That is, interaction with one or more first miR in a target cell may disinhibit expression of the gene of interest from the second RNA molecule in the target cell but not, or relatively less so, in an off-target cell.

In an example, a second RNA molecule, which includes a coding sequence for a gene of interest and a recognition sequence for a translation suppressor, may include one or more second miR recognition sequence, and an off-target cell may express one or more second miR that recognizes one or more of the one or more second miR recognition sequence included in the second RNA molecule. Binding of a second miR expressed by the off-target cell may inhibit, prevent, reduce, or eliminate expression of the gee or interest relative to expression in a target cell, which may not express one or more second miR that is recognized by a the one or more second miR recognition sequence present in the second RNA molecule. When a cell is transfected with such a second RNA molecule encoding a gene of interest, and also with first RNA molecule encoding the translation suppressor for which the second RNA molecule includes a recognition sequence, levels of expression of the gene of interest may be inversely correlated with the level of expression of the one or more second miR expressed by an off-target cell. That is, interaction with one or more second miR in an off-target cell may inhibit expression of the gene of interest from the second RNA molecule in the off-target cell but not, or relatively less so than, in a target cell. In an example, combination of expression of a translation suppressor and one or more second miR in an off target cell may combine to reduce expression of a gene of interest from a second RNA molecule in an off-target cell. In an example, the combined effect equates to a lower level of expression of a gene of interest than results from transfection with a second RNA molecule lacking a miR recognition sequence. In another example, inclusion of exclusion of a second miR recognition sequence from a second RNA molecule may not affect expression of the gene of interest more so that co-transfection with a first RNA molecule including a recognition sequence for a first miR and a coding sequence for a translation suppressor.

In other examples, an RNA molecule may have a miR recognition sequence, for more than one miR, such as for two miR, three miR, four miR, five miR, six miR, seven miR, eight miR, nine miR, ten miR, or more. In another example, an RNA molecule may not have a recognition sequence for a miR. In another example, a second RNA molecule may have no miR recognition sequences. In another example, an RNA molecule may have more than one copy of a recognition sequence for a given miR, such as to increase responsiveness of the RNA molecule to translation-inhibitory effects of the miR.

A first RNA molecule as described herein may include any one or more recognition sequence for any one or more of the following first miR, in any combination: a miR-1 recognition element, a miR-195a recognition element, a miR-200c recognition element, a miR-208 recognition element, a miR-Let7f recognition element, a miR-143 recognition element, a miR-222 recognition element, a miR-142a recognition element, a miR-122 recognition element, a miR-146a recognition element, a miR-34c recognition element, a miR-17 recognition element, a miR-125 recognition element, a miR-26a2 recognition element, a miR-92a recognition element, a miR-20a recognition element, and a miR-486a recognition element.

A second RNA molecule as described herein may include any one or more recognition sequence for any one or more of the following second miR, in any combination: a miR-1 recognition element, a miR-195a recognition element, a miR-200c recognition element, a miR-208 recognition element, a miR-Let7f recognition element, a miR-143 recognition element, a miR-222 recognition element, a miR-142a recognition element, a miR-122 recognition element, a miR-146a recognition element, a miR-34c recognition element, a miR-17 recognition element, a miR-125 recognition element, a miR-26a2 recognition element, a miR-92a recognition element, a miR-20a recognition element, and a miR-486a recognition element.

Nucleotide sequences of non-limiting examples of miR recognition sequences are provided in Table 3, including those used in examples disclosed herein. When expressed by a cell, a microRNA molecule hairpin includes a 3′ end and a 5′ end. In some cases, a cellular microRNA molecule's 3′ end may be active in regulating gene expression by hybridizing to a recognition sequence in an mRNA molecule while in other examples a cellular microRNA molecule's 5′ end may be active in regulating gene expression by hybridizing to a recognition sequence in an mRNA molecule. Table 3 shows non-limiting examples of miR recognition sequences that may be included in recombinant RNA molecules in accordance with the present disclosure and that may hybridize to a 3′ (indicated by “3P”) or to a 5′ (indicated by “5P”) sequence of a cellular microRNA molecule. In recombinant RNA molecules as used in the working examples described hereinbelow, recombinant RNA molecules included recognition sequences corresponding to the 5′ (5P) sequences from Table 3, though skilled persons would appreciate that recombinant RNA molecules in accordance with the present disclosure could include recognition sequences corresponding to 3′ (3P) recognition sequences from Table 3 also or instead. Skilled persons would also appreciate that a miR recognition sequence may differ recombinant RNA molecules

TABLE 3
Non-limiting examples of human miR
recognition nucleotide sequences

| SEQ | | Recognition sequence |
|---|---|---|
| ID NO | miR | nucleotide sequence |
| 7 | 1-5P | AUGGGCAUAUAAAGAAGUAUGU |
| 8 | 1-3P | AUGGGCAUAUAAAGAAGUAUGU |
| 9 | 195a-5P | GCCAAUAUUUCUGUGCUGCUA |
| 10 | 195a-3P | GCCAAUAUUUCUGUGCUGCUA |
| 11 | 200c-5P | CCAAACACUGCUGGGUAAGACG |
| 12 | 200c-3P | UCCAUCAUUACCCGGCAGUAUUA |
| 13 | 208-5P | GUAUAACCCGGGCCAAAAGCUC |
| 14 | 208-3P | CCUGGCCGUGUGGUUAGUGAUU |
| 15 | LeU7f-5P | AACUAUACAAUCUACUACCUCA |
| 16 | LeU7f-3P | GGGAAGGCAAUAGAUUGUAUAG |
| 17 | 143-5P | ACCAGAGAUGCAGCACUGCACC |
| 18 | 143-3P | GAGCUACAGUGCUUCAUCUCA |
| 19 | 222-5P | AGGAUCUACACUGGCUACUGAG |
| 20 | 222-3P | ACCCAGUAGCCAGAUGUAGCU |
| 21 | 142a-5P | AGUAGUGCUUUCUACUUUAUG |
| 22 | 142a-3P | UCCAUAAAGUAGGAAACACUACA |
| 23 | 122-5P | CAAACACCAUUGUCACACUCCA |
| 24 | 122-3P | UAUUUAGUGUGAUAAUGGCGUU |
| 25 | 146a-5P | AACCCAUGGAAUUCAGUUCUCA |
| 26 | 146a-3P | CUGAAGAACUGAAUUUCAGAGG |
| 27 | 34c-5P | GCAAUCAGCUAACUACACUGCCU |
| 28 | 34c-3P | CCUGGCCGUGUGGUUAGUGAUU |
| 29 | 17-5P | CUACCUGCACUGUAAGCACUUUG |
| 30 | 17-3P | CUACAAGUGCCUUCACUGCAGU |
| 31 | 125-5P | UCACAAGUUAGGGUCUCAGGGA |
| 32 | 125-3P | AGCUCCCAAGAGCCUAACCCGU |
| 33 | 26a-5P | AGCCUAUCCUGGAUUACUUGAA |
| 34 | 26a-3P | GAAACAAGUAAUCAAGAAUAGG |
| 35 | 92a-5P | AGCAUUGCAACCGAUCCCAACCU |
| 36 | 92a-3P | ACAGGCCGGGACAAGUGCAAUA |
| 37 | 20a-5P | CUACCUGCACUAUAAGCACUUUA |
| 38 | 20a-3P | CUUUAAGUGCUCAUAAUGCAGU |
| 39 | 486a-5P | CUCGGGGCAGCUCAGUACAGGA |
| 40 | 486a-3P | AUCCUGUACUGAGCUGCCCCG |

In accordance with the present disclosure, a miR recognition sequence for a cellular microRNA identified herein may differ from a miR recognition sequence as set out in Table 3, provided said cellular microRNA may bind to said recognition sequence and reduce translation from a recombinant RNA molecule including the miR recognition sequence. In some cases, a recognition sequence may be longer or shorter that a sequence identified in Table 3, and in some cases may include a substitution for one, two, three, four, five, six, seven, or more nucleotides in a sequence of Table 3.

modRNA

A nucleoside is a molecule including a nitrogenous base (i.e., a nucleobase) linked to a pentose (e.g., deoxyribose or ribose) sugar. Nitrogenous bases which form nucleosides include adenine, guanine, cytosine, 5-methyl cytosine, uracil, and thymine. Suitable ribonucleosides (which comprise ribose as the pentose sugar) include, e.g., adenosine (A), guanosine (G), 5-methyluridine (m5U), uridine (U), and cytidine (C). Nucleotides are molecules including a nucleoside (e.g., a ribonucleoside) and a phosphate group. Ribonucleotides include, e.g., adenosine monophosphate, adenosine diphosphate, adenosine triphosphate, guanosine monophosphate, guanosine diphosphate, guanosine triphosphate, cytidine monophosphate, cytidine diphosphate, cytidine triphosphate, uridine monophosphate, uridine diphosphate, uridine triphosphate, and derivatives thereof.

Modified RNA, or modRNA, is a synthetic modified RNA that can be used for expression of a gene of interest. Chemical modifications to a ribonucleotide included in modRNA may stabilize an RNA molecule, blunt an immune response, or enhance transcription. Additionally, unlike delivery of protein agents directly to a cell, which can activate the immune system, the delivery of modRNA can be achieved without immune impact. For example, substitution of uridine and cytidine with pseudouridine or N1-methylpseudouridine and 5-methylcytidine, respectively, drastically reduces the immune response elicited from exogenous RNA without such substitutions. Stability and translational efficiency from an RNA molecule may also be increased by including a 3′-O-Me-m7G(5′)ppp(5′)G Anti Reverse Cap Analog (ARCA) at the 5′ end of the RNA molecule.

modRNA may encompass an RNA molecule with at least uridine substituted with pseudouridine. modRNA may encompass an RNA molecule with at least cytidine substituted with 5-methylcytidine. modRNA may encompass an RNA molecule including the modified nucleoside 5-methylcytidine (5mC). modRNA may encompass an RNA molecule including the modified nucleoside 2-Thiouridine-5′-Triphosphate (2-thio ψU). modRNA may encompass an RNA molecule with at least the modified nucleosidel-Methylpseudouridine-5′-Triphosphate (1-mψU). modRNA may encompass an RNA molecule with at least the modified nucleoside N1-methyl-pseudouridine (NlmΨ) substituted for uridine. modRNA may encompass an RNA molecule wherein at least 5′ triphosphates are removed. modRNA may encompass an RNA molecule wherein at least a 3′-O-Me-m7G(5′)ppp(5′)G Anti Reverse Cap Analog (ARCA) cap or C₃₂H₄₃N₁₅O₂₄P₄ CleanCap Reagent AG is included in a 5′ untranslated regions of the RNA molecule.

modRNAs may be prepared by in vitro transcription. modRNA may be in vitro transcribed, e.g., from a linear DNA template using one or more reagents selected from a cap analog, guanosine triphosphate, adenosine triphosphate, cytidine triphosphate, uridine triphosphate, and derivatives thereof. A cap analog may be selected from Anti-Reverse Cap Analog (ARCA) 3′-O-Me-m7G(5′)ppp(5′)G, standard cap analog m7G(5′)ppp(5′)G, unmethylated cap analog G(5′)ppp(5′)G, methylated cap analog for A+1 sites m7G(5′)ppp(5′)A, and unmethylated cap analog for A+1 sites G(5′)ppp(5′)A. In certain examples, a cap analog is Anti-Reverse Cap Analog (ARCA) 3′-O-Me-m7G(5′)ppp(5′)G. According to some examples, modRNA may be in vitro transcribed from a plasmid template using one or more reagents selected from 3′-O-Me-m7G(5′)ppp(5′)G, guanosine triphosphate, adenosine triphosphate, cytidine triphosphate, N1-methylpseudouridine-5-triphosphate, and any one or more of the aforementioned examples of modRNA, or others, without limitation and in any combination.

Additional suitable modifications to a modRNA or mRNA molecule are well known in the art (see, e.g., U.S. Pat. No. 8,278,036 to Kariko et al.; U.S. Pat. No. 10,086,043 to Chien et al.; U.S. Patent Application Publication No. 2019/0203226 to Zangi et al.; and U.S. Patent Application Publication No. 2018/0353618 to Burkhardt et al., which are hereby incorporated by reference in their entirety). In some embodiments, the nucleoside that is modified in the modRNA is a uridine (U), a cytidine (C), an adenine (A), or guanine (G). The modified nucleoside can be, for example, m⁵C (5-methylcytidine), m⁶A (N⁶-methyladenosine), s²U (2-thiouridien), ψ (pseudouridine), or Um (2-O-methyluridine). Some exemplary chemical modifications of nucleosides in the modRNA molecule may further include, for example and without limitation, pyridine-4-one ribonucleoside, 5-aza-uridine, 2-thio-5-aza uridine, 2-thiouridine, 4-thio pseudouridine, 2-thio pseudouridine, 5-hydroxyuridine, 3-methyluridine, 5-carboxymethyl uridine, 1-carboxymethyl pseudouridine, 5-propynyl uridine, 1-propynyl pseudouridine, 5-taurinomethyluridine, 1-taurinomethyl pseudouridine, 5-taurinomethyl-2-thio uridine, 1-taurinomethyl-4-thio uridine, 5-methyl uridine, 1-methyl pseudouridine, 4-thio-1-methyl pseudouridine, 2-thio-1-methyl pseudouridine, 1-methyl-1-deaza pseudouridine, 2-thio-1-methyl-1-deaza pseudouridine, dihydrouridine, dihydropseudouridine, 2-thio dihydrouridine, 2-thio dihydropseudouridine, 2-methoxyuridine, 2-methoxy-4-thio uridine, 4-methoxy pseudouridine, 4-methoxy-2-thio pseudouridine, 5-aza cytidine, pseudoisocytidine, 3-methyl cytidine, N4-acetylcytidine, 5-formylcytidine, N4-methylcytidine, 5-hydroxymethylcytidine, 1-methyl pseudoisocytidine, pyrrolo-cytidine, pyrrolo-pseudoisocytidine, 2-thio cytidine, 2-thio-5-methyl cytidine, 4-thio pseudoisocytidine, 4-thio-1-methyl pseudoisocytidine, 4-thio-1-methyl-1-deaza pseudoisocytidine, 1-methyl-1-deaza pseudoisocytidine, zebularine, 5-aza zebularine, 5-methyl zebularine, 5-aza-2-thio zebularine, 2-thio zebularine, 2-methoxy cytidine, 2-methoxy-5-methyl cytidine, 4-methoxy pseudoisocytidine, 4-methoxy-1-methyl pseudoisocytidine, 2-aminopurine, 2,6-diaminopurine, 7-deaza adenine, 7-deaza-8-aza adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N⁶-methyladenosine, N⁶-isopentenyladenosine, N⁶-(cis-hydroxyisopentenyl) adenosine, 2-methylthio-N⁶-(cis-hydroxyisopentenyl) adenosine, N⁶-glycinylcarbamoyladenosine, N⁶-threonylcarbamoyladenosine, 2-methylthio-N⁶-threonyl carbamoyladenosine, N⁶,N⁶-dimethyladenosine, 7-methyladenine, 2-methylthio adenine, 2-methoxy adenine, inosine, 1-methyl inosine, wyosine, wybutosine, 7-deaza guanosine, 7-deaza-8-aza guanosine, 6-thio guanosine, 6-thio-7-deaza guanosine, 6-thio-7-deaza-8-aza guanosine, 7-methyl guanosine, 6-thio-7-methyl guanosine, 7-methylinosine, 6-methoxy guanosine, 1-methylguanosine, N²-methylguanosine, N²,N²-dimethylguanosine, 8-oxo guanosine, 7-methyl-8-oxo guanosine, 1-methyl-6-thio guanosine, N²-methyl-6-thio guanosine, or N²,N²-dimethyl-6-thio guanosine.

In an example, modifications made to the modRNA are independently selected from 5-methylcytosine, pseudouridine, and 1-methylpseudouridine.

In some embodiments, the modRNA comprises a modified uracil selected from the group consisting of pseudouridine (W), pyridine-4-one ribonucleoside, 5-aza uridine, 6-aza uridine, 2-thio-5-aza uridine, 2-thio uridine (s2U), 4-thio uridine (s4U), 4-thio pseudouridine, 2-thio pseudouridine, 5-hydroxy uridine (ho⁵U), 5-aminoallyl uridine, 5-halo uridine (e.g., 5-iodom uridine or 5-bromo uridine), 3-methyl uridine (m³U), 5-methoxy uridine (mo⁵U), uridine 5-oxyacetic acid (cmoSU), uridine 5-oxyacetic acid methyl ester (mcmoSU), 5-carboxymethyl uridine (cm⁵U), 1-carboxymethyl pseudouridine, 5-carboxyhydroxymethyl uridine (chm⁵U), 5-carboxyhydroxym ethyl uridine methyl ester (mchm⁵U), 5-methoxycarbonylmethyl uridine (mcm⁵U), 5-methoxycarbonylmethyl-2-thio uridine (mcm⁵s2U), 5-aminomethyl-2-thio uridine (nm⁵s2U), 5-methylaminomethyl uridine (mnm⁵U), 5-methylaminomethyl-2-thio uridine (mnm⁵s2U), 5-methylaminomethyl-2-seleno uridine (mnm⁵se²U), 5-carbamoylmethyl uridine (ncm⁵U), 5-carboxymethylaminomethyl uridine (cmnm⁵U), 5-carboxymethylaminomethyl-2-thio uridine (cmnm⁵s2U), 5-propynyl uridine, 1-propynyl pseudouridine, 5-taurinomethyl uridine (TCm⁵U), 1-taurinomethyl pseudouridine, 5-taurinomethyl-2-thio uridine (^{TM}^{⁵}s2U), 1-taurinomethyl-4-thio pseudouridine, 5-methyl uridine (m⁵U, e.g., having the nucleobase deoxythymine), 1-methyl pseudouridine (m¹ψ), 5-methyl-2-thio uridine (m⁵s2U), 1-methyl-4-thio pseudouridine (m₁s⁴ψ), 4-thio-1-methyl pseudouridine, 3-methyl pseudouridine (m³ψ), 2-thio-1-methyl pseudouridine, 1-methyl-1-deaza pseudouridine, 2-thio-1-methyl-1-deaza pseudouridine, dihydrouridine (D), dihydropseudouridine, 5,6-dihydrouridine, 5-methyl dihydrouridine (m⁵D), 2-thio dihydrouridine, 2-thio dihydropseudouridine, 2-methoxy uridine, 2-methoxy-4-thio uridine, 4-methoxy pseudouridine, 4-methoxy-2-thio pseudouridine, N¹-methyl pseudouridine, 3-(3-amino-3-carboxypropyl) uridine (acp³U), 1-methyl-3-(3-amino-3-carboxypropyl) pseudouridine (acp³ψ), 5-(isopentenylaminomethyl) uridine (inm⁵U), 5-(isopentenylaminomethyl)-2-thio uridine (inm⁵s2U), α-thio uridine, 2′-O-methyl uridine (Um), 5,2′-O-dimethyl uridine (m⁵Um), 2′-O-methyl pseudouridine (ym), 2-thio-2′-O-methyl uridine (s2Um), 5-methoxycarbonylmethyl-2′-O-methyl uridine (mcm⁵Um), 5-carbamoylmethyl-2′-O-methyl uridine (ncm⁵Um), 5-carboxymethylaminomethyl-2′-O-methyl uridine (cmnm⁵Um), 3,2′-O-dimethyl uridine (m³Um), 5-(isopentenylaminomethyl)-2′-O-methyl uridine (inm⁵Um), 1-thio uridine, deoxythymidine, 2′-F-ara uridine, 2′-F uridine, 2′-OH-ara uridine, 5-(2-carbomethoxyvinyl) uridine, and 5-3-(1-E-propenylamino) uridine.

In some embodiments, the modRNA comprises a modified cytosine selected from the group consisting of 5-aza cytidine, 6-aza cytidine, pseudoisocytidine, 3-methyl cytidine (m³C), N⁴-acetyl cytidine (act), 5-formyl cytidine (f⁵C), N⁴-methyl cytidine (m⁴C), 5-methyl cytidine (m⁵C), 5-halo cytidine (e.g., 5-iodo cytidine), 5-hydroxymethyl cytidine (hm⁵C), 1-methyl pseudoisocytidine, pyrrolo-cytidine, pyrrolo-pseudoisocytidine, 2-thio cytidine (s2C), 2-thio-5-methyl cytidine, 4-thio pseudoisocytidine, 4-thio-1-methyl pseudoisocytidine, 4-thio-1-methyl-1-deaza pseudoisocytidine, 1-methyl-1-deaza pseudoisocytidine, zebularine, 5-aza zebularine, 5-methyl zebularine, 5-aza-2-thio zebularine, 2-thio zebularine, 2-methoxy cytidine, 2-methoxy-5-methyl cytidine, 4-methoxy pseudoisocytidine, 4-methoxy-1-methyl pseudoisocytidine, lysidine (k²C), alpha-thio cytidine, 2′-O-methyl cytidine (Cm), 5,2′-O-dimethyl cytidine (m⁵Cm), N⁴-acetyl-2′-O-methyl cytidine (ac⁴Cm), N⁴,2′-O-dimethyl cytidine (m⁴Cm), 5-formyl-2′-O-methyl cytidine (f⁵Cm), N⁴,N⁴,2′-O-trimethyl cytidine (m⁴²Cm), 1-thio cytidine, 2′-F-ara cytidine, 2′-F cytidine, and 2′-OH-ara cytidine.

In some embodiments, the modRNA comprises a modified adenine selected from the group consisting of 2-amino purine, 2,6-diamino purine, 2-amino-6-halo purine (e.g., 2-amino-6-chloro purine), 6-halo purine (e.g., 6-chloro purine), 2-amino-6-methyl purine, 8-azido adenosine, 7-deaza adenine, 7-deaza-8-aza adenine, 7-deaza-2-amino purine, 7-deaza-8-aza-2-amino purine, 7-deaza-2,6-diamino purine, 7-deaza-8-aza-2,6-diamino purine, 1-methyl adenosine (m¹A), 2-methyl adenine (m²A), N⁶-methyl adenosine (m⁶A), 2-methylthio-N⁶-methyl adenosine (ms²m⁶A), N⁶-isopentenyl adenosine (i⁶A), 2-methylthio-N⁶-isopentenyl adenosine (ms²i⁶A), N⁶-(cis-hydroxyisopentenyl) adenosine (io⁶A), 2-methylthio-N⁶-(cis-hydroxyisopentenyl) adenosine (ms²io⁶A), N⁶-glycinylcarbamoyl adenosine (g⁶A), N⁶-threonylcarbamoyl adenosine (t⁶A), N⁶-methyl-N⁶-threonylcarbamoyl adenosine (m⁶t6A), 2-methylthio-N⁶-threonylcarbamoyl adenosine (ms²g⁶A), N⁶,N⁶-dimethyl adenosine (m⁶₂A), N⁶-hydroxynorvalyIcarbamoyl adenosine (hn⁶A), 2-methylthio-N⁶-hydroxynorvalylcarbamoyl adenosine (ms²hn⁶A), N⁶-acetyl adenosine (ac⁶A), 7-methyl adenine, 2-methylthio adenine, 2-methoxy adenine, alpha-thio adenosine, 2′-O-methyl adenosine (Am), N⁶,2′-O-dimethyl adenosine (m⁶Am) N⁶,N⁶,2′-O-trimethyl adenosine (m⁶₂Am), 1,2′-O-dimethyl adenosine (m¹Am), 2′-O-ribosyl adenosine (phosphate) (Ar(p)), 2-amino-N⁶-methyl purine, 1-thio adenosine, 8-azido adenosine, 2′-F-ara adenosine, 2′-F adenosine, 2′-OH-ara adenosine, and N⁶-(19-amino-pentaoxanonadecyl) adenosine.

In some embodiments, the modRNA comprises a modified guanine selected from the group consisting of inosine (I), 1-methyl inosine (m¹I), wyosine (imG), methylwyosine (mimG), 4-demethyl wyosine (imG-14), isowyosine (imG2), wybutosine (yW), peroxywybutosine (o2yW), hydroxywybutosine (OHyW), undermodified hydroxywybutosine (OHyWy), 7-deaza guanosine, queuosine (Q), epoxyqueuosine (oQ), galactosyl queuosine (galQ), mannosyl queuosine (manQ), 7-cyano-7-deaza guanosine (preQ₀), 7-aminomethyl-7-deaza guanosine (preQ₁), archaeosine (G⁺), 7-deaza-8-aza guanosine, 6-thio guanosine, 6-thio-7-deaza guanosine, 6-thio-7-deaza-8-aza guanosine, 7-methyl guanosine (m⁷G), 6-thio-7-methyl guanosine, 7-methyl inosine, 6-methoxy guanosine, 1-methyl guanosine (m¹G), N²-methyl-guanosine (m²G), N²,N²-dimethyl guanosine (m²₂G), N^{2,7}-dimethyl guanosine (m^{2,7}G), N², N^{2,7}-dimethyl guanosine (m^{2,2,7}G), 8-oxo guanosine, 7-methyl-8-oxo guanosine, 1-methio guanosine, N²-methyl-6-thio guanosine, N²,N²-dimethyl-6-thio guanosine, alpha-thio guanosine, 2′-O-methyl guanosine (Gm), N²-methyl-2′-O-methyl guanosine (m²Gm), N²,N²-dimethyl-2′-O-methyl guanosine (m²₂Gm), 1-methyl-2′-O-methyl guanosine (m¹Gm), N^{2,7}-dimethyl-2′-O-methyl guanosine (m^{2,7}Gm), 2′-O-methyl inosine (1m), 1,2′-O-dimethyl inosine (m¹Im), 2′-O-ribosyl guanosine (phosphate) (Gr(p)), 1-thio guanosine, O⁶-methyl guanosine, 2′-F-ara guanosine, and 2′-F guanosine.

modRNA may include, for example, a non-natural or modified nucleotide. The non-natural or modified nucleotide may include, for example, a backbone modification, sugar modification, or base modification. The non-natural or modified nucleotide may include, for example, a base modification. In some embodiments, the base modification is selected from the group consisting of 2-amino-6-chloropurine riboside 5′ triphosphate, 2-aminoadenosine 5′ triphosphate, 2-thiocytidine 5′ triphosphate, 2-thiouridine 5′ triphosphate, 4-thiouridine 5′ triphosphate, 5-aminoallylcytidine 5′ triphosphate, 5-aminoallyluridine 5′ triphosphate, 5-bromocytidine 5′ triphosphate, 5-bromouridine 5′ triphosphate, 5-iodocytidine 5′ triphosphate, 5-iodouridine 5′ triphosphate, 5-methylcytidine 5′ triphosphate, 5-methyluridine 5′ triphosphate, 6-azacytidine 5′ triphosphate, 6-azauridine 5′ triphosphate, 6-chloropurine riboside 5′-triphosphate, 7-deazaadenosine 5′ triphosphate, 7-deazaguanosine 5′ triphosphate, 8-azaadenosine 5′ triphosphate, 8-azidoadenosine 5′ triphosphate, benzimidazole riboside 5′ triphosphate, N¹-methyladenosine 5′ triphosphate, N¹-methylguanosine 5′ triphosphate, N⁶-methyladenosine 5′ triphosphate, O⁶-methylguanosine 5′ triphosphate, N¹-methyl-pseudouridine 5′ triphosphate, puromycin 5′-triphosphate, and xanthosine 5′ triphosphate. Thus, according to some embodiments, the modRNA comprises N¹-methyl-pseudouridine 5′ triphosphate.

Nanoparticles

A nanoparticle is a composition of matter having a nanoscale-dimension size, such as a diameter from about 1 nm t about 100 nm, though may refer to compositions having a larger diameter as well, such as up to 500 nm. A nanoparticle may provide enhanced cellular uptake and stability of a first and second RNA molecule as described herein. Packaging a first and second RNA molecule in a nanoparticle may protect them from extracellular degradation processes that may otherwise occur following, for example, systemic or other administration of a first and second RNA molecule, thereby increasing cellular uptake by prolonging the time period between administration of the first and second RNA molecule and when they are taken up by a cell.

A nanoparticle may also improve cellular uptake by providing a mechanism for cellular entry, such as fusion of a nanoparticle's membrane with a cellular membrane for delivery of the nanoparticle's payload to an intracellular compartment. A variety of materials are known to be suitable for nanoparticles for intracellular delivery of their payloads such as lipid or phospholipid micelles or liposomes, metal nanoparticles, such as gold, aluminum, iron nanoparticles, polyacrylamide, polyacrylate, or chitosan nanoparticles, a polymer-based nanoparticle such as a poly lactic-co-glycolic nanoparticle, may be used in accordance with the present disclosure, with a first and second RNA molecule packaged in any type of nanoparticle suitable for an intended purpose, synthesized according to standard methods.

Target Cell and Off-Target Cell

A target cell may include a cell in which expression of a gene of interest included in an expression regulatory system as described herein may be desired. A target cell may be a cell in culture, such as any immortalized cell line, or any tumor cell line, or any other cell line that may be maintained in culture, such as a genetically modified or characterized culture cell line that may be a model for a tissue type, a disease state, or a system for testing responses to pharmacological or other agents. A target cell may be an ex vivo cell, originating from cells or tissue harvested from an organism or other living source and cultured or maintained in vitro or in any model system for maintaining ex vivo cells. In an example, ex vivo cells as target cells may have been harvested from a genetically modified organism.

A target cell may be a cell within an organism. A target cell may be a cell identified by a type of tissue in which it is found or which it makes up. A target cell may be a cell within a tissue of an organ, or any cell found in or identified by an organ in which it is located. In an example, a target cell may be a non-diseased cell. In an example, a target cell may be a diseased cell, malfunctioning cell, tumor cell, senescing cell, or other cell type selected or identified by its status within an organ, tissue, or organism. A target cell may have originated within an organ in which it is found, or may have been generated in one organ then traveled through the body and later located in another organ. A target cell may be an implanted cell, which originated outside the body and was implanted or injected within the body. A target cell may be autologous, such as an implanted cell that had been harvested from the implant recipient before being implanted back into the recipient, or may be allogenic, such as an implanted cell that had been harvested from a donor other than the implant recipient before being implanted into the recipient. In an example, a target cell may be an implanted cell wherein the implanted cell includes one or more recombinant genetic modification. In an example, a target cell may be an ex vivo cell which is implanted after being transfected with a first and second RNA molecule of an expression regulatory system as disclosed herein.

A first RNA molecule of an expression regulatory system as disclosed herein may encode a translation suppressor and include one or more recognition sequence for one or more first miR wherein said first miR is expressed in a target cell. As a non-limiting example, a target cell may be a heart tissue cell, such as a cardiomyocyte. A first miR recognition sequence of a first RNA molecule of an expression regulatory system wherein a heart tissue cell, such as a cardiomyocyte, is a target cell, may include a recognition sequence for one or both of miR-1 and miR-208. As a non-limiting example, a target cell may be a tumor cell, such as a breast tumor cell or other tumor cell type. A first miR recognition sequence of a first RNA molecule of an expression regulatory system wherein a tumor cell, such as a breast tumor cell, is a target cell, may include a recognition sequence for one or more of miR-155, miR-10b, miR-181a, miR-181b, and any combination of two or more thereof. As a non-limiting example, a target cell may be a lung tissue cell, such as a pulmonary myofibroblast or other lung tissue cell type. A first miR recognition sequence of a first RNA molecule of an expression regulatory system wherein a lung tissue cell, such as a pulmonary myofibroblast, is a target cell, may include a recognition sequence for miR-146a. As a non-limiting example, a target cell may be a spleen tissue cell or a bone marrow tissue cell, such as a monocyte or other spleen tissue cell type or bone marrow tissue cell type. A first miR recognition sequence of a first RNA molecule of an expression regulatory system wherein a spleen tissue cell or a bone marrow tissue cell, such as a monocyte, may include a recognition sequence for one or more of miR-20, miR-148, miR-223, or any combination of two or more of the foregoing.

An off-target cell may include any cell in which expression of a gene of interest of an expression regulatory system as disclosed herein may be undesirable or otherwise not preferred. An off-target cell may be any cell other than a target cell. An off-target cell may be a cell of an organ or tissue other than the organ of tissue of a target cell, or of a cell type different from a cell type of a target cell but which may be within the same organ or tissue as a target cell. An off-target cell may be a cell within an organism. An off-target cell may be a cell identified by a type of tissue in which it is found or which it makes up. An off-target cell may be a cell within a tissue of an organ, or any cell found in or identified by an organ in which it is located. In an example, an off-target cell may be a non-diseased cell. In an example, an off-target cell may be a diseased cell, malfunctioning cell, tumor cell, senescing cell, or other cell type selected or identified by its status within an organ, tissue, or organism. An off-target cell may have originated within an organ in which it is found, or may have been generated in one organ then traveled through the body and later located in another organ. An off-target cell may be an implanted cell, which originated outside the body and was implanted or injected within the body. An off-target cell may be autologous, such as an implanted cell that had been harvested from the implant recipient before being implanted back into the recipient, or may be allogenic, such as an implanted cell that had been harvested from a donor other than the implant recipient before being implanted into the recipient. In an example, an off-target cell may be an implanted cell wherein the implanted cell includes one or more recombinant genetic modification. In an example, an off-target cell may be an ex vivo cell which is implanted after being transfected with a first and second RNA molecule of an expression regulatory system as disclosed herein.

A first RNA molecule of an expression regulatory system as disclosed herein may encode a translation suppressor and include one or more recognition sequence for one or more first miR wherein said first miR is not expressed in one or more off-target cell type, or in which expression of said first miR dos not suppress translation of expression from said first RNA molecule in one or more off-target cell or may do so but to less of a degree than it may in a target cell.

As a non-limiting example, a target cell may be a heart tissue cell, such as a cardiomyocyte. A first miR recognition sequence of a first RNA molecule of an expression regulatory system wherein a heart tissue cell, such as a cardiomyocyte, is a target cell, may include, as a non-limiting example, a recognition sequence for one or both of miR-1 and miR-208. As a non-limiting example, a target cell may be a tumor cell, such as a breast tumor cell or other tumor cell type. A first miR recognition sequence of a first RNA molecule of an expression regulatory system wherein a tumor cell, such as a breast tumor cell, is a target cell, may include, as a non-limiting example, a recognition sequence for one or more of miR-155, miR-10b, miR-181a, miR-181b, and any combination of two or more thereof. As a non-limiting example, a target cell may be a lung tissue cell, such as a pulmonary myofibroblast or other lung tissue cell type. A first miR recognition sequence of a first RNA molecule of an expression regulatory system wherein a lung tissue cell, such as a pulmonary myofibroblast, is a target cell, may include, as a non-limiting example, a recognition sequence for miR-146a. As a non-limiting example, a target cell may be a spleen tissue cell or a bone marrow tissue cell, such as a monocyte or other spleen tissue cell type or bone marrow tissue cell type. A first miR recognition sequence of a first RNA molecule of an expression regulatory system wherein a spleen tissue cell or a bone marrow tissue cell, such as a monocyte, is a target cell may include, as a non-limiting example, a recognition sequence for one or more of miR-20, miR-148, miR-223, or any combination of two or more of the foregoing.

In an example, a second RNA molecule of an expression regulatory system as disclosed herein may encode a gene of interest and include one or more recognition sequence for one or more second miR wherein said second miR is expressed in an off-target cell. As a non-limiting example, a target cell may be a heart tissue cell, such as a cardiomyocyte. A second miR recognition sequence of a second RNA molecule of an expression regulatory system wherein a heart tissue cell, such as a cardiomyocyte, is a target cell, may include, as non-limiting example, a recognition sequence for one or both of miR-143 and miR-146a. As a non-limiting example, a target cell may be a tumor cell, such as a breast tumor cell or other tumor cell type. A second miR recognition sequence of a second RNA molecule of an expression regulatory system wherein a tumor cell, such as a breast tumor cell, is a target cell, may include, as non-limiting example, a recognition sequence for one or both of miR-143 and miR-122. As a non-limiting example, a target cell may be a lung tissue cell, such as a pulmonary myofibroblast or other lung tissue cell type. A second miR recognition sequence of a second RNA molecule of an expression regulatory system wherein a lung tissue cell, such as a pulmonary myofibroblast, is a target cell, may include, as a non-limiting example, a recognition sequence for miR-143. As a non-limiting example, a target cell may be a spleen tissue cell or a bone marrow tissue cell, such as a monocyte or other spleen tissue cell type or bone marrow tissue cell type. A second miR recognition sequence of a second RNA molecule of an expression regulatory system wherein a spleen tissue cell or a bone marrow tissue cell, such as a monocyte, is a target cell may include, as a non-limiting example, a recognition sequence for miR-122.

In an example, where an expression regulatory system as disclosed herein includes a nanoparticle, a charge of the nanoparticle may relate to cellular uptake of the first and second RNA molecules of the expression regulatory system. For example, a charge or lack thereof of an expression regulatory system including a nanoparticle may affect whether and to what degree an organ, tissue, or cell may uptake the RNA. For example, systemic injection with positive charged nanoparticles may promote uptake in lung and heart tissue, whereas negative nanoparticles may promote uptake by spleen tissue, and whereas neutral charged nanoparticles may promote uptake by liver tissue. Contributors of charge to a nanoparticle may include, for example, negative charge imparted by a nucleotide such as an RNA molecule, and positive charge imparted by, for example, lipid molecules (e.g., increasing particle size by adding more lipid during synthesis may increase relative positive or decrease relative negative charge). Other charge-carrying components may also be included in a nanoparticle (e.g., a polymers, such as polyethyleneimine or another charged polymer, a peptide, etc.), bearing a positive or negative charge, and in different relative amounts so as to affect overall nanoparticle charge. Charge of a nanoparticle may be affected by method of synthesis, wherein ratio of RNA (or other negatively charged nanoparticle constituent) to lipid (or other positively charged constituent) may be increased for decreasing positive charge or increasing negative charge of produced nanoparticles, relative amounts of RNA (or other negatively charged nanoparticle constituent) to lipid (or other positively charged constituent) may be decreased for increasing positive charge or decreasing negative charge of produced nanoparticles, and relative amounts of RNA (or other negatively charged nanoparticle constituent) to lipid (or other positively charged constituent) may be modified so as to produce nanoparticles with relatively neutral charge.

Gene of Interest

A gene of interest may be any transcript whose expression in a cell is desired. A gene of interest may include a full coding sequence for a protein, including an initiation codon and a stop codon and a series of nucleotides therebetween encoding a protein's amino acid sequence, as well as a 5′ UTR sequence or 3′ UTR sequence, and any additional cis-acting factors to enable translation of the RNA to a protein product, as well as to enhance stability of the RNA (other than as may be involved in the suppression of translation as may be desired in an off-target cell according to the present disclosure, as described above). A gene of interest may encode a peptide or protein having essentially any desired amino acid sequence, as would be appreciated by skilled persons, including without limitation a structural protein, an enzyme, an intracellular protein, and extracellular protein, a nuclear protein, a signaling protein, a secreted protein, or any naturally occurring or synthetic protein that includes features of any one or more of the foregoing attributes.

In an example, a gene of interest may encode a constitutively active protein, which shares structural features with an active protein such as an enzyme except that it may lack negative regulatory elements that function to inhibit or prevent activity of the active protein unless acted upon by another factor such as a signaling molecule, kinase, proximity to a structural cellular feature, etc. Or a gene of interest may encode a dominant negative protein, which may inhibit activity endogenously expressed by a target cell such as by binding to it or sequestering its binding partners by binding to them and thereby preventing the endogenous protein form performing its normal function. In an example, a gene of interest may encode a protein also encoded for by the genome of a target cell but perhaps not endogenously expressed in a target cell, or expressed at a lower level than desired. In an example, a gene of interest may encode a therapeutic protein. whose expression in a target cell is intended to confer a therapeutic benefit. For example, a gene of interest may encode a protein whose low or lack of expression in a target cell may be believed to correspond to an disease state or other pathological condition, such that increasing expression the such protein in a target cell may treat such disease or pathological condition. Or, an aberrant or disease-associated variant of a protein may be expressed in a target cell and a gene of interest may encode a different variant of such protein that substitutes for the endogenous variant.

Several possible genes of interest for inclusion in an expression regulatory system are disclosed herein, including in the following examples. Some may be considered illustrative examples, disclosed herein as demonstrating target cell expression driven by an expression regulatory system and types of uses of such a system. Such examples should not be considered as limiting genes of interest that may be included within an expression regulatory system as disclosed herein, which may include other genes of interest.

In an example, a gene of interest may encode a cell cycle inducer protein. For example, a target cell may be a heart tissue cell, such as a cardiomyocyte. Expression of a cell cycle inducer protein following uptake of an expression regulatory system as disclosed herein may promote cardiomyocyte growth and promote beneficial cardiac remodeling following heart injury such as a cardiac ischemic event. Cell cycle inducer proteins may include, without limitation, Lin28, Pyruvate Kinase Muscle Isozyme M2 (Pkm2), 0-catenin, caERBB2, Yes Associated Protein 1 (YAP), Cyclin D1, and c-Myc.

Lin28 is a suppressor of Let7 that controls cell cycle regulators Treatment of cardiomyocytes post-myocardial infarction using modRNA constructs encoding Lin28 induces cardiomyocyte proliferation, reduce apoptosis, and increase capillary density.

Pyruvate Kinase Muscle Isozyme M2 (Pkm2) is a pro-proliferative factor, highly expressed in regenerative fetal and early neonatal cardiomyocytes. In the cytoplasm, Pkm2 shifts the metabolic fate from glycolysis to pentose phosphate pathway (“PPP”) by reducing the conversion of phosphoenolpyruvate to pyruvate, which leads to the accumulation of galactose, a glycolysis intermediate, and activation of PPP via Glucose-6-phosphate dehydrogenase (G6pd). PPP pathway activation leads to the synthesis of nucleotides, amino acids, and lipids and the production of reduced NADPH, increase nitric oxide synthase and DNA repair In the nucleus, Pkm2 directly interacts with the transcription factors μ-catenin and Hif1α. This interaction promotes the expression of genes such as in Ccdn1, c-Myc and Vegfa, and Bcl2. Restoration of Pkm2 levels using modRNA into adult cardiomyocytes post-myocardial infarction significantly and exclusively induces cardiomyocyte proliferation; associated with improved cardiac function, reduced scar size, and increased heart to body weight ratio; reduce cardiomyocyte size; reduce apoptosis; and increase capillary density.

β-catenin is a subunit of the cadherin protein complex and acts as an intracellular signal transducer in the Wnt signaling pathway. In cardiac muscle, β-catenin localizes to adherens junctions in intercalated disc structures, which are critical for electrical and mechanical coupling between adjacent cardiomyocytes. Loss of β-catenin during early heart formation results in multiple heart defects and lethality demonstrating its crucial function for embryonic heart development. In adults, β-catenin signaling plays an important role in normal and stress-induced cardiac hypertrophic remodeling. Wnt/β-catenin signaling may function in a stage-specific biphasic manner, either promoting or inhibiting cardiogenesis.

ERBB2 (erb-b2 receptor tyrosine kinase 2) forms a heterodimer with other epidermal growth factor receptor tyrosine kinase family members. ERBB2 is required for cardiomyocyte proliferation at embryonic/neonatal stages. Transient induction of a constitutively active ERBB2 (caERBB2) for 10-20 days after ischemic injury, either in juvenile or adult hearts, has been shown to trigger a series of events starting with cardiomyocyte dedifferentiation, proliferation, neovascularization and, after ERBB2-signaling termination, proceeding to cardiomyocyte re-differentiation that together lead to anatomical and functional heart regeneration.

Yes Associated Protein 1 (YAP) is a transcriptional coactivator, whose activation in adult cardiomyocytes has been shown to increases cardiomyocyte proliferation and improve cardiac function after myocardial infarction in mice.

Cyclin D1 is a regulatory subunit of CDK4 and CDK6, whose activity is required for cell cycle G1/S transition. Overexpression of cyclin D1 results in an increase in CDK4 levels in the adult myocardium, as well as modest increases in proliferating cell nuclear antigen and CDK2 levels. Expression of cyclin D1 promotes cell cycle reentry of cardiomyocytes in adult hearts.

cMyc is highly expressed in fetal, proliferating cardiac myocytes. Although expressed at low levels in the adult heart under normal physiological conditions, c-Myc expression is rapidly upregulated in response to hypertrophic stimuli. Activation of cMyc in adult myocardium provokes cell cycle reentry in post-mitotic myocytes.

In another example, a gene of interest included in an expression regulatory system as disclosed herein may include one or more of a cardiac reprogramming gene and a reprogramming helper gene. Examples of cardiac reprogramming genes or proteins they encode include GATA Binding Protein 4 (Gata4), Myocyte Enhancer Factor 2C (Mef2c), T-box 5 (Tbx), and Heart- and neural crest derivatives-expressed protein 2 (Hand2). Examples of cardiac reprogramming helper genes or proteins they encode include, Dominant Negative (DN) transforming growth factor beta (DN-TGFβ), DN-Wingless-related integration site 8a (DN-Wnt8a), and Acid ceramidase (AC). Uptake by heart tissue cells of an expression regulatory system including, for example, one or more of the foregoing cardiac reprogramming genes or reprogramming helper genes as gene of interest may promote cardiac regeneration, remodeling, and function following an insult such as a cardiac ischemic event.

In another non-limiting example of an expression regulatory system as disclosed herein, a gene of interest may encode type 2 phosphatidylinositol-5-phosphate 4-kinase gamma (pip4k2c, used herein to refer to a polynucleotide coding for the protein phosphatidylinositol-5-phosphate 4-kinase type 2 gamma (PI5P4Kγ). Pip4k2c is a type 2 phosphatidylinositol-5-phosphate 4-kinase (PI5P4K), which converts phosphatidylinositol-5-phosphate to phosphatidylinositol 4,5-bisphosphate in mammals. The mammalian gene PI5P4K encodes for three enzymes—PI5P4Kα, PI5P4Kβ, and PI5P4Kγ. Pip4k2c inhibits mTORC1-signaling. The mTORC1 signaling pathway is one of the main signaling pathways that induce cardiac hypertrophy after pressure overload Moreover, TGF-β signaling plays an important role in the pathogenesis of cardiac fibrosis, and increased expression of Pip4k2c significantly attenuates and/or prevents cardiac hypertrophy and fibrosis in the failing heart and improved cardiac function via inhibition of mTORC1 and TGF-β activity. TGFβ1, is pro-fibrotic, increases after cardiac ischemic injury and can lead to cardiomyocyte cell death. As the TGFβ1 and, in some cases, mTORC1 pathways are crucial to other fibrotic diseases, such as pulmonary fibrosis and chronic renal fibrosis, uptake of an expression regulatory system including wherein a second RNA molecule includes a polynucleotide encoding phosphatidylinositol-5-phosphate 4-kinase type 2 gamma may be useful in treating such fibroses (e.g., if the expression regulatory system included miR recognition sequences compatible with promoting expression therefor in a lung tissue cell, such as a myofibroblast, as a target cell, or a heart tissue cell, or a renal tissue cell, as a cell of interest, as may be appropriate to an example of a fibrosis condition).

In another example, a gene of interest may encode an anti-apoptotic protein, a pro-apoptotic protein, a cell cycle-inducer protein, or cell-cycle arrest protein. In an example, a gene of interest may encode a p53 protein, a Herpes Simplex Virus type 1 thymidine kinase, a deltex protein, an E1A protein, a cystatin SA protein, a cystatin E/M protein, or a caspase 9 protein. In an example, a gene of interest may encode an antibody, an anti-angiogenic protein, or an angiogenic protein. In an example, a gene of interest may encode an anti-tumor protein. An anti-tumor protein may include a protein whose expression promotes apoptosis of cancerous cells, such as an apoptotic protein, or render tumor cells susceptible to a tumoricidal pharmacological treatment, or may otherwise promote cell death following expression in a tumor cell as a target cell.

In another non-limiting example, the gene of interest may encode a reporter protein or selection marker. Any reporting protein may be suitable. Non-limiting examples may include an antibiotic resistance marker, inactive human CD25 (ihCD25), a (3-galactosidase, or other selection marker or reporter protein.

In an example, a gene of interest may encode a reporter protein. The reporter protein may be a fluorescent protein. A fluorescent protein may include, without limitation, green fluorescent proteins (e.g., GFP, GFP-2, tagGFP, turboGFP, EGFP, Emerald, Azami Green, Monomeric Azami Green, CopGFP, AceGFP, ZsGreenl), yellow fluorescent proteins (e.g., YFP, EYFP, Citrine, Venus, YPet, PhiYFP, ZsYellowl), blue fluorescent proteins (e.g., EBFP, EBFP2, Azurite, mKalamal, GFPuv, Sapphire, T-sapphire), cyan fluorescent proteins (e.g., ECFP, Cerulean, CyPet, AmCyanl, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRedl, AsRed2, mRasberry, mStrawberry, Jred), and orange fluorescent proteins (mOrange, mKO, Kusabira-Orange, Monomeric Kusabira-Orange, mTangerine, tdTomato), or any other suitable fluorescent protein. In certain embodiments, the reporter protein is a fluorescent protein selected from the group consisting of green fluorescent protein (GFP), enhanced green fluorescent protein (EGFP), and yellow fluorescent protein (YFP).

In some embodiments, the reporter protein is luciferase. As used herein, the term “luciferase” refers to members of a class of enzymes that catalyze reactions that result in production of light. Luciferases have been identified in and cloned from a variety of organisms including fireflies, click beetles, sea pansy (Renilla), marine copepods, and bacteria among others. Examples of luciferases that may be used as reporter proteins include, e.g., Renilla (e.g., Renilla renformis) luciferase, Gaussia (e.g., Gaussia princeps) luciferase), Metridia luciferase, firefly (e.g., Photinus pyralis luciferase), click beetle (e.g., Pyrearinus termitilluminans) luciferase, deep sea shrimp (e.g., Oplophorus gracilirostris) luciferase). Luciferase reporter proteins include both naturally occurring proteins and engineered variants designed to have one or more altered properties relative to the naturally occurring protein, such as increased photostability, increased pH stability, increased fluorescence or light output, reduced tendency to dimerize, oligomerize, aggregate or be toxic to cells, an altered emission spectrum, and/or altered substrate utilization.

Administering an Expression Regulatory System to a Subject.

In an example, an expression regulatory system as disclosed herein may be administered to a subject. In a non-limiting example, the subject may have suffered a myocardial infarction or suffer from heart failure or other cardiac ischemic condition or insult. In another non-limiting example, the subject may suffer from cancer. In another non-limiting example, the subject may suffer from a fibrosis such as a cardiac fibrosis or a pulmonary fibrosis.

In an example, a gene of interest discussed above may be included in a second RNA molecule of an expression regulatory system and a first RNA molecule may include a recognition sequence for a miR that leads to translation of the gene of interest in a heart tissue cell as a target cell, such as a cardiomyocyte. In a further example, the second RNA molecule may also include one or more recognition sequence for a miR that decreases translation of the gene of interest in off-target cells, in accordance with aspects of the present disclosure. All combinations and permutations of miR recognition sequences for a heart tissue cell as a target cell and translation suppressors of a first RNA molecule, and translation suppressor recognition sequences and genes of interest disclosed herein as promoting cardiac regeneration or otherwise improving cardiac function, and including in some examples one or more recognition sequence to reduce translation in any off-target cell other than a heart tissue target cell as in the present example, disclosed in the present disclosure is explicitly incorporated in the present example.

In an example, a gene of interest discussed above may be included in a second RNA molecule of an expression regulatory system and a first RNA molecule may include a recognition sequence for a miR that leads to translation of the gene of interest in a tumor cell, such as a breast tumor cell, as a target cell. In a further example, the second RNA molecule may also include one or more recognition sequence for a miR that decreases translation of the gene of interest in off-target cells, in accordance with aspects of the present disclosure. All combinations and permutations of miR recognition sequences for a tumor cell as a target cell and translation suppressors of a first RNA molecule, and translation suppressor recognition sequences and genes of interest disclosed herein as an anti-tumor gene or pro-apoptotic gene, and including in some examples one or more recognition sequence to reduce translation in any off-target cell other than a tumor cell target cell as in the present example, disclosed in the present disclosure is explicitly incorporated in the present example.

In an example, a gene of interest discussed above may be included in a second RNA molecule of an expression regulatory system and a first RNA molecule may include a recognition sequence for a miR that leads to translation of the gene of interest in a lung tissue cell, such as a myofibroblast, as a target cell. In a further example, the second RNA molecule may also include one or more recognition sequence for a miR that decreases translation of the gene of interest in off-target cells, in accordance with aspects of the present disclosure. All combinations and permutations of miR recognition sequences for a lung tissue cell as a target cell and translation suppressors of a first RNA molecule, and translation suppressor recognition sequences and genes of interest disclosed herein as an anti-fibrosis gene, and including in some examples one or more recognition sequence to reduce translation in any off-target cell other than a tumor cell target cell as in the present example, disclosed in the present disclosure is explicitly incorporated in the present example.

In an example, a gene of interest discussed above may be included in a second RNA molecule of an expression regulatory system and a first RNA molecule may include a recognition sequence for a miR that leads to translation of the gene of interest in a bone marrow cell, or splenocyte, such as a monocyte, as a target cell. In a further example, the second RNA molecule may also include one or more recognition sequence for a miR that decreases translation of the gene of interest in off-target cells, in accordance with aspects of the present disclosure. All combinations and permutations of miR recognition sequences for a monocyte as a target cell and translation suppressors of a first RNA molecule, a gene of interest disclosed herein, and including in some examples one or more recognition sequence to reduce translation in any off-target cell other than a monocyte target cell as in the present example, disclosed in the present disclosure is explicitly incorporated in the present example.

In any of the foregoing example, any selection marker or reporter protein gene of interest may be included in an expression regulatory system as disclosed herein, or another known selection marker or reporter gene of interest. Any of the foregoing examples, without exception, may also include any one or more modRNA as disclosed herein as well, all combination and permutations of which are explicitly included herein.

In an example, an expression regulatory system may be administered to a subject by direct injection to an organ wherein a target cell may be located in a tissue of the organ. In another example, an expression regulatory system may be administered to a subject systemically, such as intravenously. In an example, an expression regulatory system administered intravenously may include a nanoparticle which, in some cases, may promote stability of a first and second RNA molecule or promote access to or uptake by a target cell. As further disclosed herein, in an example, a first, second, or both RNA molecules may include modRNA. modRNA may eliminate, reduce, prevent, or otherwise avoid stimulation of an immune response that may otherwise degrade RNA molecules of the expression regulatory system or reduce their access to cells or their efficiency in robustly promoting expression of a gene of interest. In an example, an expression regulatory system as disclosed herein may be administered repeatedly to a subject, without provoking an immune response or other untoward adverse health effects, including an example where the expression regulatory system include incorporation of one of more modRNA in one or both RNA molecule, or includes a nanoparticle that includes the RNA molecules, or both. For example, an expression regulatory system may be administered daily, or every two, three, four, or more days, or on repeated days separated by different directions from each other, depending on a desired frequency of administration or peak expression of a gene of interest.

Compositions of the present invention may be administered orally, parenterally, by inhalation, topically, rectally, nasally, buccally, sublingually, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, compositions may be administered orally, intraperitoneally or intravenously. Sterile injectable forms of compositions may be aqueous or oleaginous suspension. Suspensions may be formulated according to techniques known in the art using suitable dispersing or wetting agents and suspending agents. A sterile injectable preparation may also be a sterile injectable solution or suspension in a non-toxic parenterally acceptable diluent or solvent. Among acceptable vehicles and solvents that may be employed are water, Ringer's solution and isotonic sodium chloride solution. In addition, sterile, fixed oils may be employed as a solvent or suspending medium. Pharmaceutically acceptable compositions may be orally administered in any orally acceptable dosage form including capsules, tablets, aqueous suspensions or solutions.

An in vivo dosage unit (e.g., for contacting target cells within a subject) may include from, for example, 1 to 100 μg, 10 to 100 μg, 15 to 100 μg, 20 to 100 μg, 25 to 100 μg, and 1 to 200 μg (e.g., 1 μg, 2 μg, 3 μg, 4 μg, 5 μg, 6 μg, 7 μg, 8 μg, 9 μg, 10 μg, 11 μg, 12 μg, 13 μg, 14 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 110 μg, 120 μg, 130 μg, 140 μg, 150 μg, 160 μg, 170 μg, 180 μg, 190 μg, 200 μg of an expression regulatory system, including in an example a nanoparticle, as disclosed herein. In an example, a dosage unit may include, for example, 1 to 10 mg, 1 to 20 mg, 1 to 30 mg, 1 to 40 mg, 1 to 50 mg, 1 to 60 mg, 1 to 70 mg, 1 to 80 mg, 1 to 90 mg, 1 to 100 mg, 10 to 100 mg, 20 to 100 mg, 30 to 100 mg, 40 to 100 mg, 50 to 100 mg, 60 to 100 mg, 70 to 100 mg, 80 to 100 mg, and 90 to 100 mg (e.g., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg of an expression regulatory system, including in an example a nanoparticle, as disclosed herein.

Also included herein is administration of a first and second RNA molecule according to any of the foregoing examples to a subject for treatment for a medical condition or illness. Examples include, without limitation, myocardial infarction, heart failure, a cancer, such as breast cancer or other cancer, and pulmonary fibrosis. In some examples, a subject may receive additional treatments, in combination with administration of RNA molecules in accordance with the present disclosure. In some examples, an additional treatment may include a third RNA molecule. As a non-limiting illustrative example, a cancer patient, such as a subject having breast cancer, may be administered a first and second RNA molecule as disclosed herein, for promoting expression of a therapeutic compound in an on-target cell being a tumor cell. In a non-limiting example, a first RNA molecule may include a sequence encoding a translation suppressor protein (such as Cas6, L7Ae, or L30e) and include a miR recognition element for one or more of miR-155, miR-10b, miR-181a, and miR-181b. A second RNA molecule may include a sequence encoding a protein of interest (such as, e.g., a p53 protein, a Herpes Simplex Virus type 1 thymidine kinase, a deltex protein, an E1A protein, a cystatin SA protein, a cystatin E/M protein, a caspase 9 protein, or a type 2 phosphatidylinositol-5-phosphate 4-kinase gamma protein, and a recognition element for miR-143 and miR-122. For example, the first RNA molecule may include a recognition sequence for miR-155, miR-10b, miR-181a, and miR-181b and encode a Cas6 and a second RNA molecule may include a recognition sequence for miR-143 and miR-122 and encode a type 2 phosphatidylinositol-5-phosphate 4-kinase gamma protein. A cancer patient, such as a subject with breast cancer, may be administered a first and second RNA molecule in accordance with any of the foregoing example. Another example may further include administration of a cancer treatment in combination with the first and second RNA molecule (e.g., chemotherapeutic agent, anti-tumor antibody treatment, checkpoint inhibitor treatment (e.g., antibody or other treatment that inhibit or block CTLA4, PD-1, or PD-L1), radiation therapy, surgery, etc.). In an example, the cancer treatment in addition to the first and second RNA may be a third RNA, such as an RNA that encodes a cancer therapeutic (e.g., that encodes an antibody that inhibits CTLA4, PD-1, or PD-L1). In an example, such third RNA molecule may include on or more modRNA nucleotide. In an example, such RNA molecule may be a modRNA molecule encoding a CTLA4 antibody.

### EXAMPLES

The following examples are intended to illustrate particular embodiments of the present disclosure, but are by no means intended to limit the scope thereof.

### Example 1: General Methods

Cell culture—All animal procedures were performed under protocols approved by the Icahn School of Medicine at Mount Sinai Institutional Animal Care and Use Committee (IACUC). For in vitro experiments, cardiac cells were isolated from C57bl/6J P0-P4 neonate hearts (mice purchased from Charles River laboratories) using Pierce Primary Cardiomyocyte Isolation Kit. To obtain the mixture of cardiomyocytes and non-cardiomyocytes, the culture medium was changed at 24 hours instead of 4 hours (followed to obtain pure cardiomyocyte population). Hek293 cells were also used for in vitro transfection assays. Hek293 cells were maintained in DMEM culture medium+10% FBS and split every 48 hours. For luciferase modRNA transfections, cells were plated in 24-well plates at a density of 1×10⁵ cells/well and transfected at 24 hours.

Construction of IVT templates and synthesis of modRNA-ModRNAs were transcribed in vitro from plasmid templates (RNA transcriptions of examples of open reading frame sequences used to make modRNA are listed in Table 6). Using a customized ribonucleotide blend of anti-reverse cap analog, 3-O-Me-m7G(5′)ppp(5′)G (6 mM, TriLink Biotechnologies), guanosine triphosphate (1.5 mM, Life Technology), adenosine triphosphate (7.5 mM, Life Technology), cytidine triphosphate (7.5 mM, Life Technology) and N1-Methylpseudouridine-5′-Triphosphate (7.5 mM, TriLink Biotechnologies) as described previously (Sultana, N, Sharkar, MTK, Hadas, Y, Chepurko, E, and Zangi, L (2021). In Vitro Synthesis of Modified RNA for Cardiac Gene Therapy. Methods Mol Biol 2158: 281-294). mRNA was purified using the Megaclear kit (Life Technology) and treated with antarctic phosphatase (New England Biolabs), followed by re-purification using the Megaclear kit. mRNA was quantitated by Nanodrop (Thermo Scientific), precipitated with ethanol and ammonium acetate and resuspended in 10 mM TrisHCl, 1 mM EDTA.

modRNA transfection: In Vitro Transfection—2.5 ug Luc mRNA with or without Cas6 (at ratio of 4:1) was were transfected into Hek 293 or primary cardiac cells, containing CMs and non-CMs, using RNAiMAX transfection reagent (Life Technologies). The transfection mixture was prepared according to the manufacturer's protocol, and then it was added to cells cultured in basal medium containing growth factors and 2% fetal bovine serum (FBS) (Lonza). Then, 18 hr post-transfection, cells were processed for immunocytochemistry or bioluminescence imaging.

In Vivo Transfection—For intramyocardial injections, 100 ug of Luc, mCherry, Cre, AC or AC SMRTs 2.0 was delivered in total volume of 60 uL sucrose citrate buffer, as described in our previous publication paper(6) at the time of open chest surgery. For intravenous delivery, the modRNA was formulated with PCNP JetRNA (polyplus) reagent according to the manufacturer's recommendation and was concentrated using concentration filters (Millipore) to the approximate volume of 200 ul/mice. Mice were injected intravenously four times at the intervals of three days.

Immunostaining—Frozen heart sections were rehydrated in PBS for 5 min, followed by permeabilization in PBS with 0.1% triton x 100 (PBST) for 7 min. Further, the samples were blocked with blocking serum (5% Donkey normal serum in PBST) for 2 hrs at room temperature, and primary antibodies (see a complete list of primary antibodies used for this study in Table 4) diluted in blocking serum were added for overnight incubation at 4° C. Next day the slides were washed three times with PBST (5 min per wash), then incubated with a secondary antibody (Invitrogen, 1:200) diluted in PBST for 2 hours at room temperature. The samples were washed three times in PBST (5 min per wash) and stained with Hoechst 33342 (1 g/ml) diluted in PBST for 7 min. After five 4-min washes with PBST and one 4-min wash with tap water, slides were mounted with mounting medium (VECTASHIELD) for imaging. Stained slides were stored at 4° C.

To immunostain neonatal mouse cardiac cells, cells were fixed on coverslips with 4% PFA for 15 min at room temperature and then washed three times with PBS. Following permeabilization with 0.5% Triton X in PBS for 10 min at room temperature, cells were blocked with 5% normal goat/donkey serum+0.5% Tween 20 for 30 min. Coverslips were incubated with primary antibodies for 1 hr in a humid chamber at room temperature, followed by incubation with corresponding secondary antibodies conjugated to Alexa Fluor 488, Alexa Fluor 647, and Alexa Fluor 555, as well as Hoechst 33342 staining for nuclei visualization (all from Invitrogen). TUNEL staining was performed according to the kit manufacturer's instructions (In-Situ Cell Death Detection Kit, Fluorescein, Cat #11684795910, Roche). TUNEL quantification was then performed on the heart sections with ImageJ software. The fluorescent images were taken on a Zeiss fluorescent microscope. List of antibodies used for immunostaining are listed in supplemental Table 4.

IR surgery and injections—10-12 week old mice (males and females) were pre oxygenated with 100% oxygen for 5 min and anaesthetized with a combination of 10 mg/kg Alfaxalone (ALFAXAN®), 1 mg/kg Medetomidine (Medeson®) and 2 mg/kg Midazolam (DORMICUN®). After thoracic fur trimming and application of a gel solution to avoid corneal drying, animals were intubated until they lost the pedal reflex and laid in right lateral recumbency, and chlorhexidine 1% was applied for skin disinfection. Animal body temperature was maintained using a warmed 38° C. pad and was ventilated with 100% oxygen (120 breaths per minutes 8 ml/kg of tidal volume) using Minivent 680 ventilator (Harvard apparatus). To reach the left ventricle of the heart, a 0.5 cm skin incision was made over the projection of the 4th costal space. Without damaging left pectoral muscles, thoracic space was exposed between the 4th and 5th ribs. After a soft pericardiectomy, the left anterior descendent (LAD) coronary artery was visualized and occluded with a small piece of P10 fixed laterally with 7/0 nylon suture (PROLENE®). LAD coronary artery reperfusion was allowed after 60 minutes, just releasing the suture and tubing pressure over the vessel. Reperfusion was confirmed after visualization of reddish heart color. Ribs and skin incision were sutured closed by planes with 6/0 silk suture. For recovery, 2 mg/kg of atipamezol (REVERTOR®) was inoculated IP and mice were extubated once they become conscious. To keep post-surgery analgesia, mice were injected with 0.1 mg/kg of buprenorphine (BUPREX®) and 320 mg/kg of Paracetamol in drinking water for 3 days.

Detection of Luciferase Expression Using the IVIS System-Bioluminescence imaging of the transfected cells (18h) or I.V. injected mice was taken at different time points (day 1-day 7) in the IVIS system. To visualize cells expressing firefly luciferase invitro, D-luciferin was added to the cell-culture plate, and an image was taken in the IVIS system (IVIS Spectrum National Center for Research Resources [NCRR] S10-RR026561-01 at the Preclinical Small Imaging Core at Mount Sinai Medical Center). To visualize tissues expressing Luc in vivo, mice were anesthetized with isoflurane (Abbott Laboratories), and luciferin (150 mg/g body weight; Sigma) was injected intraperitoneally. Mice were imaged using an IVIS imaging system (IVIS Spectrum NCRR S10-RR026561-01 at the Preclinical Small Imaging Core at Mount Sinai Medical Center) every 2 min until the Luc signal reached a plateau. Imaging data were analyzed and quantified with Living Image software.

MRI and echo—CFW mice (8-10 weeks old) treated with Luc, AC or AC SMRTs 2.0 modRNAs, were subjected to MRI assessment on day 28 post I/R surgery. We obtained delayed-enhancement CINE images on a 7-T Bruker Pharmascan with cardiac and respiratory gating (SA Instruments). Mice were anesthetized with 1-2% isoflurane/air mixture. ECG, respiratory and temperature probes were placed on the mouse, which was kept warm during scans. Imaging was performed 10 to 20 min after IV injection of 0.3 mmol/kg gadolinium-diethylene triamine pentaacetic acid. A stack of 8 to 10 short-axis slices spanning from the heart apex to its base were acquired with an ECG triggered and respiratory-gated FLASH sequence with the following parameters: echo time (TE) 2.7 msec with resolution of 200 μm×200 μm; slice thickness of 1 mm; 16 frames per R-R interval; 4 excitations with flip angle at 60°. Ejection fraction was calculated as the difference between end-diastolic and end-systolic volumes, divided by the end-diastolic volume. MRI studies and analyses were performed blinded to treatment groups. For Echo evaluation of left ventricular systolic function, a visual sonics (Vevo 2100 Imaging) equipped with a 40 MHz mouse ultrasound probe was used. Fractional shortening was calculated based on end diastolic and end systolic dimensions obtained from M-mode ultrasound.

Masson's trichome staining—Masson's trichome staining was performed to evaluate scar size in the LV post IR and intracardiac or intravenous Luc, AC, AC SMRTs modRNA treatments. The OCT frozen transverse heart sections were air dried for 30 min to 1 hr at room temperature before proceeding to staining. Slides were pre-stained with Bouin's Solution for 45 mins at 55 C. Next, slides were kept in Weigert's Iron Hematoxylin, Biebrich Scarlet-Acid Fucshin, Phosphotungstic/Phosphomolybdic Acid Solution and Aniline Blue Solution for the times suggested by manufacturers. Thereafter, tissue samples were differentiated with acetic acid for 2 mins and dehydrated through 95% ethyl alcohol and absolute ethyl alcohol. After being cleared using xylene, slides were mounted with Permount mounting medium (Fisher Scientific). Images were collected using a bright field microscope and scar size analysis was conducted using ImageJ software.

CBC and serum liver enzymes—Female and male, ten-week-old CFW mice (Charles river laboratories) were injected with modRNA encapsulated with JetRNA at a dose of 100 ug and sacrificed at 24 h later or 4 weeks post I/R injury. Blood and blood serum was collected and analyzed for complete blood count and liver enzymes respectively by Mount Sinai pathology, New York.

H&E staining—H&E staining was performed according to standard protocol. The paraffin embedded heart, spleen, lung and liver sections were air dried for 30 min to 1 hr at room temperature, then hydrated in PBS for 10 mins. The slides were kept in Hematoxylin solution for 2 mins and washed with tap water for 5 mins. Thereafter, the sections were stained using eosin solution for 1 min and washed with tap water for 5 mins. The slides were transferred to PBS for 5 mins. Sections were then dehydrated in 100% ethanol and xylene for 1 min each. Finally, sections were mounted with Permount mounting medium (Fisher Scientific). The images were taken on a bright field microscope.

Cardiac Tissue Processing for Flow cytometry—Mice were sacrificed under deep anesthesia by 5% isoflurane. Mice were then perfused via the left ventricle with 10 mL of ice-cold PBS, and the infarcted regions were excised and weighed. Infarcted regions were minced and digested with 450 U ml-1 collagenase I, 125 U ml-1 collagenase XI, 60 U ml-1 DNase I and 60 U ml-1 hyaluronidase (Sigma) in PBS for 60 min at 37° C. with gentle agitation. Cells were then passed through a 100 m filter to obtain a single-cell suspension and washed with PBS for downstream applications.

Flow Cytometry—Single-cell suspensions were incubated for 5 minutes with 1× red blood cell (RBC) lysis buffer (BioLegend) to eliminate RBC contamination and washed in PBS. Resultant RBC-free single-cell suspensions were stained for 20 minutes at 4° C. in FACS buffer (0.5% BSA and 2 mM EDTA in PBS) with the following antibody cocktail at a concentration of 1:700 unless otherwise specified: BV711 anti-CD45.2 (BioLegend, clone: 30-F11), PerCP/Cyanine5.5 anti-CD11b (BioLegend, clone: M1/70), BV605 anti-Ly-6C (BioLegend, clone: HK1.4), PerCP-efluor710 anti-Ly-6G (Invitrogen, clone: 1A8-Ly6g), FITC anti-F4/80 (BioLegend, clone: BM8; 1:350 dilution), PE/Dazzle 594 anti-CD64 (BioLegend, clone: X54-5/7.1; 1:350 dilution), AlexaFluor 647 anti-I-A/I-E (MHC-II) (BioLegend, clone: M5/114.15.2), and BV650 anti-CD192 (CCR2) (BD Biosciences, clone: M5/114.15.2, 1:350 dilution). Following the incubation, cells were washed with PBS and cells were stained with Fixable Viability Dye efluor-506 (eBioscience, 1:1000 dilution) for 10 minutes at 4° C. in PBS. Finally, cells were washed with FACS buffer and resuspended in FACS buffer for acquisition. Samples were acquired on Aurora Cytometer (Cytek Biosciences) and analyzed using FlowJo software (Treestar).

Mouse Flow Cytometry Gating—Live (negative for viability dye) singlet cells were identified as 1) neutrophils (CD45.2+, CD11b+, Ly-6Cint and Ly-6G+); 2) Ly-6Chi monocytes (CD45.2+, CD11b+, Ly-6Chi and Ly-6G−); 3) Cardiac Macrophages (CD45.2+, CD11b+, Ly-6C−, Ly-6G−, F4/80+ and CD64+). Cardiac macrophages were then further subdivided as 4) inflammatory monocyte-derived macrophages (CD45.2+, CD11b+, Ly-6C−, Ly-6G−, F4/80+, CD64+, MHC-II+/− and CCR2+).

RNA isolation and gene expression profiling using Real-Time PCR.—Total RNA was isolated using the Quick-RNA Miniprep Kit and reverse transcribed using ISCRIPT™ cDNA Synthesis Kit (Biorad) according to the manufacturer's instructions. Real-time qPCR analyses were performed on a Mastercycler Realplex 4 Sequence Detector (Eppendorf) using PerfeCTa SYBR Green FastMix (QuantaBio). Data were normalized to GAPDH expression; fold-changes in gene expression were determined by the aaCT method and presented relative to an internal control. PCR primer sequences are listed in Table 5.

Statistical analysis—Statistical significance was determined by Unpaired two-tailed t-test, One-way ANOVA and Tukey's Multiple Comparison Test, as detailed in the respective Figure legends. P-value <0.05 was considered significant. All graphs represent average values, and values were reported as mean±standard error of the mean. P<0.0001; ***, P<0.001; **, P<0.01; *<, P<0.05; N. S, Not Significant.

TABLE 4
List of Immunostaining antibodies

| Antibody Name | Company | Catalog number |
|---|---|---|
| Alpha Actinin | Abcam | Ab9465 |
| Vimentin | Abcam | Ab92547 |
| tdTomato | Sicgen | AB8181-200 |
| cTNI | Abcam | Ab47003 |
| GFP | Abcam | Ab13970 |

TABLE 5
Primer Sequences for qPCR in this study

| Gene | Forward | Reverse |
|---|---|---|
| AC | ACAGGATTCAAACCAGGACTGT | TGGGCATCTTTCCTTCCGAA |
| | SEQ ID NO: 41 | SEQ ID NO: 42 |
| Sphk1 | ATACTCACCGAACGGAAGAACC | CCATTAGCCCATTCACCACCTC |
| | SEQ ID NO: 43 | SEQ ID NO: 44 |
| GAPDH | AGGTCGGTGTGAACGGATTTG | TGTAGACCATGTAGTTGAGGTCA |
| | SEQ ID NO: 45 | SEQ ID NO: 46 |

TABLE 6
Nucleotide sequences encoding non-limiting
examples of genes of interest.

| SEQ | | |
|---|---|---|
| ID | Gene of | |
| NO | interest | Sequence |
| 47 | Luc | AUGGCCGAUGCUAAGAACAUUAAGAAGGGCCCUGC |
| | | UCCCUUCUACCCUCUGGAGGAUGGCACCGCUGGCGA |
| | | GCAGCUGCACAAGGCCAUGAAGAGGUAUGCCCUGG |
| | | UGCCUGGCACCAUUGCCUUCACCGAUGCCCACAUUG |
| | | AGGUGGACAUCACCUAUGCCGAGUACUUCGAGAUG |
| | | UCUGUGCGCCUGGCCGAGGCCAUGAAGAGGUACGG |
| | | CCUGAACACCAACCACCGCAUCGUGGUGUGCUCUGA |
| | | GAACUCUCUGCAGUUCUUCAUGCCAGUGCUGGGCGC |
| | | CCUGUUCAUCGGAGUGGCCGUGGCCCCUGCUAACGA |
| | | CAUUUACAACGAGCGCGAGCUGCUGAACAGCAUGG |
| | | GCAUUUCUCAGCCUACCGUGGUGUUCGUGUCUAAG |
| | | AAGGGCCUGCAGAAGAUCCUGAACGUGCAGAAGAA |
| | | GCUGCCUAUCAUCCAGAAGAUCAUCAUCAUGGACUC |
| | | UAAGACCGACUACCAGGGCUUCCAGAGCAUGUACAC |
| | | AUUCGUGACAUCUCAUCUGCCUCCUGGCUUCAACGA |
| | | GUACGACUUCGUGCCAGAGUCUUUCGACAGGGACA |
| | | AAACCAUUGCCCUGAUCAUGAACAGCUCUGGGUCU |
| | | ACCGGCCUGCCUAAGGGCGUGGCCCUGCCUCAUCGC |
| | | ACCGCCUGUGUGCGCUUCUCUCACGCCCGCGACCCU |
| | | AUUUUCGGCAACCAGAUCAUCCCCGACACCGCUAUU |
| | | CUGAGCGUGGUGCCAUUCCACCACGGCUUCGGCAUG |
| | | UUCACCACCCUGGGCUACCUGAUUUGCGGCUUUCGG |
| | | GUGGUGCUGAUGUACCGCUUCGAGGAGGAGCUGUU |
| | | CCUGCGCAGCCUGCAAGACUACAAAAUUCAGUCUGC |
| | | CCUGCUGGUGCCAACCCUGUUCAGCUUCUUCGCUAA |
| | | GAGCACCCUGAUCGACAAGUACGACCUGUCUAACCU |
| | | GCACGAGAUUGCCUCUGGCGGCGCCCCACUGUCUAA |
| | | GGAGGUGGGCGAAGCCGUGGCCAAGCGCUUUCAUC |
| | | UGCCAGGCAUCCGCCAGGGCUACGGCCUGACCGAGA |
| | | CAACCAGCGCCAUUCUGAUUACCCCAGAGGGCGACG |
| | | ACAAGCCUGGCGCCGUGGGCAAGGUGGUGCCAUUC |
| | | UUCGAGGCCAAGGUGGUGGACCUGGACACCGGCAA |
| | | GACCCUGGGAGUGAACCAGCGCGGCGAGCUGUGUG |
| | | UGCGCGGCCCUAUGAUUAUGUCCGGCUACGUGAAU |
| | | AACCCUGAGGCCACAAACGCCCUGAUCGACAAGGAC |
| | | GGCUGGCUGCACUCUGGCGACAUUGCCUACUGGGAC |
| | | GAGGACGAGCACUUCUUCAUCGUGGACCGCCUGAA |
| | | GUCUCUGAUCAAGUACAAGGGCUACCAGGUGGCCCC |
| | | AGCCGAGCUGGAGUCUAUCCUGCUGCAGCACCCUAA |
| | | CAUUUUCGACGCCGGAGUGGCCGGCCUGCCCGACGA |
| | | CGAUGCCGGCGAGCUGCCUGCCGCCGUCGUCGUGCU |
| | | GGAACACGGCAAGACCAUGACCGAGAAGGAGAUCG |
| | | UGGACUAUGUGGCCAGCCAGGUGACAACCGCCAAG |
| | | AAGCUGCGCGGCGGAGUGGUGUUCGUGGACGAGGU |
| | | GCCCAAGGGCCUGACCGGCAAGCUGGACGCCCGCAA |
| | | GAUCCGCGAGAUCCUGAUCAAGGCUAAGAAAGGCG |
| | | GCAAGAUCGCCGUGUAA |
| 48 | AC | AUGCCGGGCCGGAGUUGCGUCGCCUUAGUCCUCCUG |
| | | GCUGCCGCCGUCAGCUGUGCCGUCGCGCAGCACGCG |
| | | CCGCCGUGGACAGAGGACUGCAGAAAAUCAACCUA |
| | | UCCUCCUUCAGGACCAACGUACAGAGGUGCAGUUCC |
| | | AUGGUACACCAUAAAUCUUGACUUACCACCCUACAA |
| | | AAGAUGGCAUGAAUUGAUGCUUGACAAGGCACCAG |
| | | UGCUAAAGGUUAUAGUGAAUUCUCUGAAGAAUAUG |
| | | AUAAAUACAUUCGUGCCAAGUGGAAAAAUUAUGCA |
| | | GGUGGUGGAUGAAAAAUUGCCUGGCCUACUUGGCA |
| | | ACUUUCCUGGCCCUUUUGAAGAGGAAAUGAAGGGU |
| | | AUUGCCGCUGUUACUGAUAUACCUUUAGGAGAGAU |
| | | UAUUUCAUUCAAUAUUUUUUAUGAAUUAUUUACCA |
| | | UUUGUACUUCAAUAGUAGCAGAAGACAAAAAAGGU |
| | | CAUCUAAUACAUGGGAGAAACAUGGAUUUUGGAGU |
| | | AUUUCUUGGGUGGAACAUAAAUAAUGAUACCUGGG |
| | | UCAUAACUGAGCAACUAAAACCUUUAACAGUGAAU |
| | | UUGGAUUUCCAAAGAAACAACAAAACUGUCUUCAA |
| | | GGCUUCAAGCUUUGCUGGCUAUGUGGGCAUGUUAA |
| | | CAGGAUUCAAACCAGGACUGUUCAGUCUUACACUG |
| | | AAUGAACGUUUCAGUAUAAAUGGUGGUUAUCUGGG |
| | | UAUUCUAGAAUGGAUUCUGGGAAAGAAAGAUGUCA |
| | | UGUGGAUAGGGUUCCUCACUAGAACAGUUCUGGAA |
| | | AAUAGCACAAGUUAUGAAGAAGCCAAGAAUUUAUU |
| | | GACCAAGACCAAGAUAUUGGCCCCAGCCUACUUUAU |
| | | CCUGGGAGGCAACCAGUCUGGGGAAGGUUGUGUGA |
| | | UUACACGAGACAGAAAGGAAUCAUUGGAUGUAUAU |
| | | GAACUCGAUGCUAAGCAGGGUAGAUGGUAUGUGGU |
| | | ACAAACAAAUUAUGACCGUUGGAAACAUCCCUUCU |
| | | UCCUUGAUGAUCGCAGAACGCCUGCAAAGAUGUGU |
| | | CUGAACCGCACCAGCCAAGAGAAUAUCUCAUUUGA |
| | | AACCAUGUAUGAUGUCCUGUCAACAAAACCUGUCC |
| | | UCAACAAGCUGACCGUAUACACAACCUUGAUAGAU |
| | | GUUACCAAAGGUCAAUUCGAAACUUACCUGCGGGA |
| | | CUGCCCUGACCCUUGUAUAGGUUGGUGA |
| 49 | Cre | AUGUCCAAUUUACUGACCGUACACCAAAAUUUGCC |
| | | UGCAUUACCGGUCGAUGCAACGAGUGAUGAGGUUC |
| | | GCAAGAACCUGAUGGACAUGUUCAGGGAUCGCCAG |
| | | GCGUUUUCUGAGCAUACCUGGAAAAUGCUUCUGUC |
| | | CGUUUGCCGGUCGUGGGCGGCAUGGUGCAAGUUGA |
| | | AUAACCGGAAAUGGUUUCCCGCAGAACCUGAAGAU |
| | | GUUCGCGAUUAUCUUCUAUAUCUUCAGGCGCGCGG |
| | | UCUGGCAGUAAAAACUAUCCAGCAACAUUUGGGCC |
| | | AGCUAAACAUGCUUCAUCGUCGGUCCGGGCUGCCAC |
| | | GACCAAGUGACAGCAAUGCUGUUUCACUGGUUAUG |
| | | CGGCGGAUCCGAAAAGAAAACGUUGAUGCCGGUGA |
| | | ACGUGCAAAACAGGCUCUAGCGUUCGAACGCACUG |
| | | AUUUCGACCAGGUUCGUUCACUCAUGGAAAAUAGC |
| | | GAUCGCUGCCAGGAUAUACGUAAUCUGGCAUUUCU |
| | | GGGGAUUGCUUAUAACACCCUGUUACGUAUAGCCG |
| | | AAAUUGCCAGGAUCAGGGUUAAAGAUAUCUCACGU |
| | | ACUGACGGUGGGAGAAUGUUAAUCCAUAUUGGCAG |
| | | AACGAAAACGCUGGUUAGCACCGCAGGUGUAGAGA |
| | | AGGCACUUAGCCUGGGGGUAACUAAACUGGUCGAG |
| | | CGAUGGAUUUCCGUCUCUGGUGUAGCUGAUGAUCC |
| | | GAAUAACUACCUGUUUUGCCGGGUCAGAAAAAAUG |
| | | GUGUUGCCGCGCCAUCUGCCACCAGCCAGCUAUCAA |
| | | CUCGCGCCCUGGAAGGGAUUUUUGAAGCAACUCAU |
| | | CGAUUGAUUUACGGCGCUAAGGAUGACUCUGGUCA |
| | | GAGAUACCUGGCCUGGUCUGGACACAGUGCCCGUG |
| | | UCGGAGCCGCGCGAGAUAUGGCCCGCGCUGGAGUU |
| | | UCAAUACCGGAGAUCAUGCAAGCUGGUGGCUGGAC |
| | | CAAUGUAAAUAUUGUCAUGAACUAUAUCCGUAACC |
| | | UGGAUAGUGAAACAGGGGCAAUGGUGCGCCUGCUA |
| | | GAAGAUGGCGAUUAG |
| 50 | Cas6 | AUGGACCACUACCUCGACAUUCGCUUGCGACCGGAC |
| | | CCGGAAUUUCCCCCGGCGCAACUCAUGAGCGUGCUC |
| | | UUCGGCAAGCUCCACCAGGCCCUGGUGGCACAGGGC |
| | | GGGGACAGGAUCGGCGUGAGCUUCCCCGACCUCGAC |
| | | GAAAGCCGCUCCCGGCUGGGCGAGCGCCUGCGCAUU |
| | | CAUGCCUCGGCGGACGACCUUCGUGCCCUGCUCGCC |
| | | CGGCCCUGGCUGGAAGGGUUGCGGGACCAUCUGCA |
| | | AUUCGGAGAACCGGCAGUCGUGCCUCACCCCACACC |
| | | GUACCGUCAGGUCAGUCGGGUUCAGGCGAAAAGCA |
| | | AUCCGGAACGCCUGCGGCGGCGGCUCAUGCGCCGGC |
| | | ACGAUCUGAGUGAGGAGGAGGCUCGGAAACGCAUU |
| | | CCCGAUACGGUCGCGAGAGCCUUGGACCUGCCCUUC |
| | | GUCACGCUACGCAGCCAGAGCACCGGACAGCACUUC |
| | | CGUCUCUUCAUCCGCCACGGGCCGUUGCAGGUGACG |
| | | GCAGAGGAAGGAGGAUUCACCUGUUACGGGUUGAG |
| | | CAAAGGAGGUUUCGUUCCCUGGUUCUGA |
| 51 | nmCherry | AUGGUGAGCAAGGGCGAGGAGGAUAACAUGGCCAU |
| | | CAUCAAGGAGUUCAUGCGCUUCAAGGUGCACAUGG |
| | | AGGGCUCCGUGAACGGCCACGAGUUCGAGAUCGAG |
| | | GGCGAGGGCGAGGGCCGCCCCUACGAGGGCACCCAG |
| | | ACCGCCAAGCUGAAGGUGACCAAGGGUGGCCCCCUG |
| | | CCCUUCGCCUGGGACAUCCUGUCCCCUCAGUUCAUG |
| | | UACGGCUCCAAGGCCUACGUGAAGCACCCCGCCGAC |
| | | AUCCCCGACUACUUGAAGCUGUCCUUCCCCGAGGGC |
| | | UUCAAGUGGGAGCGCGUGAUGAACUUCGAGGACGG |
| | | CGGCGUGGUGACCGUGACCCAGGACUCCUCCCUGCA |
| | | GGACGGCGAGUUCAUCUACAAGGUGAAGCUGCGCG |
| | | GCACCAACUUCCCCUCCGACGGCCCCGUAAUGCAGA |
| | | AGAAGACCAUGGGCUGGGAGGCCUCCUCCGAGCGG |
| | | AUGUACCCCGAGGACGGCGCCCUGAAGGGCGAGAUC |
| | | AAGCAGAGGCUGAAGCUGAAGGACGGCGGCCACUA |
| | | CGACGCUGAGGUCAAGACCACCUACAAGGCCAAGAA |
| | | GCCCGUGCAGCUGCCCGGCGCCUACAACGUCAACAU |
| | | CAAGUUGGACAUCACCUCCCACAACGAGGACUACAC |
| | | CAUCGUGGAACAGUACGAACGCGCCGAGGGCCGCCA |
| | | CUCCACCGGCGGCAUGGACGAGCUGUACAAGAAGCG |
| | | GCCCGCAGCAACCAAGAAGGCAGGACAGGCCAAGAA |
| | | GAAGAAGUAAG |
| 52 | nGFP | AUGGUGAGCAAGGGCGAGGAGCUGUUCACCGGGGU |
| | | GGUGCCCAUCCUGGUCGAGCUGGACGGCGACGUAA |
| | | ACGGCCACAAGUUCAGCGUGUCCGGCGAGGGCGAG |
| | | GGCGAUGCCACCUACGGCAAGCUGACCCUGAAGUUC |
| | | AUCUGCACCACCGGCAAGCUGCCCGUGCCCUGGCCC |
| | | ACCCUCGUGACCACCCUGACCUACGGCGUGCAGUGC |
| | | UUCAGCCGCUACCCCGACCACAUGAAGCAGCACGAC |
| | | UUCUUCAAGUCCGCCAUGCCCGAAGGCUACGUCCAG |
| | | GAGCGCACCAUCUUCUUCAAGGACGACGGCAACUAC |
| | | AAGACCCGCGCCGAGGUGAAGUUCGAGGGCGACACC |
| | | CUGGUGAACCGCAUCGAGCUGAAGGGCAUCGACUU |
| | | CAAGGAGGACGGCAACAUCCUGGGGCACAAGCUGG |
| | | AGUACAACUACAACAGCCACAACGUCUAUAUCAUG |
| | | GCCGACAAGCAGAAGAACGGCAUCAAGGUGAACUU |
| | | CAAGAUCCGCCACAACAUCGAGGACGGCAGCGUGCA |
| | | GCUCGCCGACCACUACCAGCAGAACACCCCCAUCGG |
| | | CGACGGCCCCGUGCUGCUGCCCGACAACCACUACCU |
| | | GAGCACCCAGUCCGCCCUGAGCAAAGACCCCAACGA |
| | | GAAGCGCGAUCACAUGGUCCUGCUGGAGUUCGUGA |
| | | CCGCCGCCGGGAUCACUCUCGGCAUGGACGAGCUGU |
| | | ACAAGGGAGAUCCAAAAAAGAAGAGAAAGGUAGGC |
| | | GAUCCAAAAAAGAAGAGAAAGGUAGGUGAUCCAAA |
| | | AAAGAAGAGAAAGGUAUAA |
| 53 | anti-CTLA4 | AUGGAGUUUGGUCUUUCCUGGGUAUUCCUGGUGGC |
| | antibody | CCUUUUGCGCGGCGUACAAUGUGAAGCAAAGCUCC |
| | (9D9) | AAGAGAGUGGUCCCGUAUUGGUGAAACCUGGAGCA |
| | heavy | UCUGUCAAGAUGAGUUGUAAAGCCAGCGGAUACAC |
| | chain | UUUCACCGAUUAUUAUAUGAACUGGGUUAAACAAA |
| | | GCCACGGCAAAAGUCUGGAAUGGAUAGGGGUAAUC |
| | | AAUCCAUAUAACGGCGAUACCUCCUACAACCAAAAA |
| | | UUCAAGGGUAAGGCUACUUUGACAGUUGAUAAGUC |
| | | CUCUUCCACCGCAUACAUGGAGCUCAACUCACUGAC |
| | | UUCAGAAGACAGUGCUGUCUACUAUUGUGCUAGGU |
| | | AUUACGGAUCAUGGUUUGCUUACUGGGGGCAGGGU |
| | | ACUUUGAUCACCGUUAGUGCUGCCAAAACUACAGCC |
| | | CCCUCCGUGUACCCUCUCGCACCCGUUUGCGGUGAC |
| | | ACAACUGGCUCCUCUGUAACCCUGGGCUGCCUGGUA |
| | | AAGGGCUAUUUCCCAGAGCCCGUCACUCUGACUUGG |
| | | AACUCUGGGUCUCUGAGUUCAGGUGUACACACCUU |
| | | UCCUGCCGUACUUCAAAGCGAUCUCUACACACUUAG |
| | | CUCUAGUGUAACAGUAACAUCCUCUACCUGGCCAUC |
| | | ACAGUCUAUCACAUGCAACGUAGCACACCCUGCCUC |
| | | UAGCACAAAGGUUGACAAGAAAAUAGAGCCCAGAG |
| | | GACCAACCAUCAAGCCUUGUCCCCCAUGCAAAUGUC |
| | | CUGCCCCCAAUCUUUUGGGCGGUCCCUCCGUCUUCA |
| | | UCUUCCCCCCUAAAAUUAAGGACGUUCUCAUGAUU |
| | | UCCCUGUCUCCAAUCGUCACCUGCGUCGUAGUGGAU |
| | | GUUUCUGAAGACGACCCAGAUGUGCAAAUAAGUUG |
| | | GUUCGUCAAUAAUGUCGAGGUGCAUACAGCCCAAA |
| | | CUCAAACACAUAGAGAGGACUACAAUUCAACCCUU |
| | | AGGGUUGUUAGCGCACUUCCCAUACAGCACCAGGAC |
| | | UGGAUGAGUGGUAAAGAAUUCAAGUGCAAAGUGAA |
| | | CAAUAAGGACCUUCCCGCUCCUAUUGAGCGCACAAU |
| | | CAGUAAGCCUAAAGGUUCUGUCCGGGCACCUCAAG |
| | | UUUACGUCCUCCCACCCCCAGAGGAAGAGAUGACCA |
| | | AAAAGCAAGUGACUCUGACUUGUAUGGUAACAGAC |
| | | UUUAUGCCCGAGGAUAUUUAUGUUGAAUGGACUAA |
| | | CAACGGAAAGACUGAGCUGAACUAUAAGAAUACAG |
| | | AACCCGUCUUGGACUCAGACGGGAGUUAUUUCAUG |
| | | UAUUCCAAAUUGAGAGUAGAAAAAAAAAAUUGGGU |
| | | AGAGCGCAACUCUUAUAGUUGCAGUGUUGUACAUG |
| | | AAGGACUUCACAAUCACCAUACCACAAAGAGUUUU |
| | | UCUCGGACACCAGGGAAAUAG |
| 54 | anti-CTLA4 | AUGCUUCCCUCUCAGUUGAUCGGCUUCCUUCUGCUU |
| | antibody | UGGGUUCCCGCAAGCCGCGGUGACAUUGUGAUGAC |
| | (9D9) | CCAGACUACACUGUCUCUCCCUGUAUCCUUGGGGGA |
| | light | CCAAGCCUCCAUUAGCUGUAGAUCUUCACAAUCCAU |
| | chain | UGUCCAUUCCAAUGGGAAUACAUAUCUGGAGUGGU |
| | | AUUUGCAAAAGCCUGGUCAGUCUCCCAAACUGCUG |
| | | AUAUAUAAAGUGUCUAACCGAUUCAGUGGGGUGCC |
| | | AGAUAGGUUUAGCGGGAGCGGUUCAGGAACUGACU |
| | | UUACACUUAAGAUUUCAAGAGUUGAAGCCGAGGAU |
| | | CUUGGUGUUUACUAUUGCUUUCAGGGAUCUCAUGU |
| | | GCCAUACACUUUUGGAGGGGGCACUAAACUCGAGA |
| | | UUAAAAGGGCAGACGCUGCCCCUACAGUUAGUAUA |
| | | UUCCCACCAUCAAGCGAACAGUUGACUUCAGGAGG |
| | | GGCAUCCGUAGUAUGUUUUCUUAACAAUUUUUACC |
| | | CAAAAGACAUUAACGUCAAGUGGAAGAUCGAUGGG |
| | | UCAGAACGACAGAAUGGAGUUUUGAAUAGUUGGAC |
| | | UGACCAAGACUCCAAAGAUUCCACUUACUCCAUGUC |
| | | CUCAACCCUGACCUUGACCAAGGAUGAGUACGAAA |
| | | GACACAACUCCUAUACCUGCGAAGCCACUCACAAGA |
| | | CAAGCACCAGCCCCAUAGUUAAGUCUUUUAAUCGG |
| | | AACGAGUGCUAG |
| 55 | Pip4k2c | AUGGCGUCCUCCUCCGUCCCUCCCGCCACCGCACCC |
| | | GCGGCAGCUGGAGGCCCCGGCCCGGGAUUCGGCUUC |
| | | GCCUCCAAAACCAAGAAGAAGCAUUUCGUACAGCA |
| | | GAAAGUGAAGGUGUUCCGGGCCGCGGACCCGCUGG |
| | | UGGGCGUGUUCCUGUGGGGCGUCGCUCACUCGAUC |
| | | AAUGAGCUCAGCCAGGUACCGCCCCCAGUGAUGUUG |
| | | CUGCCAGAUGACUUUAAAGCCAGCUCCAAGAUCAA |
| | | GGUCAACAAUCACUUUUUCCAUAGAGAAAAUCUUC |
| | | CCAGUCAUUUCAAGUUCAAGGAGUAUUGUCCCCAG |
| | | GUCUUCAGGAACCUGAGAGAUCGGUUUGCCAUCGA |
| | | UGAUCACGAUUACUUGGUGUCCCUUACUCGAAGCCC |
| | | CCCAAGCGAAACUGAAGGCAGUGAUGGCCGUUUCC |
| | | UUAUCUCCUAUGACCGCACUCUGGUCAUCAAGGAA |
| | | GUGUCGAGCGAAGAUAUUGCGGAUAUGCACAGCAA |
| | | CCUCUCCAACUACCACCAGUACAUAGUGAAAUGUCA |
| | | UGGCAACACGCUUCUGCCCCAGUUCCUGGGCAUGUA |
| | | CCGAGUCAGUGUAGAAAAUGAAGACAGCUACAUGC |
| | | UCGUGAUGCGCAAUAUGUUUAGUCAUCGUCUUCCU |
| | | GUGCAUAGGAAGUAUGACCUCAAGGGCUCUCUAGU |
| | | GUCCCGGGAAGCCAGCGAUAAGGAAAAGGUUAAAG |
| | | AACUGCCAACACUAAAGGAUAUGGACUUUCUUAAC |
| | | AAGAACCAGAAAGUGUAUAUUGGUGAAGAAGAAAA |
| | | GAAAGUAUUCCUGGAGAAGCUGAAGCGAGAUGUGG |
| | | AGUUUCUAGUGCAGCUGAAGAUCAUGGACUACAGC |
| | | CUCCUGUUGGGCAUCCACGACAUCAUCCGGGGCUCU |
| | | GAACCCGAGGAAGAGGGGCCUGUGAGGGAGGAGGA |
| | | GUCUGAGUGGGAUGGGGACUGUAACCUGGCUGGAC |
| | | CUCCCGCCCUGGUGGGCUCCUAUGGUACCUCCCCUG |
| | | AGGGUAUCGGAGGCUACAUCCAUUCCCACCGGCCAC |
| | | UGGGCCCAGGAGAGUUUGAGUCCUUCAUCGAUGUC |
| | | UAUGCUAUCCGGAGUGCGGAGGGGGCCCCCCAGAA |
| | | GGAGGUGUAUUUCAUGGGCCUCAUUGACAUUCUGA |
| | | CACAGUAUGAUGCCAAGAAGAAAGCAGCUCAUGCA |
| | | GCCAAGACUGUCAAGCACGGGGCGGGGGCAGAGAU |
| | | CUCCACUGUCCAUCCUGAGCAGUAUGCUAAGCGAUU |
| | | CCUGGACUUUAUUGCCAACAUCUUUGCCUAA |
| 56 | p53 | AUGGAGGAGCCGCAGUCAGAUCCUAGCGUCGAGCCC |
| | | CCUCUGAGUCAGGAAACAUUUUCAGACCUAUGGAA |
| | | ACUACUUCCUGAAAACAACGUUCUGUCCCCCUUGCC |
| | | GUCCCAAGCAAUGGAUGAUUUGAUGCUGUCCCCGG |
| | | ACGAUAUUGAACAAUGGUUCACUGAAGACCCAGGU |
| | | CCAGAUGAAGCUCCCAGAAUGCCAGAGGCUGCUCCC |
| | | CCCGUGGCCCCUGCACCAGCAGCUCCUACACCGGCG |
| | | GCCCCUGCACCAGCCCCCUCCUGGCCCCUGUCAUCU |
| | | UCUGUCCCUUCCCAGAAAACCUACCAGGGCAGCUAC |
| | | GGUUUCCGUCUGGGCUUCUUGCAUUCUGGGACAGC |
| | | CAAGUCUGUGACUUGCACGUACUCCCCUGCCCUCAA |
| | | CAAGAUGUUUUGCCAACUGGCCAAGACCUGCCCUGU |
| | | GCAGCUGUGGGUUGAUUCCACACCCCCGCCCGGCAC |
| | | CCGCGUCCGCGCCAUGGCCAUCUACAAGCAGUCACA |
| | | GCACAUGACGGAGGUUGUGAGGCGCUGCCCCCACCA |
| | | UGAGCGCUGCUCAGAUAGCGAUGGUCUGGCCCCUCC |
| | | UCAGCAUCUUAUCCGAGUGGAAGGAAAUUUGCGUG |
| | | UGGAGUAUUUGGAUGACAGAAACACUUUUCGACAU |
| | | AGUGUGGUGGUGCCCUAUGAGCCGCCUGAGGUUGG |
| | | CUCUGACUGUACCACCAUCCACUACAACUACAUGUG |
| | | UAACAGUUCCUGCAUGGGCGGCAUGAACCGGAGGC |
| | | CCAUCCUCACCAUCAUCACACUGGAAGACUCCAGUG |
| | | GUAAUCUACUGGGACGGAACAGCUUUGAGGUGCGU |
| | | GUUUGUGCCUGUCCUGGGAGAGACCGGCGCACAGA |
| | | GGAAGAGAAUCUCCGCAAGAAAGGGGAGCCUCACC |
| | | ACGAGCUGCCCCCAGGGAGCACUAAGCGAGCACUGC |
| | | CCAACAACACCAGCUCCUCUCCCCAGCCAAAGAAGA |
| | | AACCACUGGAUGGAGAAUAUUUCACCCUUCAGAUC |
| | | CGUGGGCGUGAGCGCUUCGAGAUGUUCCGAGAGCU |
| | | GAAUGAGGCCUUGGAACUCAAGGAUGCCCAGGCUG |
| | | GGAAGGAGCCAGGGGGGAGCAGGGCUCACUCCAGC |
| | | CACCUGAAGUCCAAAAAGGGUCAGUCUACCUCCCGC |
| | | CAUAAAAAACUCAUGUUCAAGACAGAAGGGCCUGA |
| | | CUCAGACUGA |

In a non-limiting example, a recombinant RNA molecule as disclosed herein may include a nucleotide sequence that encodes for a gene of interest such as set out in Table 6, though the nucleotide sequence therefor may differ from the corresponding sequence as set out in Table 6 owing to, for example, codon redundancy. As skilled persons would also appreciate, an amino acid sequence of a gene of interest may vary from a sequence encoded by a nucleotide sequence of Table 6, such as an isoform of, for example, a luciferase (Luc), an acid ceramidase (AC), a Cre, a Cas6, an nmCherry, an nGFP, an anti-CTLA4 antibody (9D9) heavy chain, and anti-CTLA4 antibody (9D9) light chain, a Pip4k2c or a p53. For example, an amino acid sequence of a protein product of a gene of interest encoded by a recombinant RNA molecule in accordance with the present disclosure may be less than 100% homologous to an amino acid sequence of a protein product of a gene of interest encoded by a nucleotide sequence of Table 6. For example, an amino acid sequence of a protein product of a gene of interest encoded by a recombinant RNA molecule in accordance with the present disclosure may be 99% or more, or 97% or more, or 95% or more, or 92% or more, or 90% or more, or 87% or more, or 85% or more, or 80% or more, or 75% or more, or 75% or more homologous to an amino acid sequence of a protein product of a gene of interest encoded by a nucleotide sequence of Table 6.

### Example 2: CM-SMRTs 2.0 Structure, Organ and Cell Specificity and Pharmacokinetics Post Minimal Invasive Delivery (FEGs. 2A-2M)

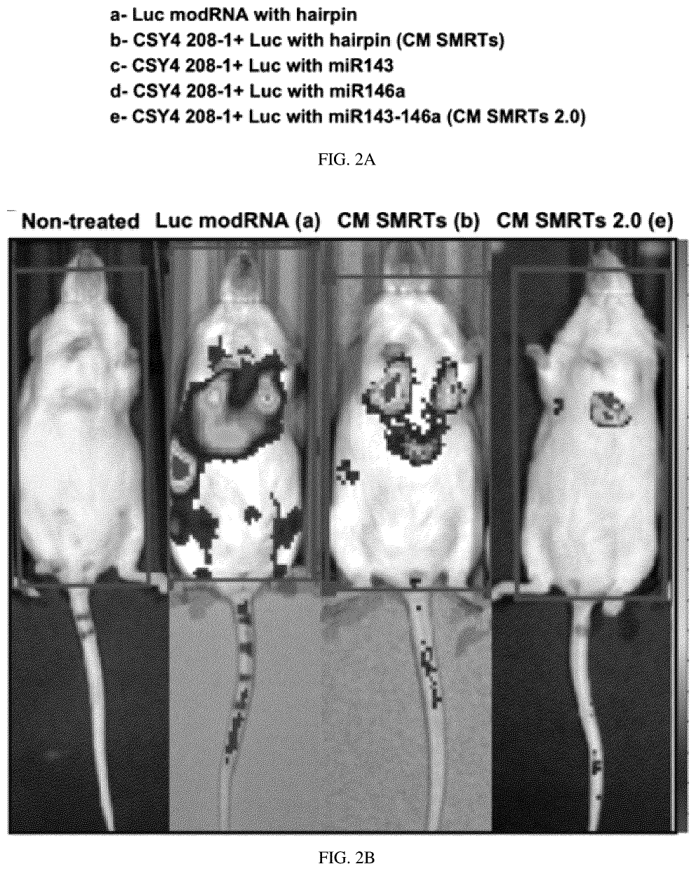

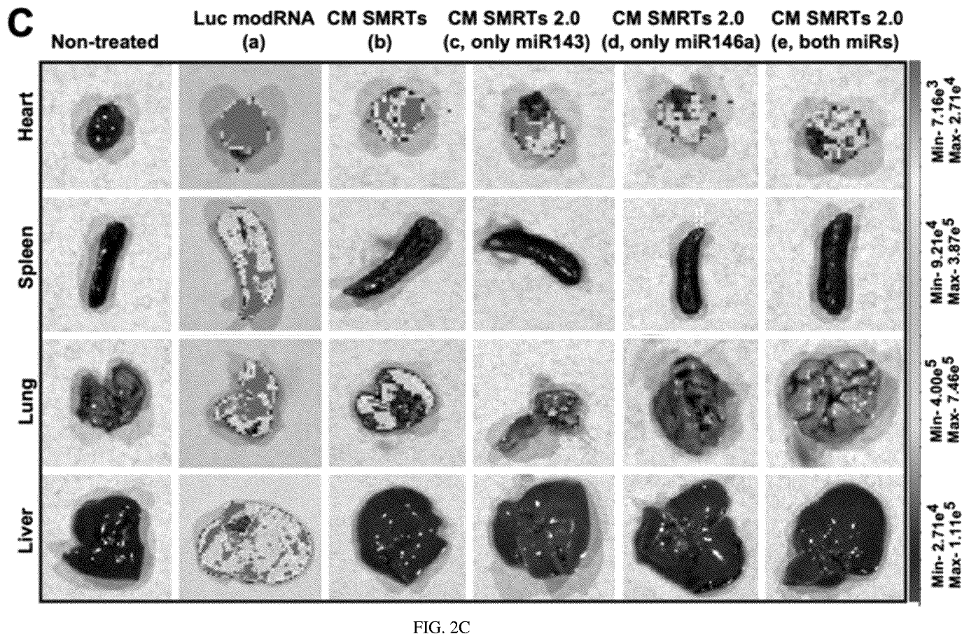

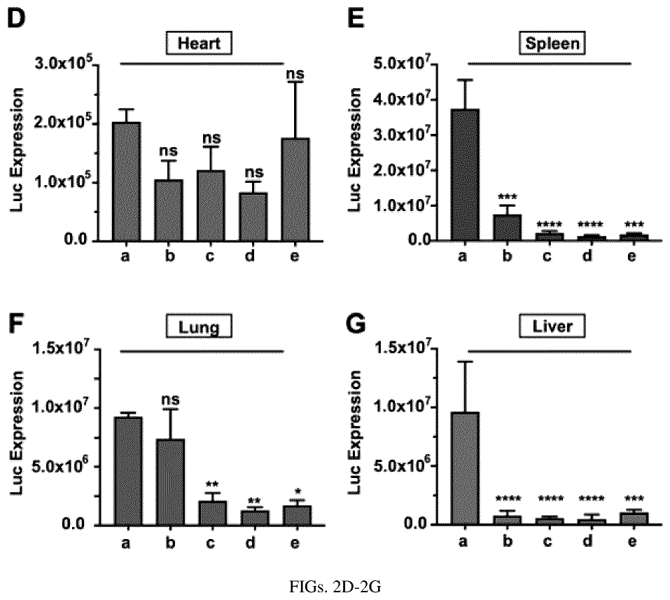

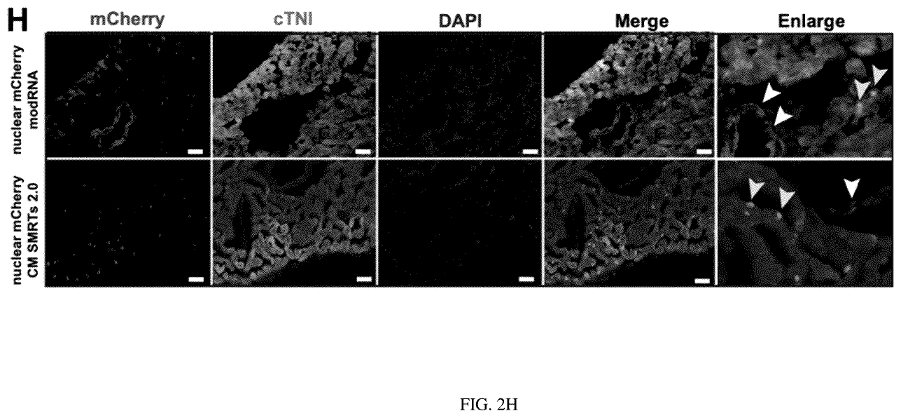

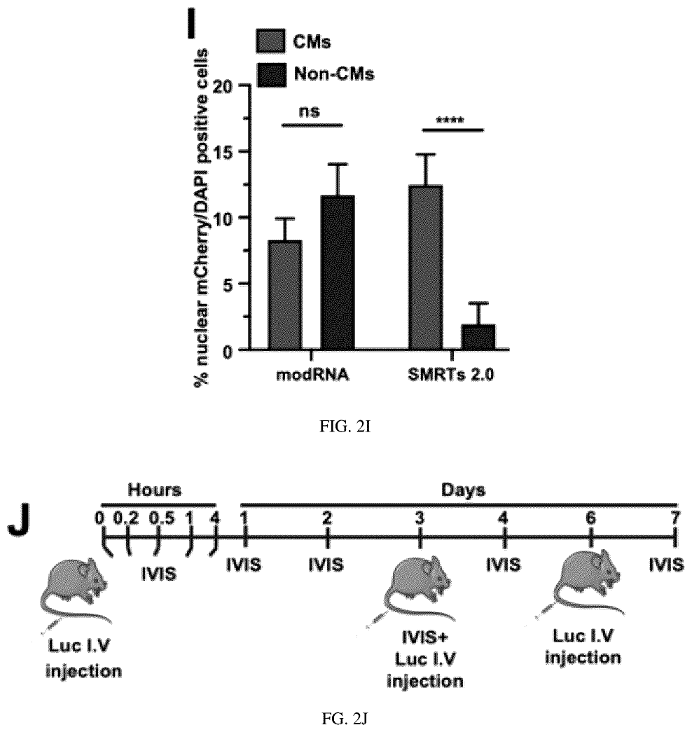

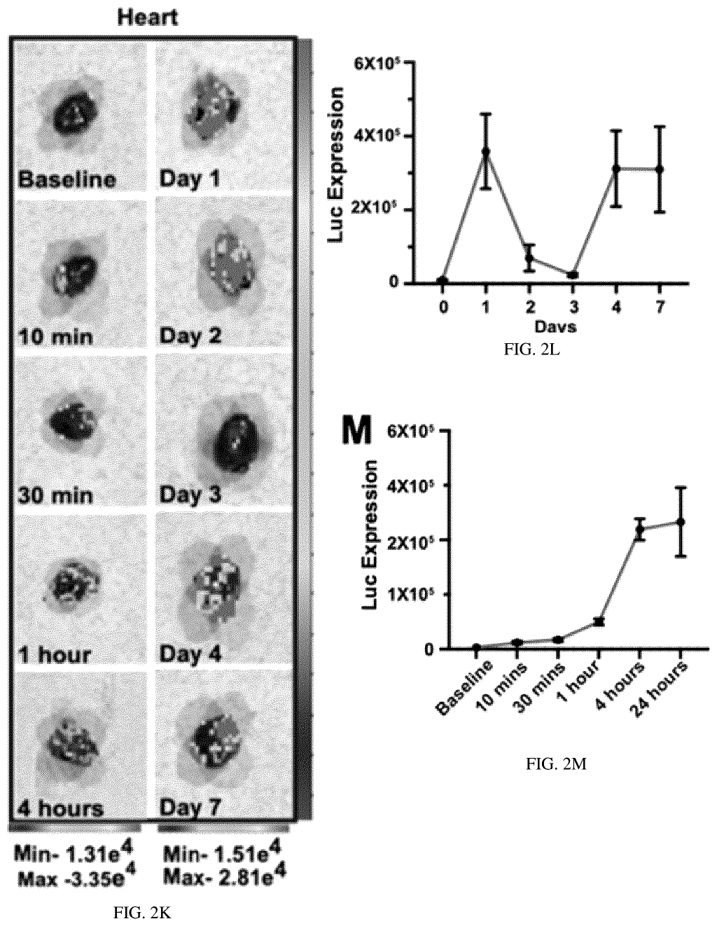

FIG. 2A shows treatments of groups of mice administered the following modified RNA (modRNA) treatments by tail vein injection: (a) encoding luciferase (Luc) or (mCherry); (b) non-limiting example of a cardiomyocyte (CM) modified mRNA translational system expression regulatory system wherein the first RNA molecule includes a miR recognition sequence (SMRT) in accordance with aspects of the present disclosure; (c), (d), (e) non-limiting examples of CM modRNA translational system expression regulatory system wherein the first and second RNA molecules includes a miR recognition sequence (SMRT 2.0), in accordance with aspects of the present disclosure.

FIGS. 2B and 2C show in vivo bioluminescence images for mice (B) treated with an example of a Luc modRNA, of a CM SMRT, and of a CM SMRT 2.0 and (C) for all major organs comparing expression between Luc modRNA, CM SMRT, Luc with miR143 or miR146a and CM SMRT 2.0 in CFW mice 24 hours post intravenous injection (IV).

FIGS. 2D-2G show relative luciferase expression in heart (D), spleen (E), lung (F), and liver (G), respectively, evaluated by bioluminescence imaging.

FIGS. 2H and 2I show nuclear mCherry expression in CMs and non-CMs post I.V injected with nuclear mCherry modRNA or with nuclear mCherry CM SMRT 2.0. (H) Hearts sectioned were stained for mCherry (left) and cardiomyocyte marker cTNI (second from left). FIG. 2I is a graph showing quantification of the example in H.

FIGS. 2J-2M show expression following a timeline for intravenously injecting an example of a Luc modRNA for the course of 7 days (J); heart in vivo bioluminescence images comparing Luc expression at different time points post I.V. Luc modRNA injection (K); short term (L) or long term (M) quantification of K. One-way ANOVA, Tukey's Multiple Comparison Test for C-F. Multiple unpaired t-test for h. Scale bar for H=25 μm.

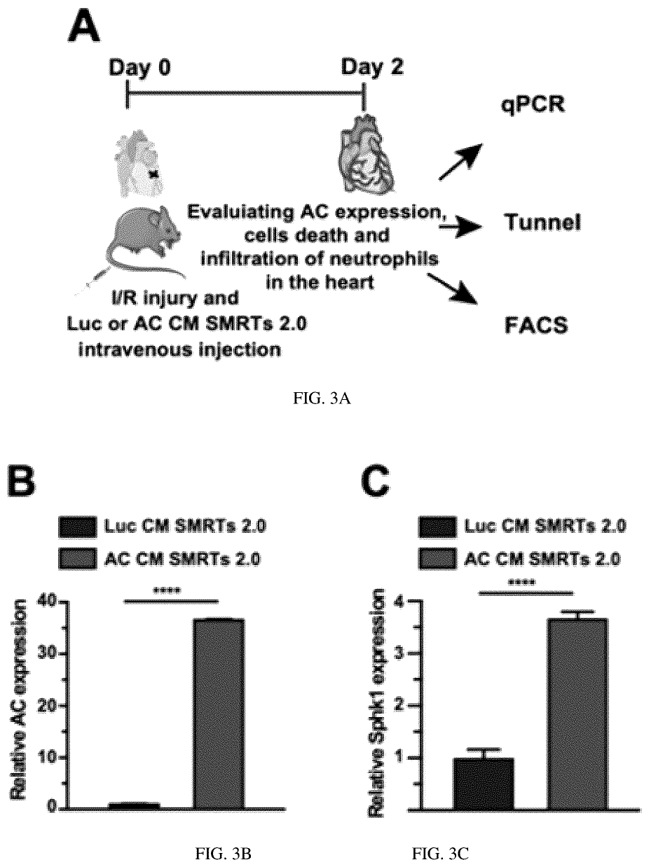

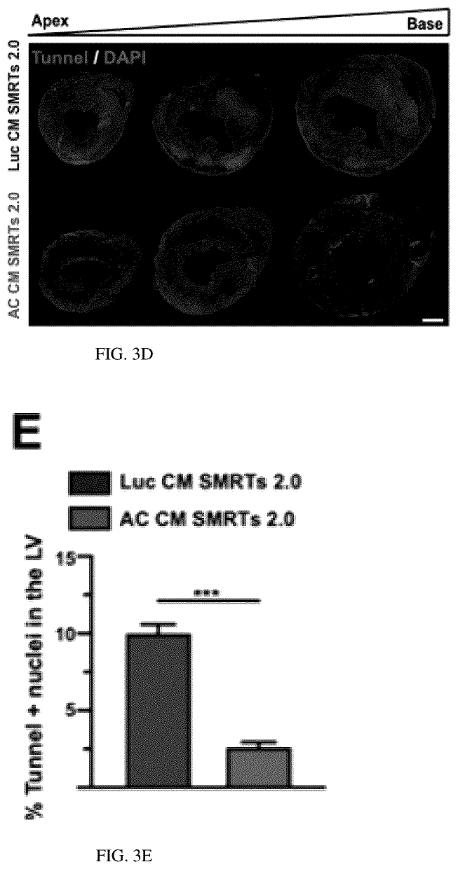

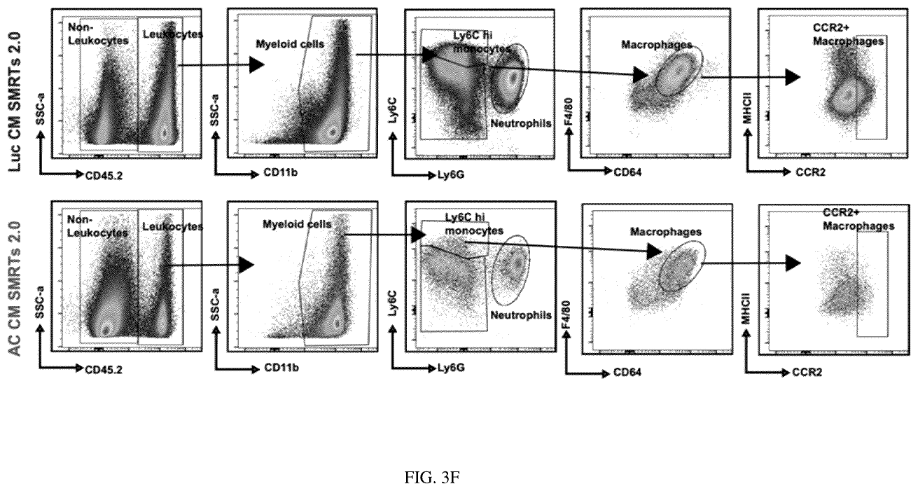

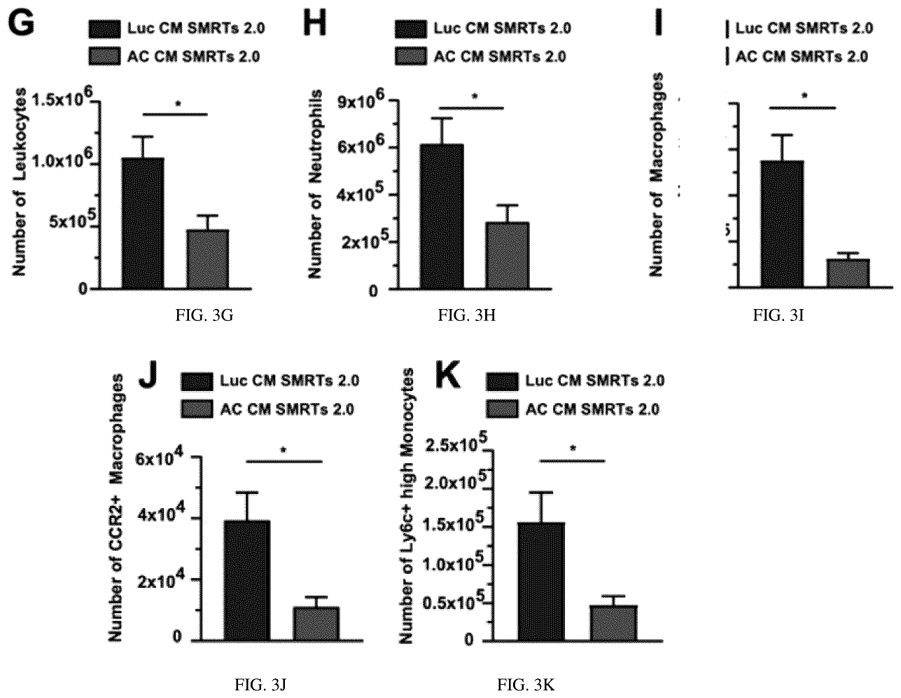

### Example 3: Attenuation of Cell Death and Inflammatory Response Post Minimal Invasive Delivery of AC CM-SMRTs 2.0 in an Ischaemia-Reperfusion (I/R) Cardiac Injury Model (FIGS. 3A-3K)

FIG. 3A shows an example of a timeline to evaluate AC expression in the heart, cell death and inflammatory response 2 days post I.V delivery of an example of Luc CM SMRT 2.0 (control) or of AC CM SMRT 2.0 in I/R cardiac injury model. B and C. qPCR for AC (B) and Sphkl (C) expression in the heart. D. Tunnel evaluation of cell death (red staining) 3 days post I/.R. E. quantification of the experiment in D. (F) FACS analysis to evaluate inflammatory response 2 days post I/R. G-H. number of Leukocytes (G), Neutrophils (H), Macrophages (I), CCR2+ Macrophages (J) and LY6c+ high Monocytes (K) in heart treated with an example of a Luc or of an AC CM SMRT 2.0 2 days post I/R.

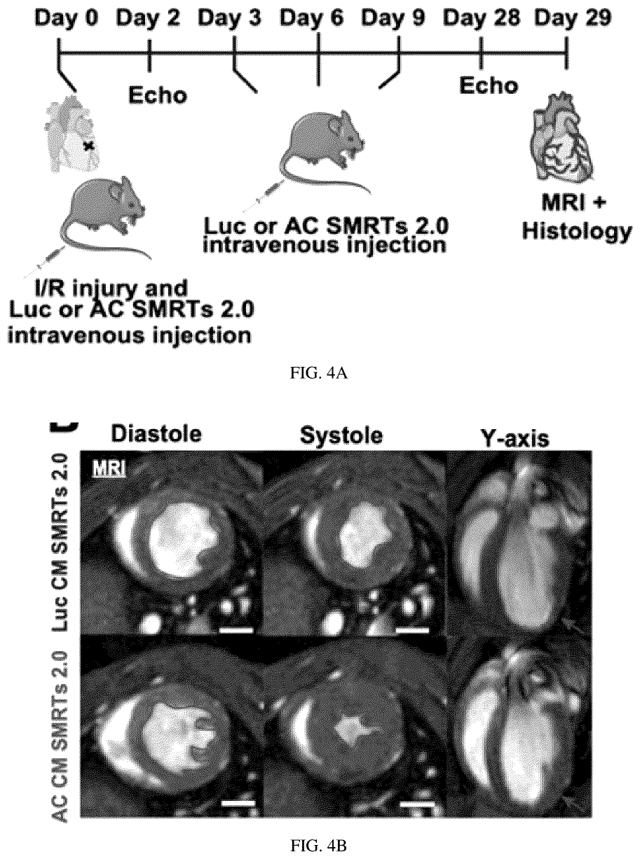

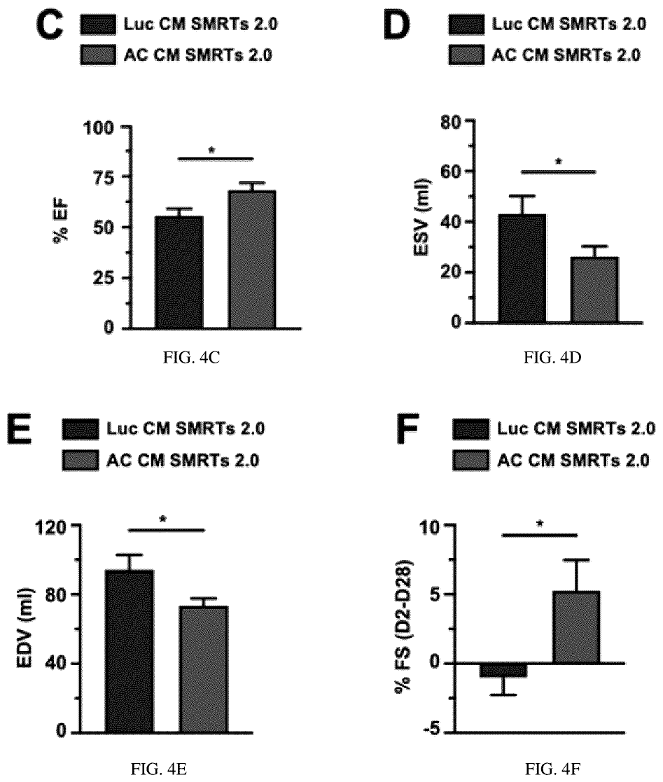

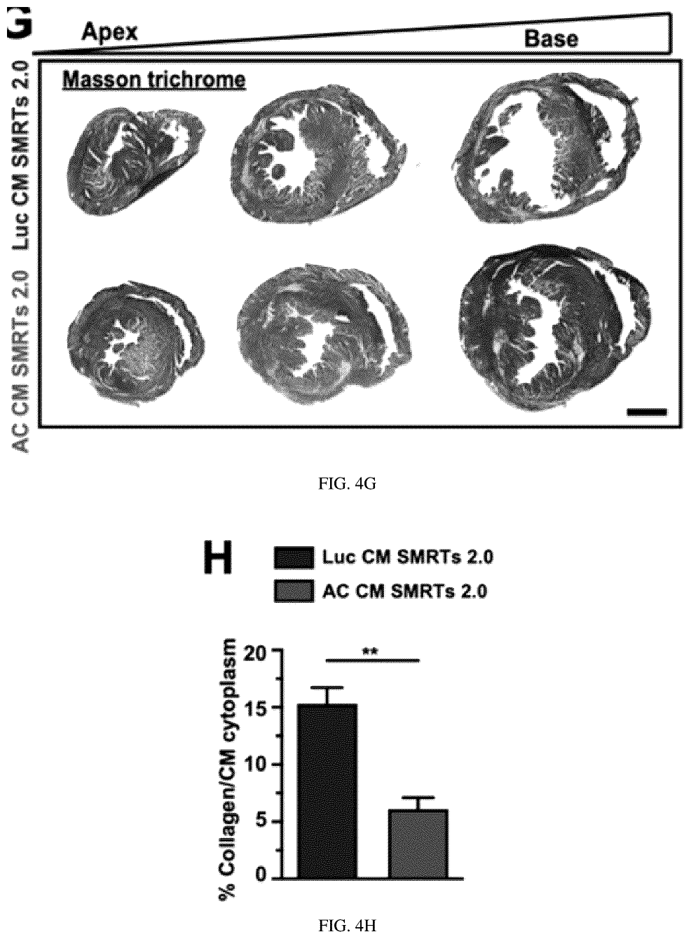

### Example 4: Cardiac Protection and Decrease Cardiac Remodeling Post Minimal Invasive Delivery of AC CM-SMRTs 2.0 in an I/R Injury Model (FIGS. 4A-4H)

FIG. 4A shows an example of a timeline to evaluate cardiac function and outcome in an acute cardiac I/R mouse model. B. Magnetic resonance imaging (MRI) assessments of left ventricular systolic function 28 days after I/R and 4 times intravenous delivery of Luc or AC SMRT 2.0 at three-day interval. Images depict left ventricular chamber (outlined) in diastole and systole. C-F. Percentage evaluation of ejection fraction (EF, C), end systolic volume (ESV, D) and end diastolic volume (EDV, E) measured by MRI (Luc SMRT 2.0 n=6, AC SMRT 2.0 n=8). F. Percentage of fractioning shortening differences, obtained by echocardiography (Echo), between day 2 (baseline) and day 28 post I/R (Luc SMRT 2.0 n=6, AC SMRT 2.0 n=7). G. Representative histological sections with Masson's trichrome staining to evaluate scar size 28 days post IR with different CM SMRT administration. H. quantification of % scar area (collagen+) in the left ventricle area 28 days post IR and different treatments (Luc SMRT 2.0 n=3, AC SMRT 2.0 n=3). Unpaired two-tailed t-test for C-F and H. Scale bar=1 mm.

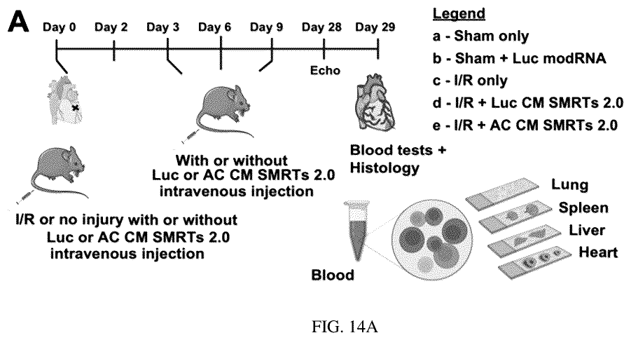

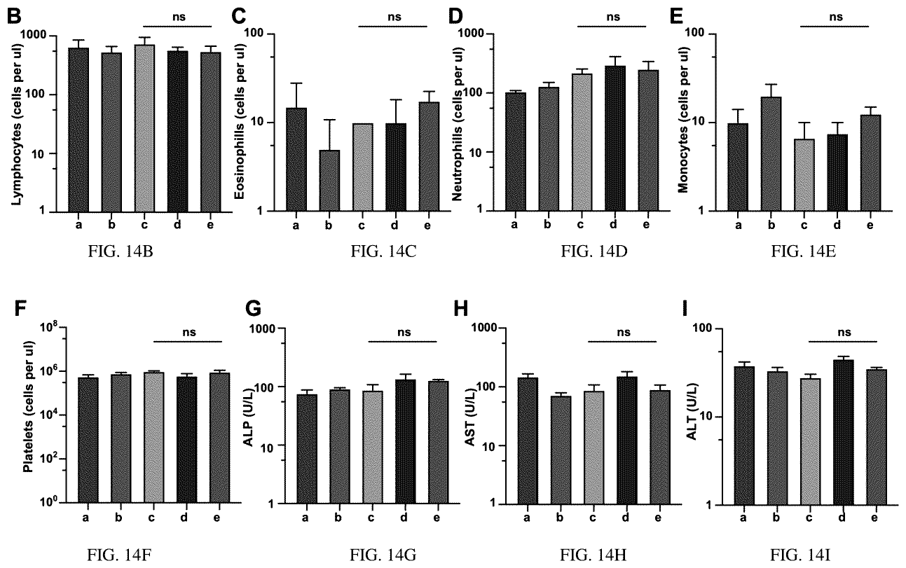

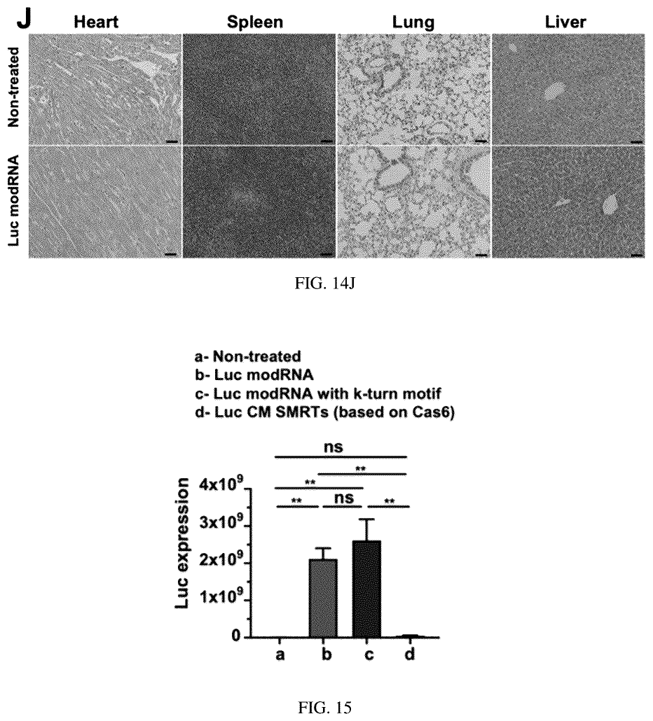

### Example 5: Safety Prolife of Different modRNA Delivered with Positively Charged Nanoparticles (FIGS. 14A-14I)

FIG. 14A shows an example of a timeline to evaluate impact on different organs in mouse body in different groups of mice with and without I/R surgery, injected intravenously with Luc modRNA, Luc SMRTs 2.0, AC SMRTs 2.0 (n=4). B-F. Immune response activation evaluated by complete blood count including lymphocytes, eosinophils, neutrophils, monocytes, and platelets respectively in different groups. G-I. Determination of liver enzyme levels alkaline phosphatase (ALP), aspartate aminotransferase (AST) or alanine transaminase (ALT) in five different group of mice. J. Representative hematoxylin and eosin-stained sections of heart, spleen, lung, and liver isolated from non-treated and Luc modRNA treated mice respectively to observe any bleeding or damage to the tissues post Luc modRNA and nanoparticle intravenous injections. Experiments were repeated four times independently. The values B-F are plotted in log scale. One-way ANOVA, Tukey's Multiple Comparison Test was used. Scale bar for J=25 μm.

### Example 6: FIG. 15 Shows Quantification of Bioluminescent IVIS Images if HEK Cells Transfected with modRNA Encoding CAS6 Carrying miR1-208 and Luc modRNA with Cas6 Hairpin (n=3). Unpaired t-Test was Used

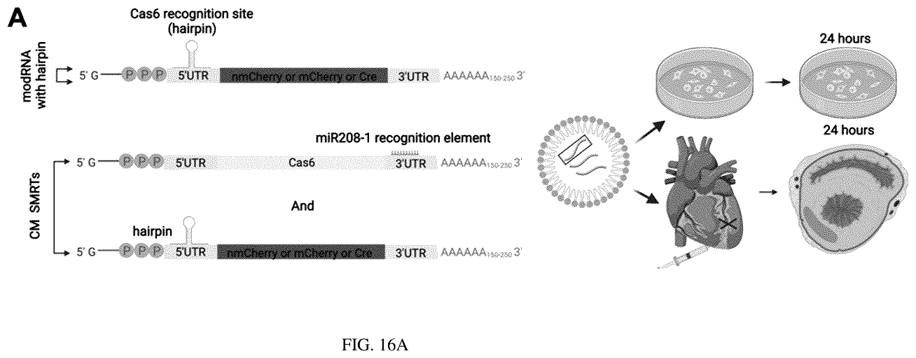

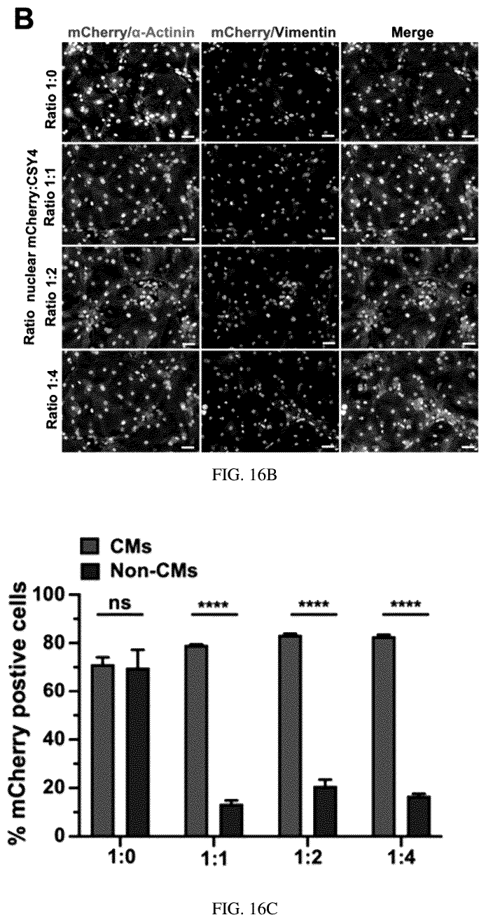

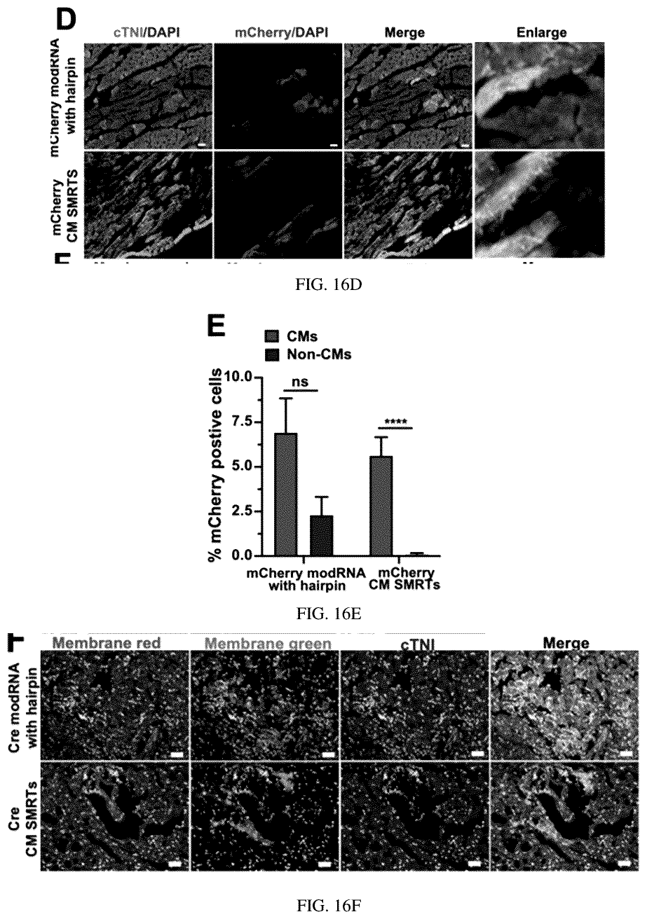

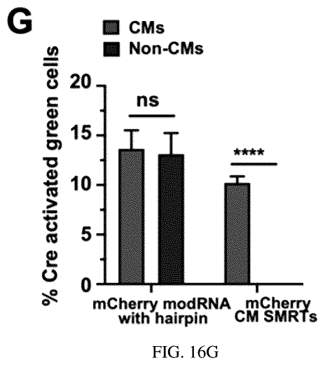

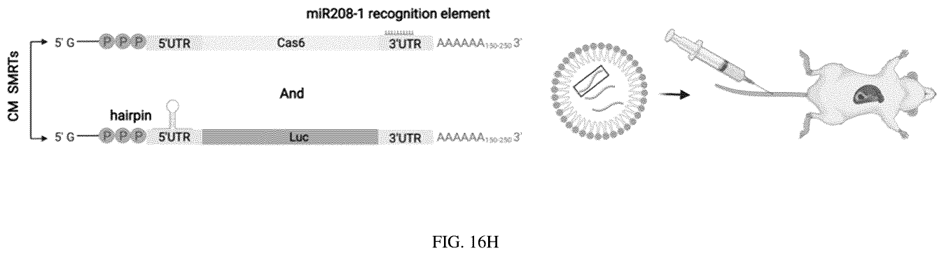

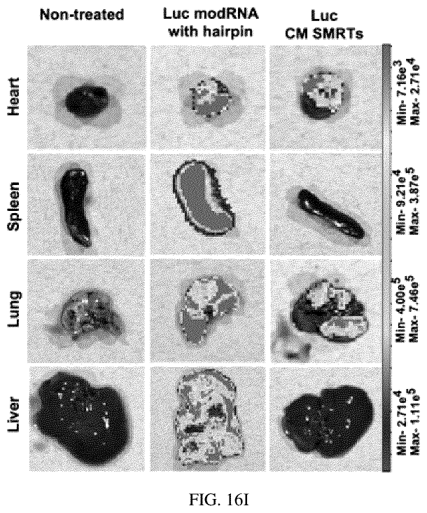

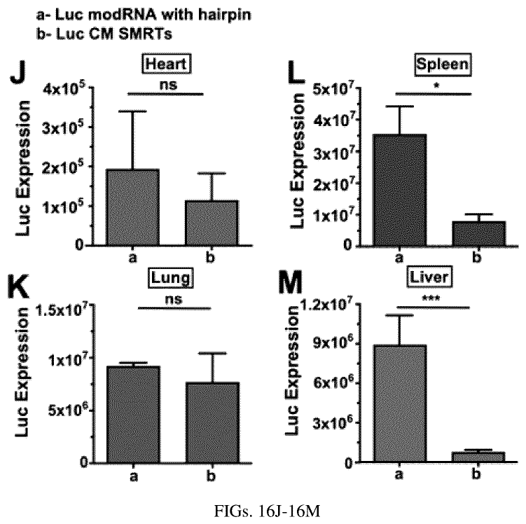

### Example 7: Evaluation of CM SMRTs Based on Cas6 Expression in the Heart, Following In Vitro and In Vivo Delivery (FIGS. 16A-16M)

FIG. 16A shows a schematic diagram of CM SMRTs which is structurally composed of two modRNAs, one containing the ribozyme Cas6 with an miR 208-1 recognition site and the other is made with gene of interest (nuclear mCherry or mCherry or Cre) with Cas6 recognition site (hairpin) downstream of 5′UTR. B. Representative images of neonatal mouse heart cells post-transfection with different ratios of mCherry modRNA with a Cas6 recognition site to Cas6 with an miR208-1 recognition element. CMs expressing α-actinin are presented in green while non-CMs are stained with the non-CM cell marker vimentin (blue). C. Quantification of B (n=3). D. Representative images of adult mouse heart post-transfection with mCherry with hairpin modRNA or mCherry modRNA with a Cas6 recognition site to Cas6 with an miR208-1 recognition element (CM SMRTs). Slides stained for CM marker (cTNI, green) cell nucleus (DAPI, blue) and mCherry (red). E. Percentage quantification of D (n=3). F. Representative images of Rosa^{26mTmG} adult mouse heart post-transfection with Cre with hairpin modRNA or CM SMRTs. Red cell membrane indicates untransfected cells while green cell membrane indicates successful translation of Cre in heart cells. Slides stained for CM marker (cTNI, blue) cell nucleus (DAPI, white). G. Percentage quantification of F (n=3).

### Example 8: Non-Limiting Examples of miR Recognition Elements that May Reduce modRNA Translation in Different Cells of Different Tissues Post I.V. Injection 9FIGS. 5A-5F)

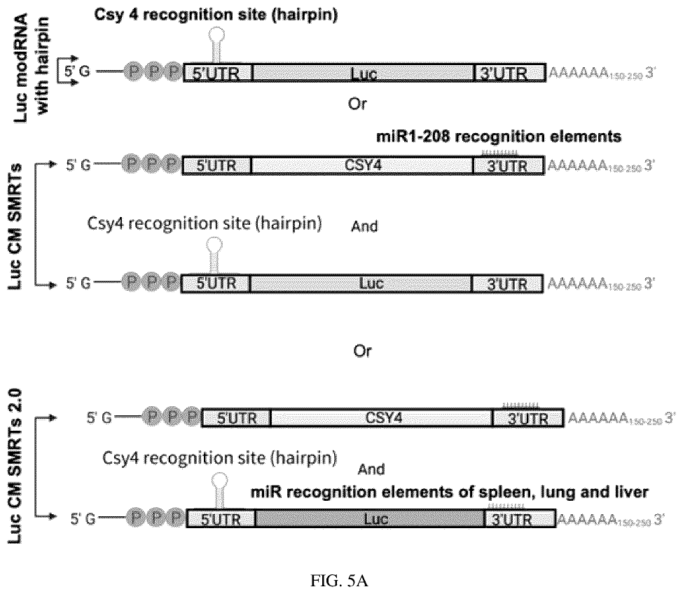

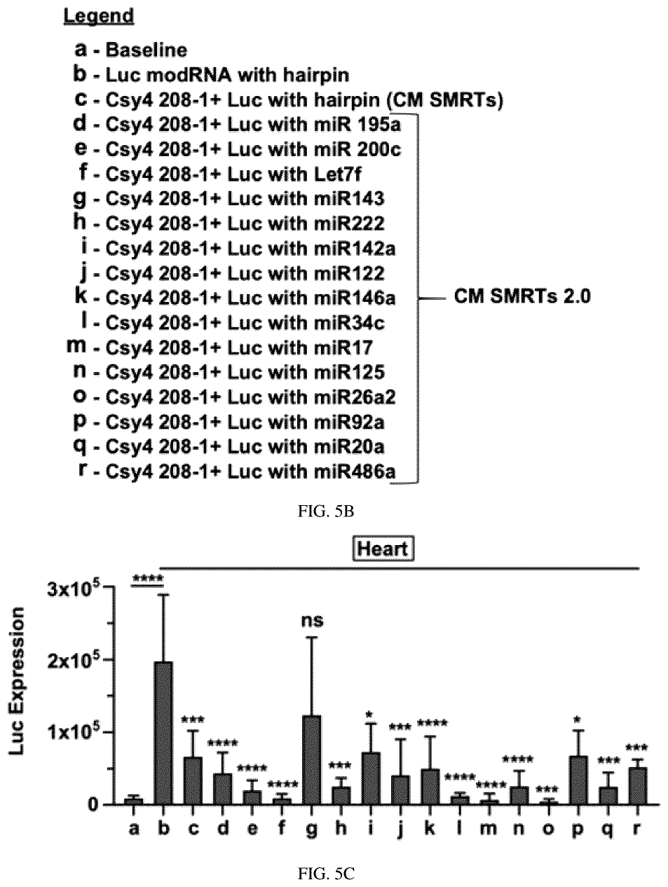

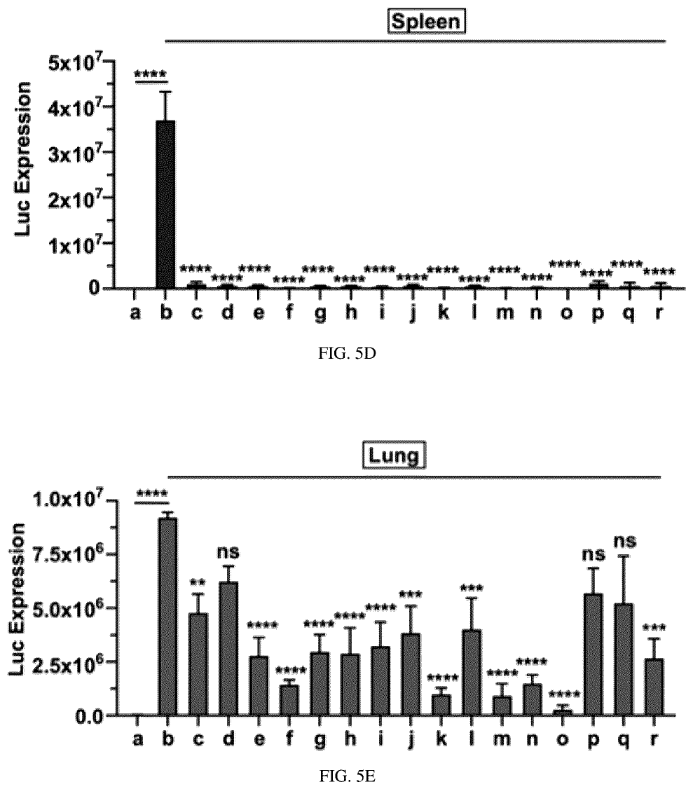

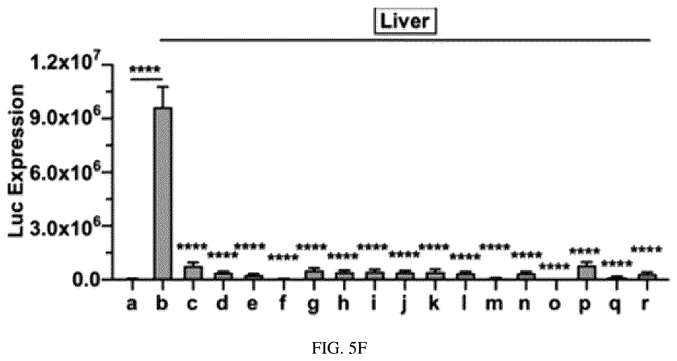

FIG. 5A shows a structural representation of a non-limiting example of a CM SMRT, co-transfection of Cas6 (also known as CSY4) with miR 208-1 recognition element and Luc modRNA containing hairpin, and identification of a non-limiting example of a CM SMRT 2.0 system containing Cas6 with miR 208-1 recognition element modRNA and Luc modRNA with hairpin downstream of 5′UTR and a miR recognition element in 3′UTR that can may reduce the gene expression in different cells of different tissues (B). C-F. Quantification of Luc expression in heart, spleen, lung and liver 24 hours post I.V injection of Luc containing different miR recognition elements against the above listed major organs. One-way ANOVA, Tukey's Multiple Comparison Test was used.

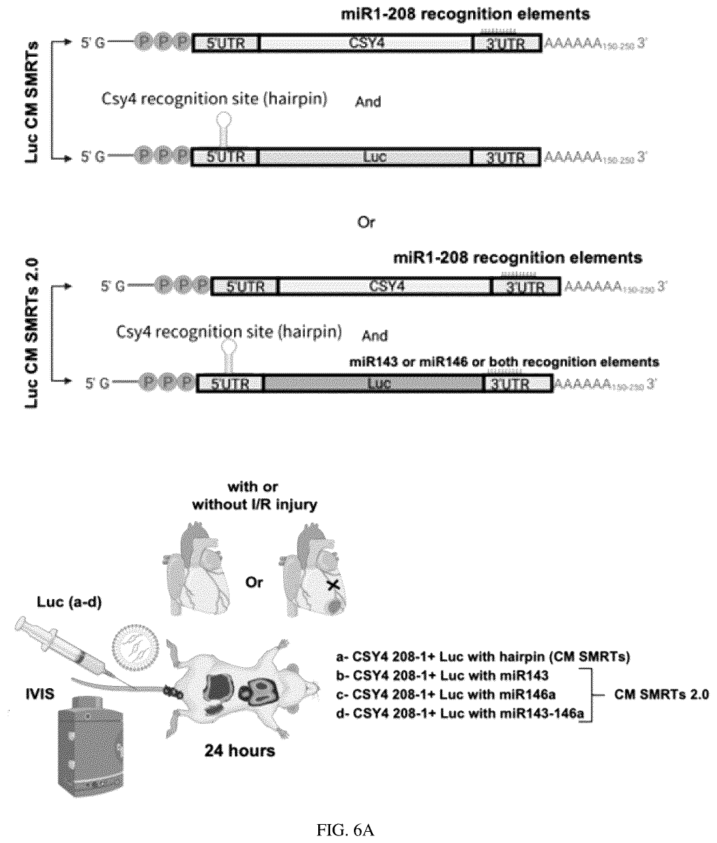

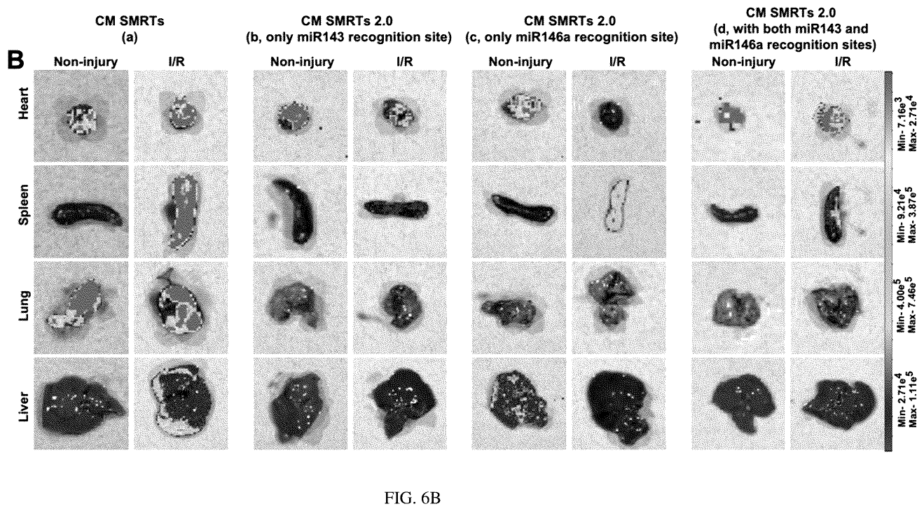

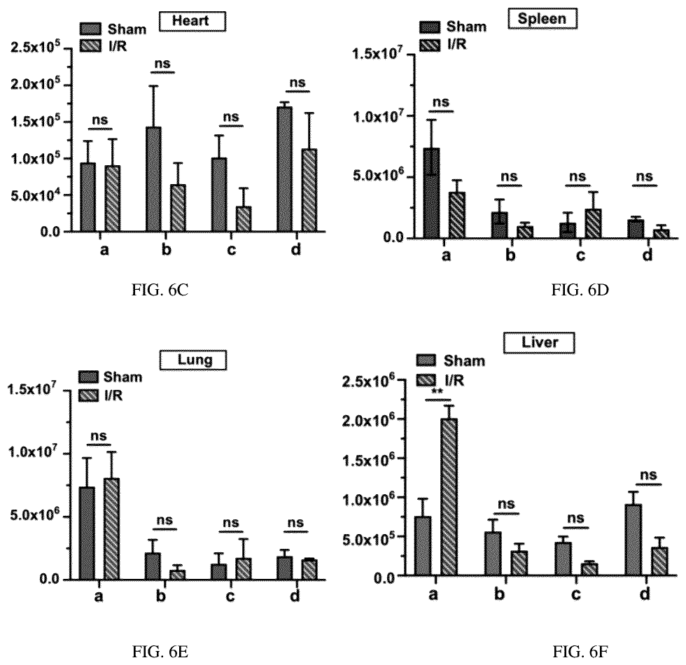

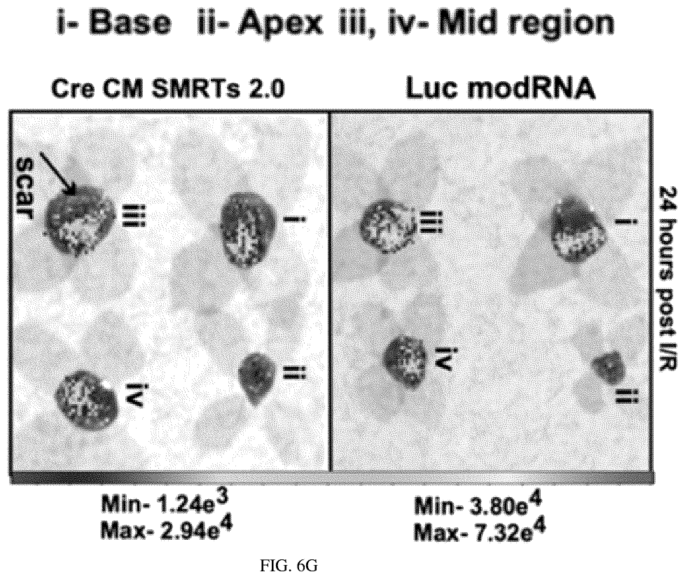

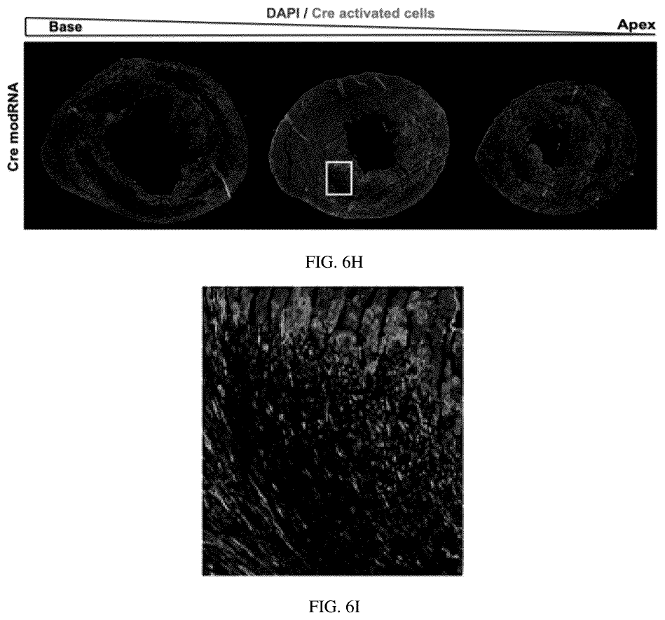

### Example 9: I.V Delivery of a Non-Limiting Examples of a Luc CM SMRT or of a CM SMRT 2.0 with or without Cardiac IR Injury (FIGS. 6A-6I)

FIG. 6A shows a non-limiting examples of a Luc CM SMRT or of a CM SMRT 2.0 containing recognition element miR-143 or miR146a, with or without cardiac I/R. B. Bioluminescence imaging of major organs 24 hours post I.V delivery of a CM SMRT or a CM SMRT 2.0 in non-injured and cardiac I/R injured mice. C-F. Quantification of total Luc expression in heart (C), lung (D), spleen (E), and liver (F), respectively, based on the experiment on B. G. Bioluminescence imaging of heart cross-section 24 hours post Luc with hairpin modRNA or a Luc SMRT 2.0 I.V injection. H. An example of a Cre CM SMRT 2.0 was injected I.V to evaluate biodistribution in hearts of ROSA26 knock-in Cre-reporter gene mice. Green cells indicate successful translation of Cre in heart cells. I. Enlarged image of heart section from H. One-way ANOVA, Tukey's Multiple Comparison Test was used.

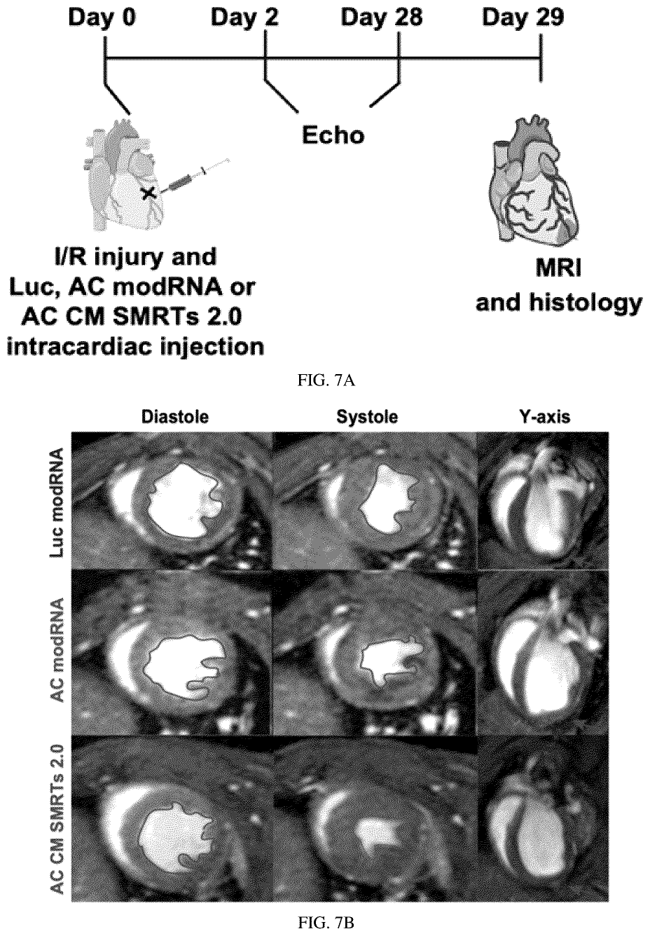

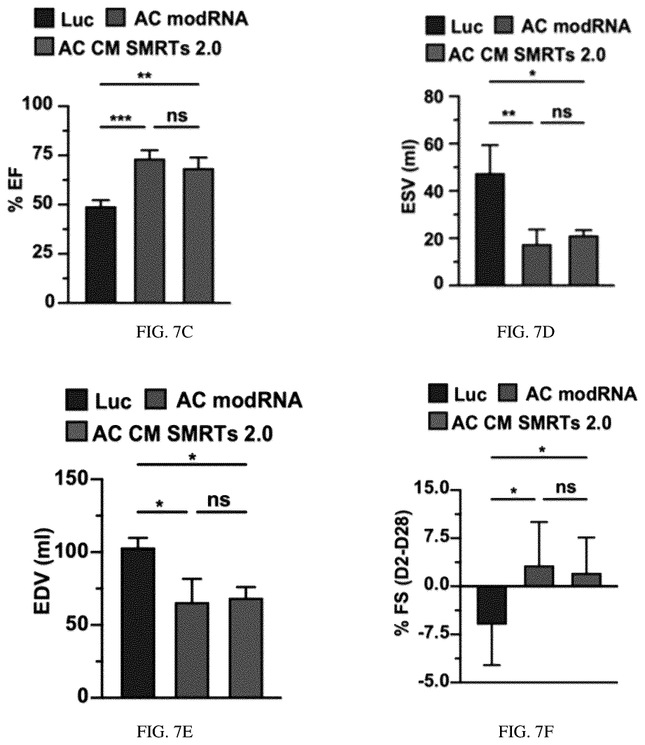

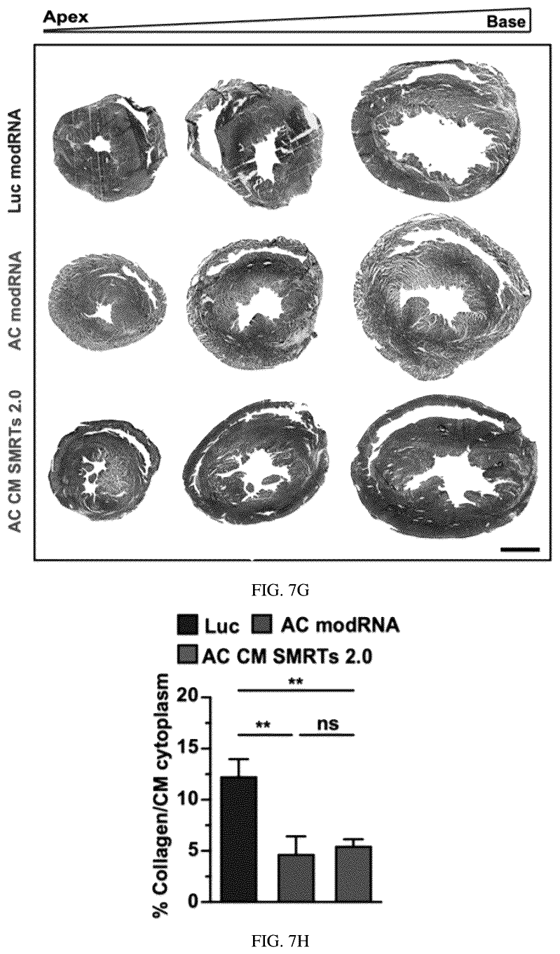

### Example 10: Intramyocardial Injection with an Example of Luc or of AC modRNA or with an Example of AC SMRT 2.0 in Preventing Cardiac Remodeling Post I/R Injury (FIGS. 7A-7H)

FIG. 7A shows an example of a timeline to evaluate cardiac function and outcome 28 days post I/R injury in CFW mice. B. MRI assessments of left ventricular systolic function 28 days after I/R and subsequent one-time intracardiac delivery at the time of ischemic injury. MRI images depict left ventricular chamber (outlined) in diastole and systole. C-E. MRI based percentage calculation of % ejection fraction (% EF, C), end systolic volume (ESV, D) and end diastolic volume (EDV, E) 28 days post-IR (Luc n=3, AC n=3 and AC SMRT 2.0 n=3). F. Echocardiography to determine changes in left ventricle % fractional shortening (% FS) between day 2 and day 28 post-IR (Luc n=8, AC n=5 and AC SMRT 2.0 n=7). G. Representative images of heart cross section stained with Masson's trichrome to evaluate cardiac scar tissue. H. Percentage quantification of G. (Luc n=3, AC n=3 and AC SMRT 2.0 n=3). One-way ANOVA, Tukey's Multiple Comparison Test was used. Scale bar for G=1 mm.

### Example 11: Further Genera Methods

Mouse Treatment

Adult C57B/6 mice (7-8-week-old) were lightly anesthetized with isoflurane and bleomycin hydrochloride [BAXTER (1 mg/kg) in 50 μl saline (0.9%) or vehicle (50 μl saline (0.9%)] was administered via oropharyngeal aspiration using a micropipette. 2 weeks later, mice were treated with modRNA encoding for Luc, TgfB, Pipk2c with Cas6 lung SMRTs. Post 21 days of modRNA treatment, lungs were isolated and snap frozen for downstream experiments.

Detection of Luciferase Expression Using IVIS System

Bioluminescence imaging of the transfected cells or I.V. injected mice was taken at (18h) in the IVIS system. To visualize cells expressing firefly luciferase invitro, D-luciferin was added to the cell-culture plate, and an image was taken in the IVIS system (IVIS Spectrum National Center for Research Resources [NCRR] S10-RR026561-01 at the Preclinical Small Imaging Core at Mount Sinai Medical Center). To visualize tissues expressing Luc in vivo, mice were anesthetized with isoflurane (Abbott Laboratories), and luciferin (150 mg/g body weight; Sigma) was injected intraperitoneally. Mice were imaged using an IVIS imaging system (IVIS Spectrum NCRR S10-RR026561-01 at the Preclinical Small Imaging Core at Mount Sinai Medical Center) every 2 min until the Luc signal reached a plateau. Imaging data were analyzed and quantified with Living Image software.

qPCR

RNA isolation and gene expression profiling using Real-Time PCR. Total RNA was isolated from the mouse lung tissue using the Quick-RNAMini prep Kit and reverse transcribed using ISCRIPT™ cDNA Synthesis Kit (Biorad) according to the manufacturer's instructions. Real-time qPCR analyses were performed on a Master cycler Real plex 4Sequence Detector (Eppendorf) using PerfeCTa SYBR GreenFastMix (Quanta Bio). Data were normalized to GAPDH expression; fold-changes in gene expression were determined by the ∂∂CT method and presented relative to an internal control.

Bone Marrow Isolation

Bone marrow was harvested from 8-12 wk C57BL/6 mice, as described. The total BMCs including the monocytes were resuspended at 106 cells/ml in Iscove's Modified Dulbecco's Medium/20% FBS and plated onto tissue culture plastic, with nonadherent cells removed after 4 hrs. The remaining adherent cells were cultured for 2 weeks and then split when still sub confluent for use in experimentation. Once the cultures were ready, the cells were transfected with mCherry modRNA in combination with Cas 6 modRNA containing miR146, miR20, miR 148 and miR223.

Immunostaining of Lung Tissue and Monocytes

Frozen Lung sections were rehydrated in PBS for 5 min, followed by permeabilization in PBS with 0.1% triton x 100 (PBST) for 7 min. Further, the samples were blocked with blocking serum (5% Donkey normal serum in PBST) for 2 hrs at room temperature, and primary antibody for alpha Smooth muscle actin diluted in blocking serum were added for overnight incubation at 4° C. Next day the slides were washed three times with PBST (5 min per wash), then incubated with a secondary antibody (Invitrogen, 1:200) diluted in PBST for 2 hours at room temperature. The samples were washed three times in PBST (5 min per wash) and stained with Hoechst 33342 (1 g/ml) diluted in PBST for 7 min. After five 4-min washes with PBST and one 4-min wash with tap water, slides were mounted with mounting medium (VECTASHIELD) for imaging. Stained slides were stored at 4° C. To immunostain adult bone marrow cells, cells were fixed on coverslips with 4% PFA for 15 min at room temperature and then washed three times with PBS. Following permeabilization with 0.5% Triton X in PBS for 10 min at room temperature, cells were blocked with 5% normal goat/donkey serum+0.5% Tween 20 for 30 min. Cover slips were incubated with primary antibody CD11b for 1 hr in a humid chamber at room temperature, followed by incubation with corresponding secondary antibodies conjugated to Alexa Fluor 488 as well as Hoechst 33342 staining for nuclei visualization.

4T1 Breast Cancer Specific Constructs

Breast cancer specific modRNA constructs contain two modRNA molecules: one carrying gene of interest (nGFP, Luc or therapeutic genes) is combined with Csy4 recognition element (hairpin) and the second contains gene coding for Csy4 endoribonuclease and cell specific micro RNA (miR) recognition element.

For systemic delivery, organ specific miR recognition elements were added on 3′ end of the modRNA construct containing gene of interest.

Cell Transfections and Imagining

4T1 breast cancer line cells (ATCC CRL-2539) were cultured using RPMI media supplemented with 10% FBS and pen-strep, BALBc Mouse Primary Mammary Epithelial Cells (MGEpith, Cell Biologics #BALB-5035) were cultured using supplementary media (Cell Biologics #M6621). For transfections, 40 000 cells per well were plated on 24 well cell culture plate one day before transfections. modRNA constructs carrying nGFP were used at 2.5 g of total modRNA per well for transfections with Lipofectamine 2000 (Invitrogen, #11668) following manufacturer's protocol. 24h after transfections cells were fixed with 4% PFA and stained with Hoechst. Fluorescence imaging was performed 24h later using Zeiss fluorescent microscope. Transfection efficiency was calculated as a percentage of nGFP+ cells of Hoechst+ cells.

Bioluminescent Imaging

100 000 4T1 breast cancer cells were inoculated into mammary gland pad of 8-10 weeks old BALBc female mice and grew for 10-14 days. For direct intratumor injections cancer specific constructs carrying Luc gene were delivered using jetRNA transfection reagent (Polyplus #101000021). 30 μg of total modRNA in 40 μl were used for intratumor and contralateral femur muscle injection. For systemic delivery, 30 μg of modRNA encapsulated in lipid nanoparticles were intravenously injected per mouse. 24h post injection Luc expression was evaluated using IVIS Spectrum In Vivo Imaging System (Perkin Elmer). Mice were injected intraperitoneally with 150 mg/kg body weight of D-Luciferin Potassium Salt (Perkin Elmer, #122799) and imaged every 2 min until reaching maximum luminescence. For ex vivo imagining, mice were sacrificed and bioluminescence of tumors and organs was measured.

Therapeutic Gene Evaluation

For therapeutic gene evaluation, 8-10 weeks old BALBc female mice were inoculated with 100 000 4T1 breast cancer cells. For intratumor injection, 7 days post inoculation mice were injected with therapeutic modRNA constructs and controls once a week with total 3 injections. For systemic delivery, 11 days post tumor inoculation mice were intravenously injected with therapeutic modRNA constructs and controls every 3 days with total of 5 injections. Tumor volume was measured twice a week using caliper. For systemic delivery, immunogenicity was assessed by measuring blood count of lymphocytes, neutrophils and monocytes. Liver toxicity was assessed by measuring amount of liver enzymes in the serum of treated mice at experimental endpoint.

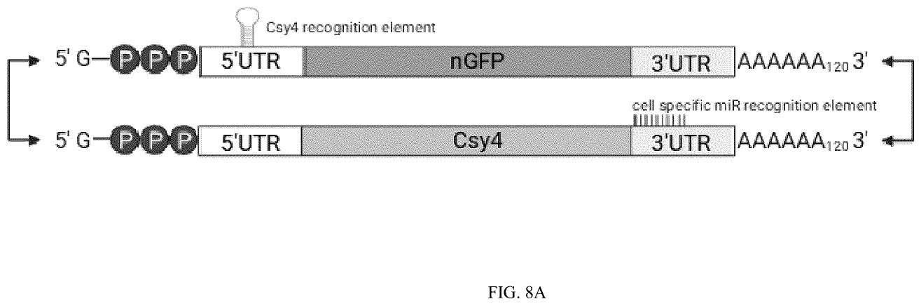

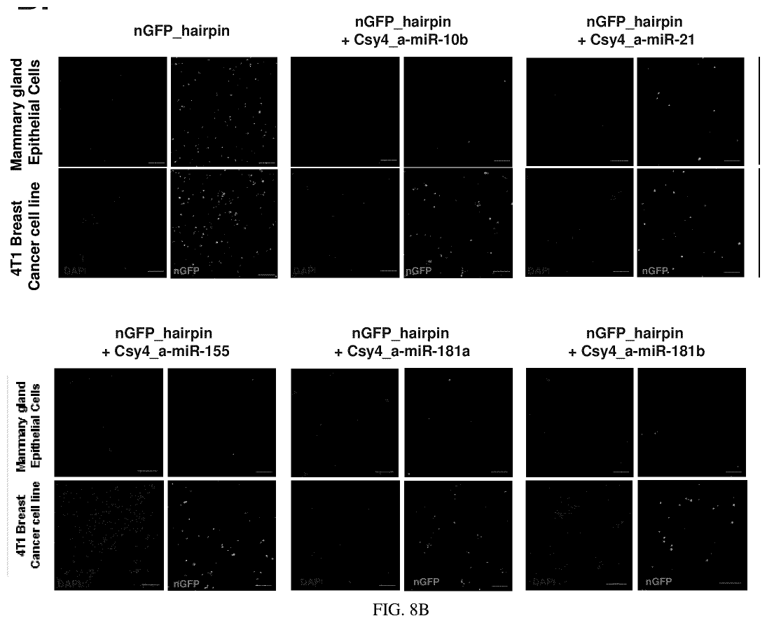

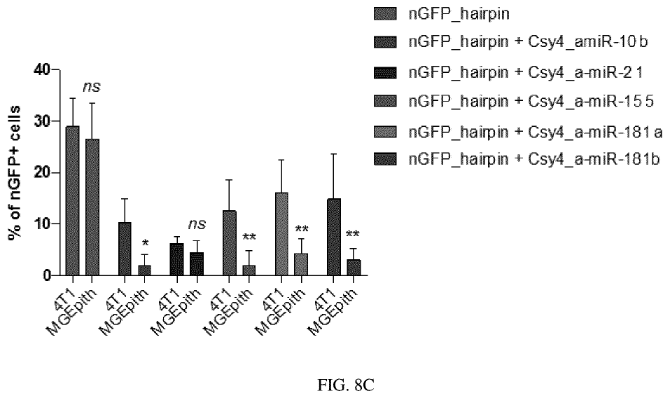

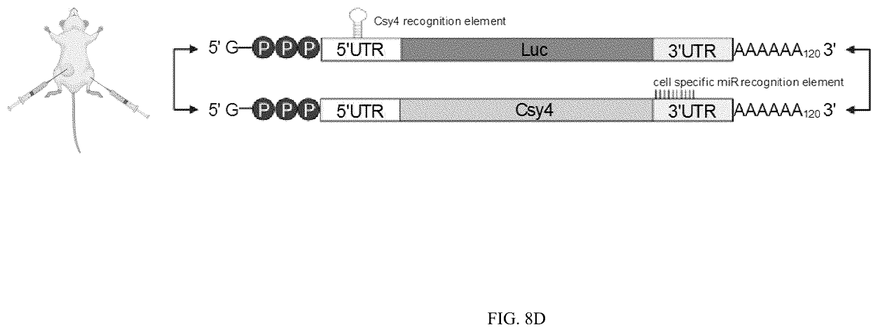

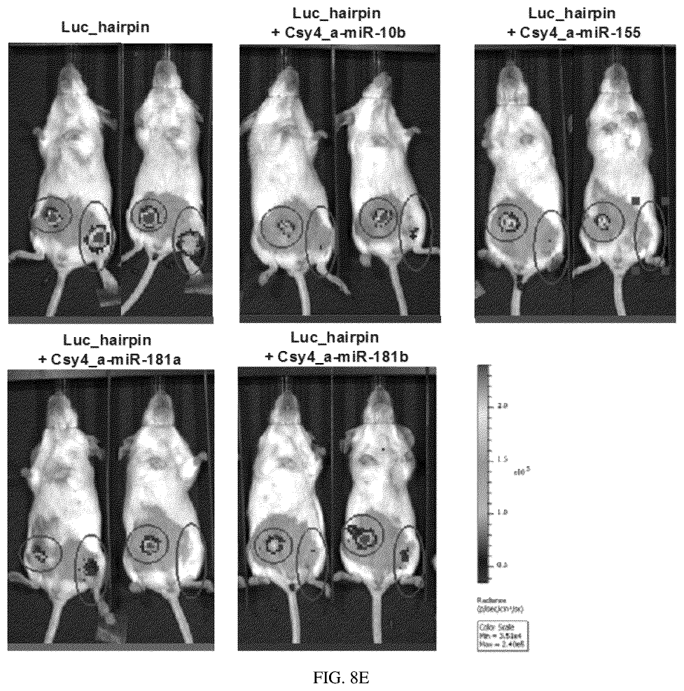

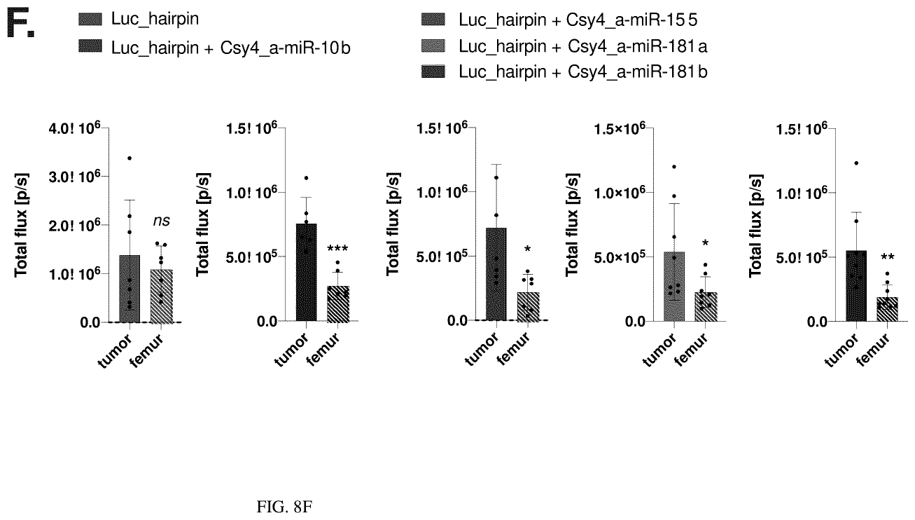

### Example 12: In Vitro and In Vivo Evaluation of 4T1 Breast Tumor SMRT (FIGS. 8A-8F)

FIG. 8A shows a non-limiting example of a SMRT construct for tumor cells in accordance with aspects of the present disclosure, including two modRNA molecules, one encoding a gene of interest (e.g., nGFP or Luc) and includes a Cas6 recognition element (hairpin) and the second contains gene coding for Cas6 endoribonuclease and a miR recognition element (for example, a cell-specific miR recognition element). B. Representative immunofluorescence images of mammary gland epithelial cell line and 4T1 breast cancer line cells transfected with modRNA constructs containing nGFP together with various breast tumor specific miR recognition elements to evaluate specificity of each construct. C. Quantification of tumor specific constructs was measured as ability to inhibit nGFP translation in mammary gland epithelial cell line while allowing nGFP expression in 4T1 breast cancer cell line, in vitro. E. modRNA constructs were injected into tumors as well as skeletal muscles of mice, and Luc expression was evaluated by bioluminescence imaging 24h later. F. Quantification of Luc expression in femur muscle versus tumor in using different modRNA constructs. Parametric t-test, n=6-9 in three independent experiments. Scale bar in =100 μm.

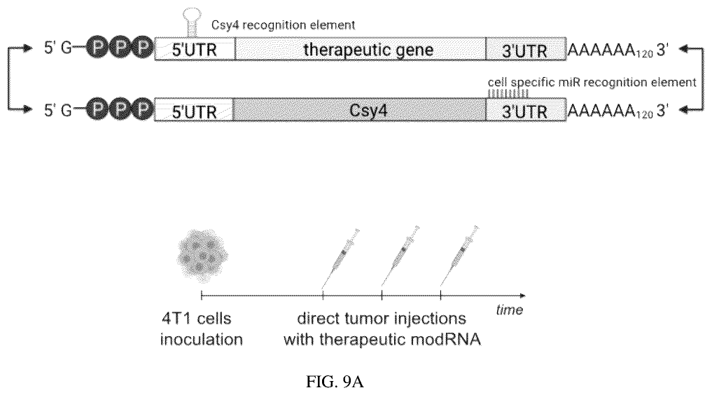

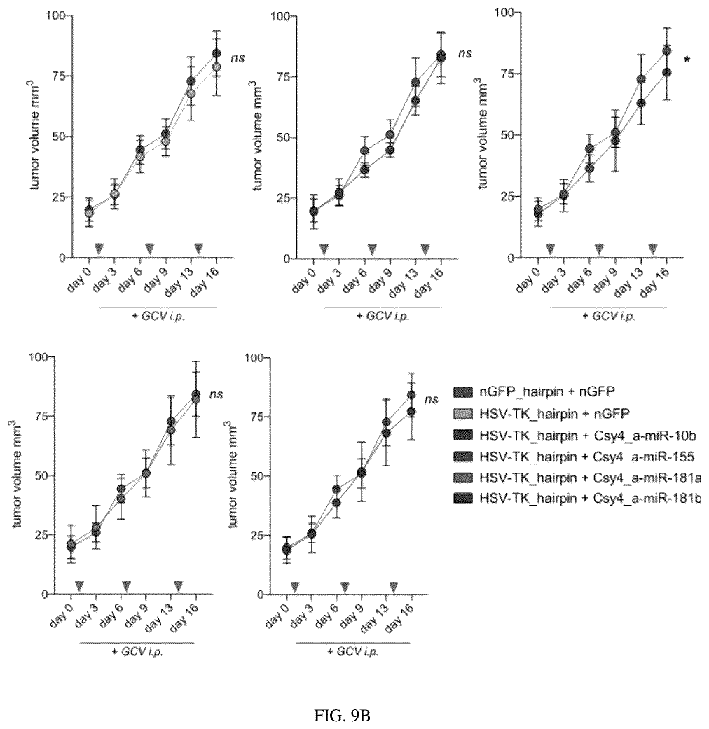

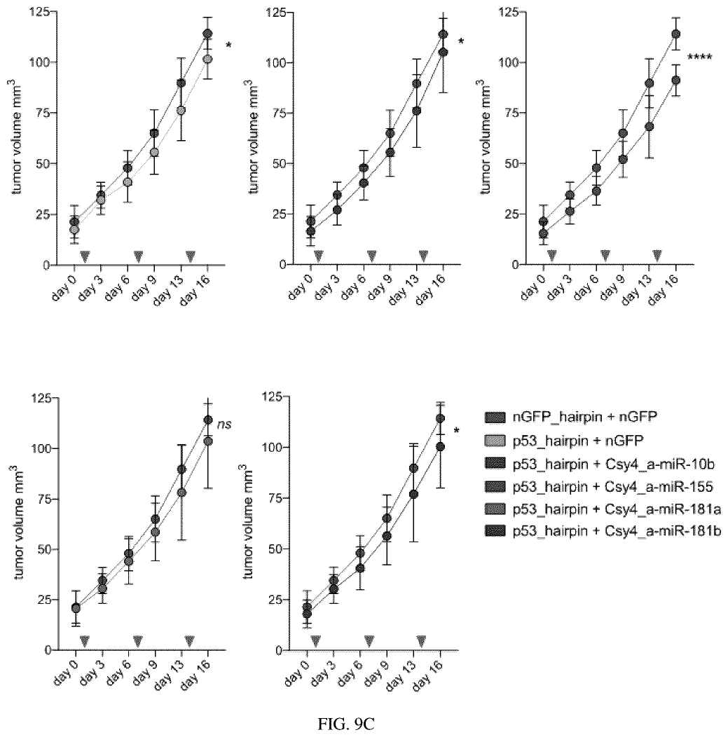

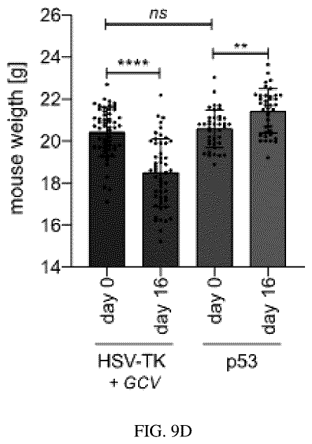

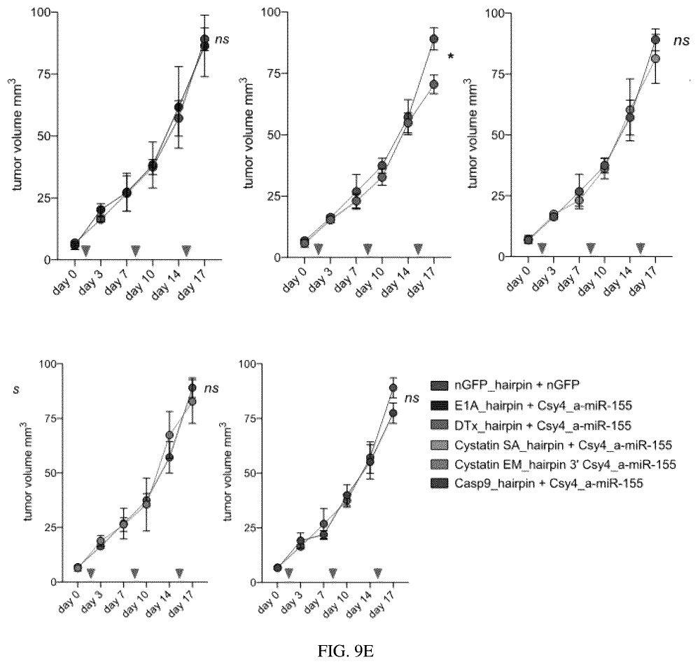

### Example 13: Evaluation of Tumor Growth Inhibition Using Various Tumor SMRTs Carrying Different Tumor Suppressor Genes (FIGS. 9A-9E)

FIG. 9A shows BALBc mice inoculated with 4T1 breast cancer line injected directly once a week with various tumor SMRT carrying different genes of interest encoding anti-tumor therapeutic proteins. Non-limiting examples of SMRT including herpes simplex virus thymidine kinase (HSV-TK) (B) or p53 (C) as a gene of interest were used together with daily i.p. injections of prodrug ganciclovir (GCV). D. Mouse body weight before the first injection and at the experimental endpoint in HSV-TK and p53 treated mice. E. Non-limiting examples of SMART including genes of interest coding for anti-tumor proteins were tested together with Cas6 with a miR-155 recognition element in accordance with aspects of the present disclosure. Two way ANOVA, n=6-9 in three independent experiments for B&C. One way ANOVA, n=39-54 in three independent experiments for D. Two way ANOVA, n=4 in one experiment for E

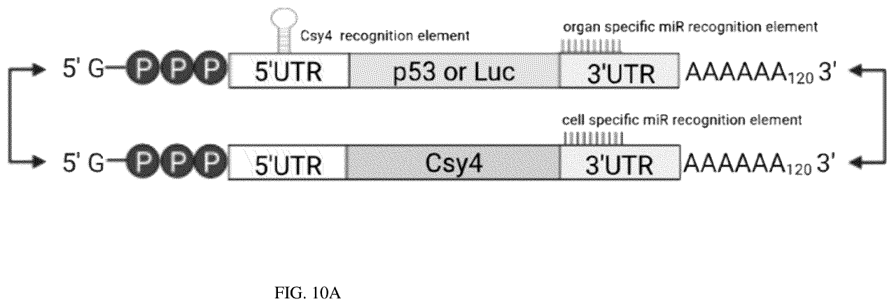

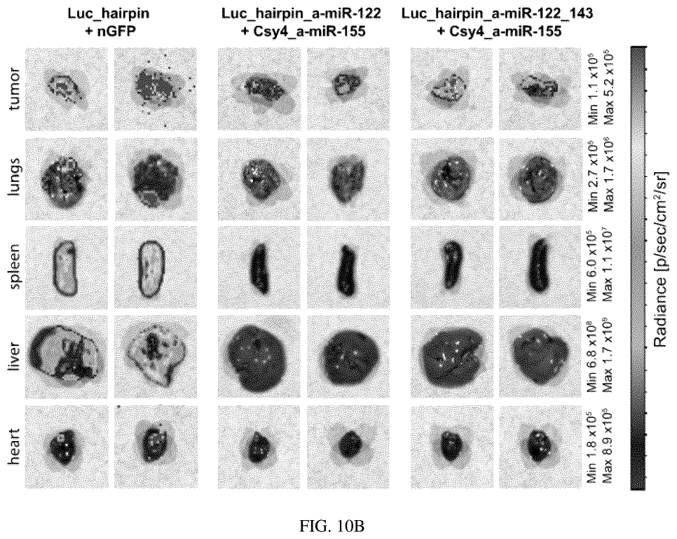

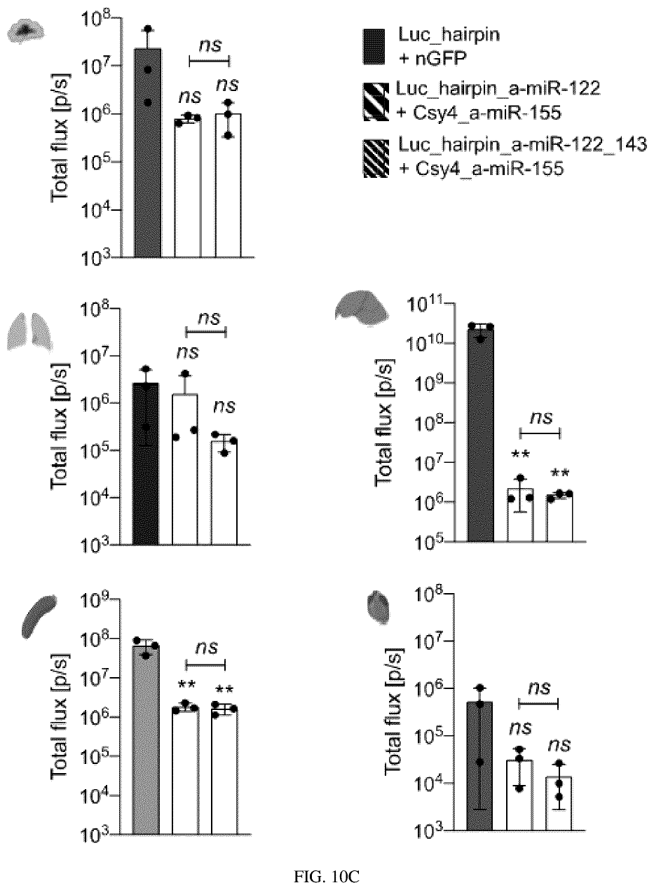

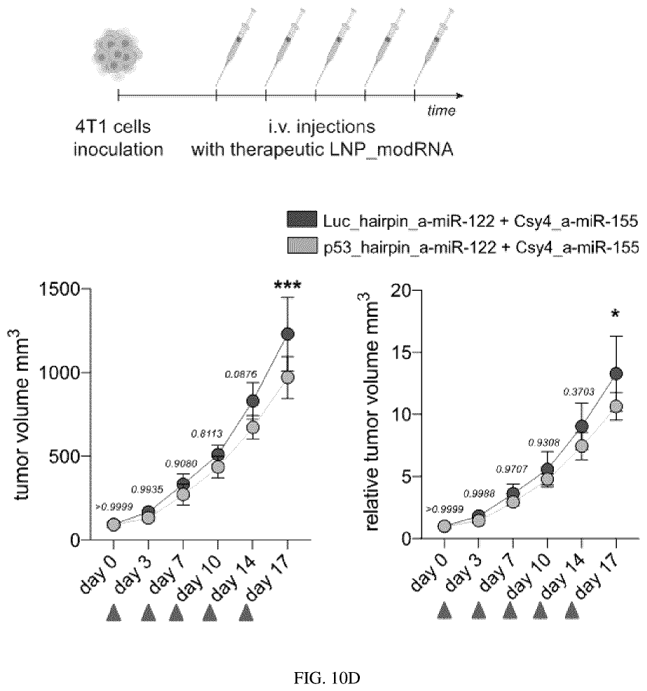

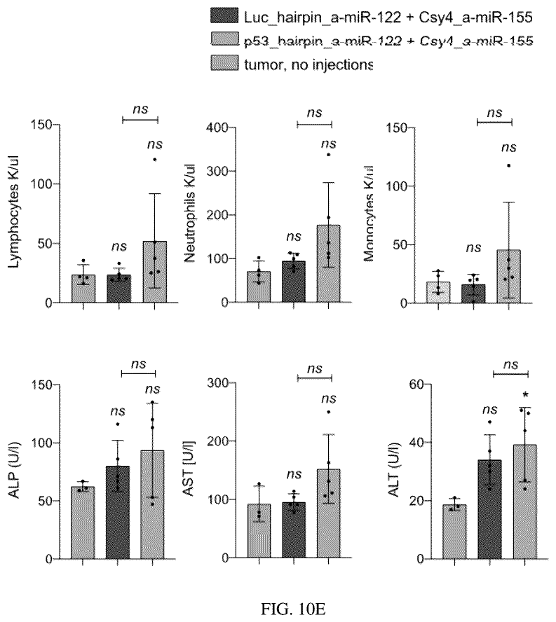

### Example 14: Minimal Invasive Delivery of Breast Tumor SMRT 2 (FIGS. 10A-10E)

FIG. 10A. shows examples of SMRT 2.0 constructs for minimal invasive delivery. To decrease transgene expression in non-target cells such as cells of spleen tissue or of liver tissue following intravenous injection in tumor bearing mice, miR recognition elements were included in the gene of interest modRNA (Luc or p53). B. Bioluminescent images of organs taken from mice 24h post I.V injection with different SMRT 2.0 constructs. C. Quantification of Luc protein expression in different tissues (tumor: top; lung and liver: middle left and right, respectively; spleen and heart: bottom left and right, respectively) 24h after I.V delivery of modRNA or examples of SMRT 2.0. D. I.V delivery of SMRT 2.0, including p53 as gene of interest with miR-122 (and Cas6 with miR-155), was performed every 3 days in 4T1-tumor bearing mice. Quantification of tumor growth both as an absolute and relative tumor volume. E. Analysis of blood cells and liver enzymes in mouse serum at the experimental endpoint. One way ANOVA, n=3 in one experiment for C. Two way ANOVA, n=5 in one experiment for D.

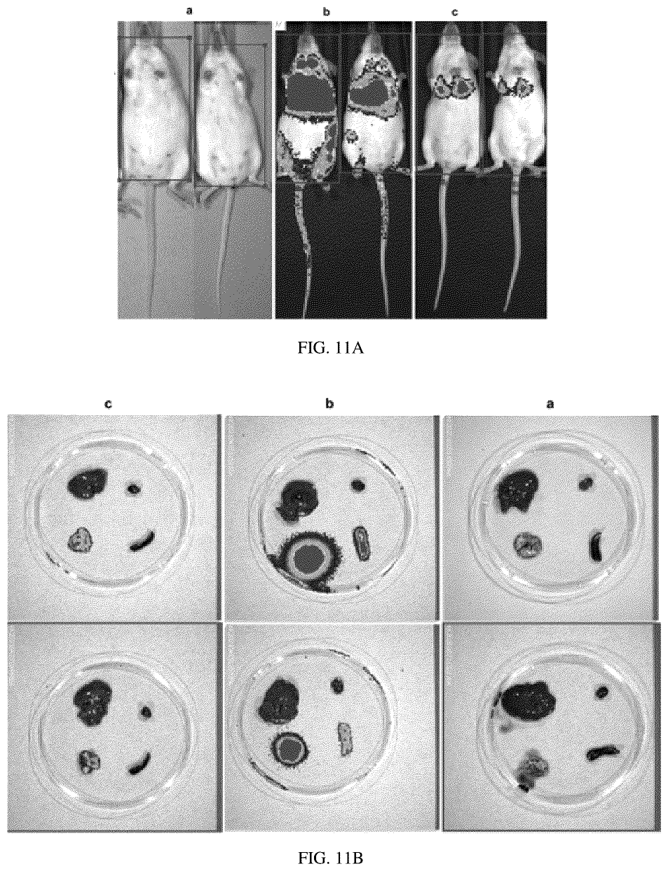

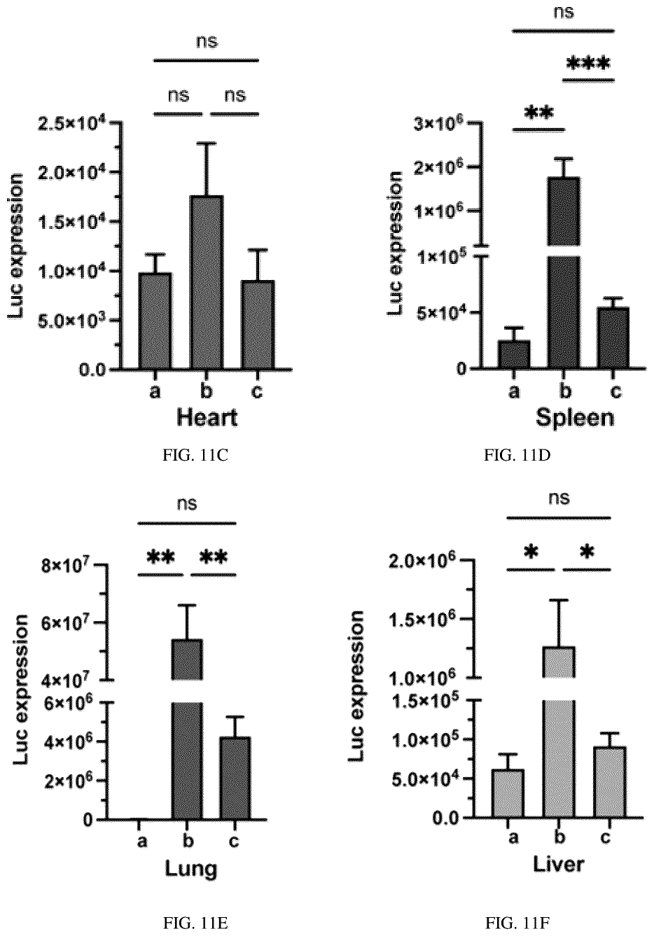

### Example 15: Intravenous Delivery of Luc and Lung SMRTs modRNA (FIGS. 11A-11F)

FIGS. 11A and 11B show in-vivo and Ex-vivo bioluminescence imaging of mouse received no injection (a), intravenous (IV) injection of Luc (b), or Luc of hairpin with Cas6 miR146a (c) encapsulated in positively charged nanoparticles, respectively. In B, ex vivo lung tissue is bottom left, others are heart, spleen, and liver. C-F. Quantification data representing total Luc expression in heart, spleen, lung and liver respectively. Results: I.V delivery of Luc modRNA in positively charged nanoparticles showed Luc expression in heart, spleen, lung, and liver, whereas SMRT injection showed robust expression in lung and significant lower expression in other organs.

### Example 16: Validation of the Lung Fibrosis Mouse Model by IT Injection of Bleomycin (Lung Fibrosis Evaluated 21 Days after Injection by CT Scan, Histology, and qPCR)

Fibrotic regions evaluated by micro CT comprising dense consolidation in lung lobes showed the evidence of fibrosis induced by bleomycin instillation. Further the presence of collagen (blue) staining by masson trichome and increased in expression of pro-fibrotic markers (TGFb and α-SMA) all indicated the presence of fibrosis in the lungs of mice treated post treatment with bleomycin (not shown).

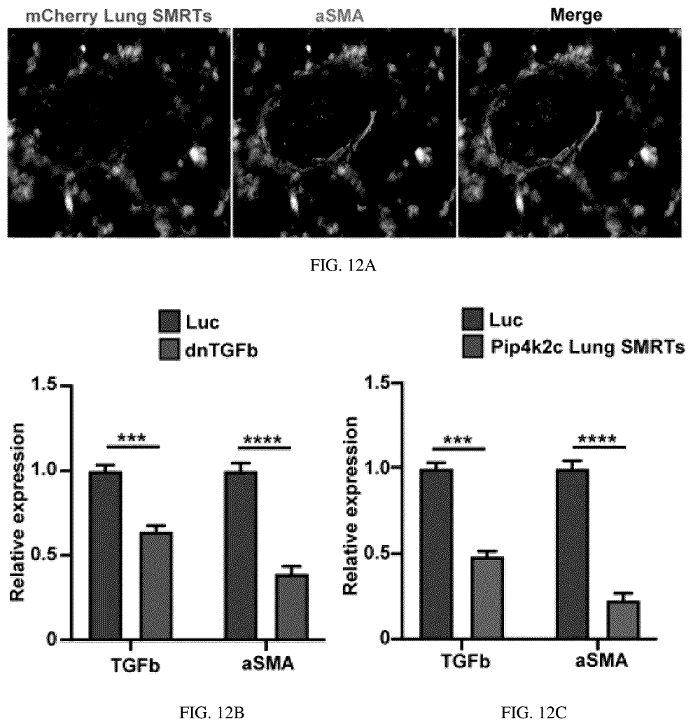

### Example 17: Pip4k2c Lung SMRTs or dnTGFb modRNA Reduces the Fibrosis in the Lung (FIGS. 12A-12C)

FIG. 12A shows representative immunostaining images of lungs stained for myofibroblasts (aSMA) and mCherry 24h post I.V delivery of mCherry SMRT. B. mRNA expression of fibrosis markers TGFb and aSMA 21 days post Luc, dnTGFb, and Pip4k2c SMRT modRNA in bleomycin treated mice, determined by qPCR. modRNA encapsulated in positively charged nanoparticles transfected myofibroblasts (aSMA positive) when delivered I.V. qPCR results revealed that I.V. administration of dnTGFb (positive control) and anti-fibrotic gene of interest Pip4k2c SMRT can decrease the level of lung fibrosis in bleomycin instilled mice as predicted by decrease in expression of TGFb and α-SMA. dnTGFb modRNA express in many cell types, whereas SMRT system with, in this example, miR-146a included in the Cas6 modRNA RNA molecule, enabled therapeutic genes such as pip4k2c to be expressed specifically in lung tissue and not in other organs.

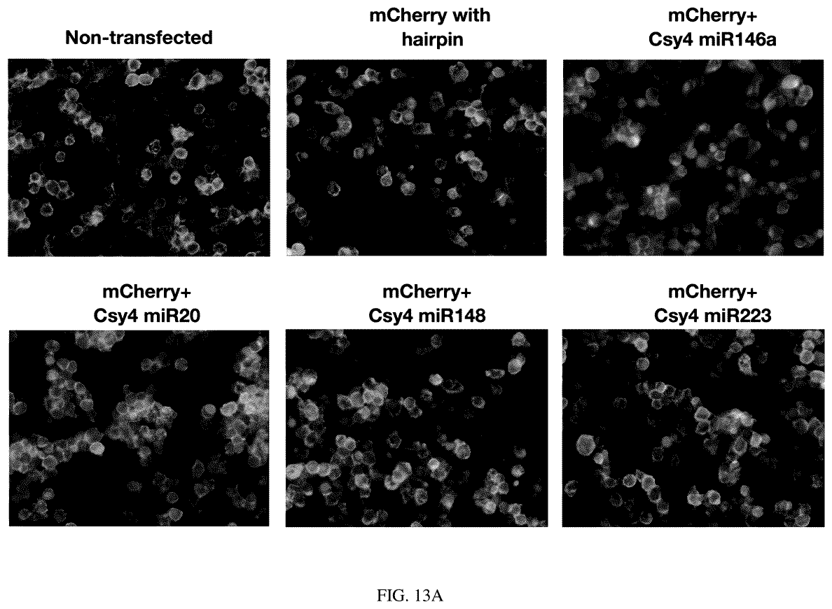

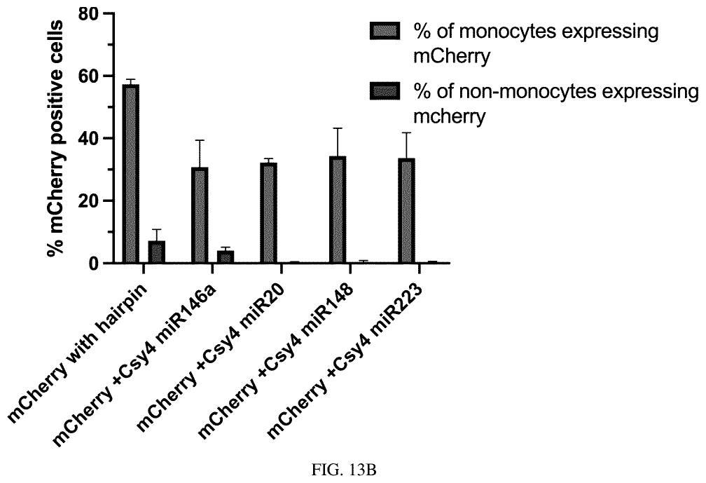

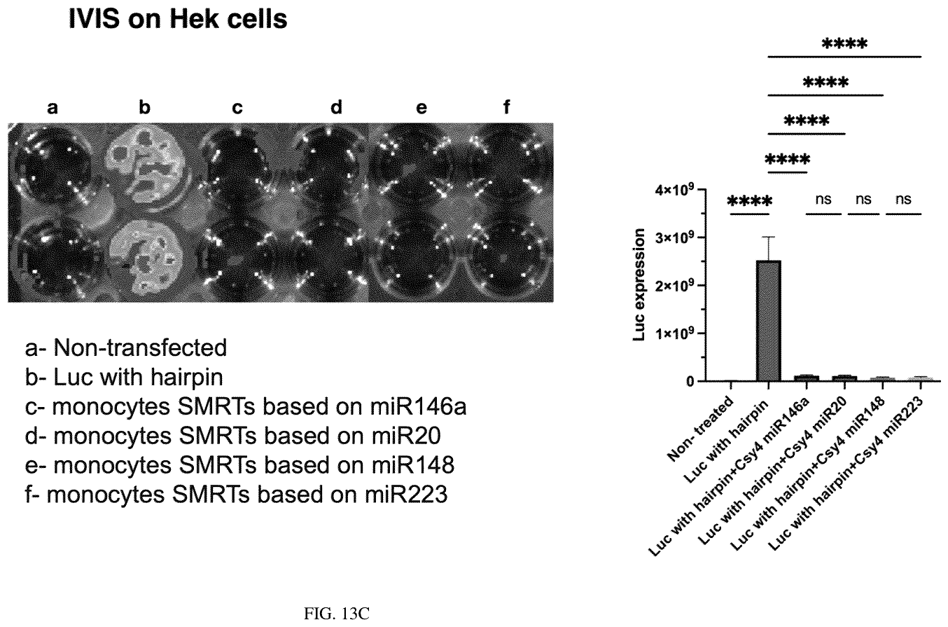

### Example 18: Minimally Invasive SMRTs Delivery to Monocytes (FIGS. 13A-13C)

FIG. 13A-C show in vitro expression of a gene of interest in monocytes (CD11b+) following in vitro treatment of adhered mouse bone marrow cells with a non-limiting example SMRT (Cas6 modRNA with miR146a, miR20, miR148, or miR223 response element, and mCherry modRNA with a Cas6-recognition hairpin). Adhered mouse bone marrow cells were transfected or not with nuclear mCherry with hairpin modRNA or with nuclear mCherry monocytes SMRT based on different miR recognition sites (miR146a, miR20, miR148, miR223). 24h later cells were fixed and stained for monocytes marker CD11b (green). B. Quantification of nuclear mCherry expression in monocytes (CD11b+) vs non-monocytes (CD11 b−) cells. miR20, miR148, miR223, yielded exclusive expression in monocytes (CD11b+). C. Bioluminescent image of Hek cells (cell line derived from human embryonic kidney cells, lacking monocytes) as a non-target cell, plated in 12 well plate transfected or not with Luc with hairpin modRNA or SMRT based (Cas6 modRNA with miR146a, miR20, miR148, miR223 recognition element, and Luc with Cas6 hairpin response element). Luc modRNA translate well in Hek cells but these examples of SMRT do not.

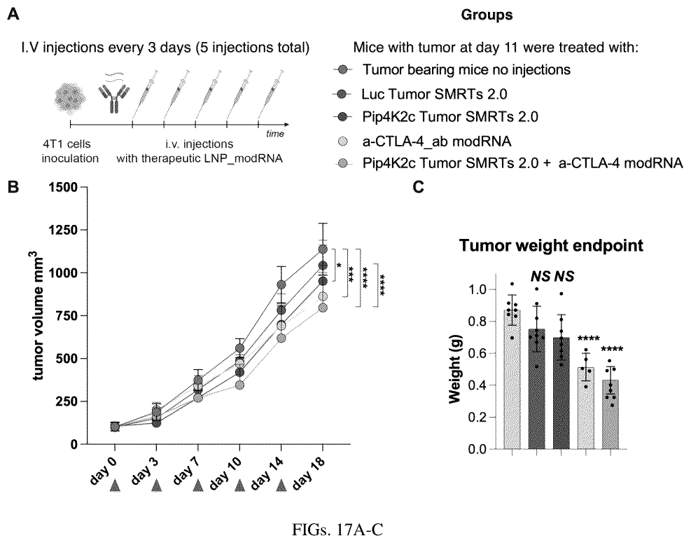

### Example 19: Delivery of Pip4k2c Breast Tumor SMRT 2.0 and Anti-CTLA-4 Antibody modRNA Significantly Reduce Tumor Volume and Weight (FIGS. 17A-17C)

FIG. 17A shows treatment regimen and groups. I.V. delivery of therapeutic Pip4k2c tumor SMRTs 2.0 with or without anti-CTLA-4 antibody modRNA (a-CTLA-4) was performed every 3 days in 4T1-tumor bearing mice. In this example, a first recombinant RNA molecule included an miR-155 recognition element it its 3′ UTR and encoded Cas6 and a second recombinant RNA molecule encoded Pip4k2c and included recognition elements for miR-122 and miR-143 in its 3′ UTR. B. Quantification of tumor volume and C. Tumor weight at endpoint. One way ANOVA, n=10.

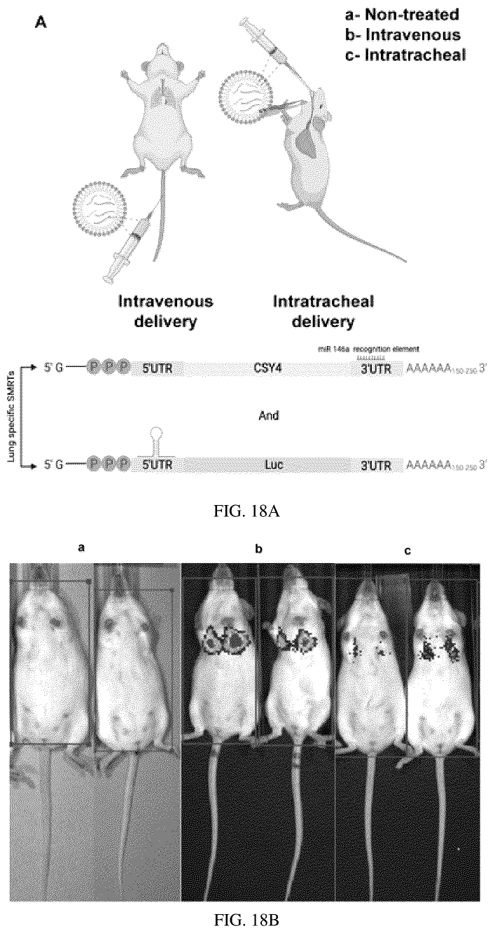

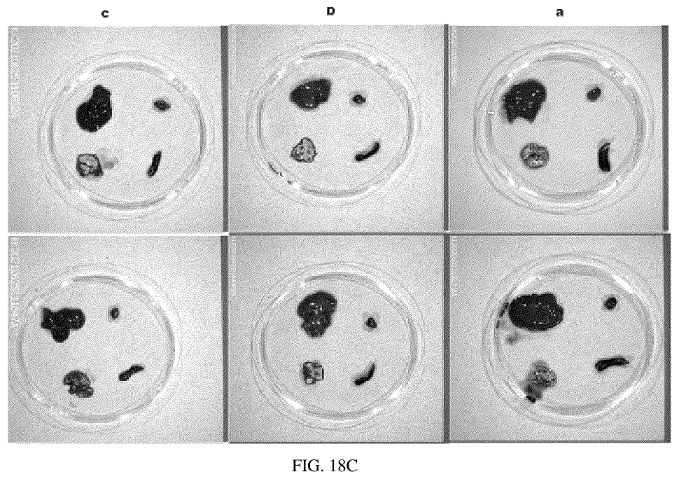

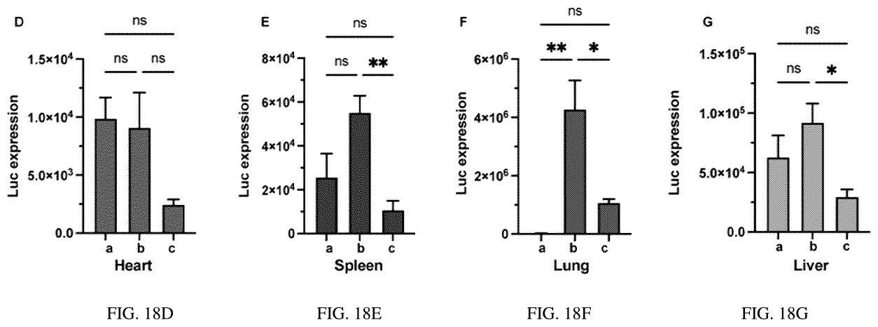

### Example 20: Comparison of Intravenous Vs Intratracheal Delivery of Lung SMRTs (FIGS. 18A-18G)

FIG. 18A shows a schematic diagram of minimal invasive delivery (intravenous (IV) or intratracheal (IT)) of Luc lung SMRTs. B&C. In-vivo and Ex-vivo bioluminescence imaging of mouse received no injection, I.V. or I.T of Luc Lung SMRTs, respectively. D-G. Quantification data representing total Luc expression in heart, spleen, lung and liver respectively 24 hours post no injection or I.V. or I.T Luc Lung SMRTs. I.V delivery of Luc Lung SMRTs modRNA showed significantly higher expression in all major organs including Lung compared to Luc expression evaluated by Luc Lung SMRTs intratracheal delivery.

Although some non-limiting examples have been depicted and described in detail herein, it will be apparent to those skilled in the relevant art that various modifications, additions, substitutions, and the like may be made without departing from the spirit of the present disclosure and these are therefore considered to be within the scope of the present disclosure as defined in the claims that follow.

It should be appreciated that all combinations of the foregoing concepts and additional concepts discussed in greater detail herein (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein and may be used to achieve the benefits and advantages described herein.

1. An expression regulatory system for expression of a gene of interest in a target cell, comprising
a recombinant first RNA molecule, comprising (i) a coding sequence for a Cas6 and (ii) a first microRNA (miR) recognition element in its 3′ UTR, wherein the first miR recognition element recognizes one or more first miR expressed in the target cell and binding of one or more of the one or more first miR to the first miR recognition element reduces translation of the translation suppressor, and
a recombinant second RNA molecule, comprising (i) a coding sequence for the gene of interest, (ii) a Cas6 recognition sequence, wherein binding of the translation-suppressor to the recognition sequence for the translation-suppressor reduces translation of the gene of interest, and (iii) a second miR recognition element in its 3′ UTR, wherein the second miR recognition element recognizes one or more second miR expressed in an off-target cell and binding of one or more of the one or more second miR to the second miR recognition element reduces translation of the gene of interest; wherein
one or both of the first RNA molecule and the second RNA molecule comprises one or more of pseudouridine substituted for uridine, 5-methylcytidine substituted for cytidine, and an anti-reverse cap analog.

2-6. (canceled)

7. The expression regulatory system of claim 1, wherein the target cell is a heart tissue cell, a lung tissue cell, a liver tissue cell, a spleen tissue cell, or a tumor cell.

8-22. (canceled)

23. The expression regulatory system of claim 1, further comprising a nanoparticle, wherein the nanoparticle comprises the first RNA molecule and the second RNA molecule.

24. The expression regulatory system of claim 23, wherein the nanoparticle comprises any one or more of a liposome nanoparticle, a gold nanoparticle, an iron nanoparticle, a poly lactic-co-glycolic acid nanoparticle, and a viral vector.

25. The expression regulatory system of claim 23, wherein the nanoparticle comprises a positive charge, a negative charge, or a neutral charge.

26. The expression regulatory system of claim 1, wherein the second miR recognition element includes one or more of a miR-195a recognition element, a miR-200c recognition element, a miR-Let7f recognition element, a miR-143 recognition element, a miR-222 recognition element, a miR-142a recognition element, a miR-122 recognition element, a miR-146a recognition element, a miR-34c recognition element, a miR-17 recognition element, a miR-125 recognition element, a miR-26a2 recognition element, a miR-92a recognition element, a miR-20a recognition element, and a miR-486a recognition element, a miR-146a recognition element, and any combination of two or more of the foregoing.

27. The expression regulatory system of claim 1, wherein the first miR recognition element comprises one or both of a miR-1 recognition element and a miR-208 recognition element.

28. The expression regulatory system of claim 27, wherein the second miR recognition element comprises one or both of a miR-143 recognition element and a miR-146a recognition element.

29. (canceled)

30. (canceled)

31. The expression regulatory system of claim 1, wherein the target cell is a heart tissue cell.

32. The expression regulatory system of claim 27, wherein the one or more off-target cell is selected from one or more of a non-cardiomyocyte heart tissue cell, a lung tissue cell, a liver tissue cell, and a spleen tissue cell.

33. The expression regulatory system of claim 27, wherein the target cell is a cardiomyocyte.

34. (canceled)

35. The expression regulatory system of claim 27, further comprising a nanoparticle, wherein the nanoparticle comprises the first RNA molecule, the second RNA molecule, and a positive charge.

36. The expression regulatory system of claim 27, wherein the gene of interest encodes an acid ceramidase a type 2 phosphatidylinositol-5-phosphate 4-kinase gamma a Lin28, a Pkm2, or a Cyclin D2.

37. The expression regulatory system of claim 1, wherein the first miR recognition element comprises one or more of a miR-155 recognition element, a miR10b recognition element, a miR18-la recognition element, and miR-181b recognition element.

38. The expression regulatory system of claim 37, wherein the second miR recognition element comprises one or more of a miR143 recognition element and a miR122 recognition element.

39-43. (canceled)

44. The expression regulatory system of claim 37, wherein the target cell comprises a tumor cell.

45. The expression regulatory system of claim 44, wherein the tumor cell comprises a breast tumor cell.

46. (canceled)

47. (canceled)

48. The expression regulatory system of claim 37, wherein the gene of interest encodes a p53 protein, a Herpes Simplex Virus type 1 thymidine kinase, a deltex protein, an E1A protein, a cystatin SA protein, a cystatin E/M protein, or a caspase 9 protein.

49. The expression regulatory system of claim 37, wherein the gene of interest encodes a tumor-suppressor protein.

50. A method, comprising administering the expression regulatory system of claim 1 to a subject, wherein (i) the subject suffered a myocardial infarction or suffers from heart failure, the first miR recognition element comprises one or both of a miR-1 recognition element and a miR-208 recognition element, the second miR recognition element comprises one or both of a miR-143 recognition element and a miR-146a recognition element, and the gene of interest encodes one or more of an acid ceramidase, a type 2 phosphatidylinositol-5-phosphate 4-kinase gamma a Lin28, a Pkm2, and a Cyclin D2 or (ii) the subject suffers from cancer, the first miR recognition element comprises one or more of a miR-155 recognition element, a miR10b recognition element, a miR181a recognition element, and a miR-181b recognition element, the second miR recognition element comprises one or more of a miR143 recognition element and a miR122 recognition element, and the gene of interest encodes one or more of a p53 protein, a Herpes Simplex Virus type 1 thymidine kinase, a deltex protein, an E1A protein, a cystatin SA protein, a cystatin E/M protein, a caspase 9 protein, and a tumor suppressor protein.

51-205. (canceled)


SEQUENCE LISTING (56 sequences)

SEQ ID NO: 1 - RNA, 564 nt, unidentified
  source 1..564: mol_type = other RNA
atggaccactacctcgacattcgcttgcgaccggacccggaatttcccccggcgcaactcatgagcgtgctcttcggcaagctccaccaggccctggtggcacagggcggggacaggatcggcgtgagcttccccgacctcgacgaaagccgctcccggctgggcgagcgcctgcgcattcatgcctcggcggacgaccttcgtgccctgctcgcccggccctggctggaagggttgcgggaccatctgcaattcggagaaccggcagtcgtgcctcaccccacaccgtaccgtcaggtcagtcgggttcaggcgaaaagcaatccggaacgcctgcggcggcggctcatgcgccggcacgatctgagtgaggaggaggctcggaaacgcattcccgatacggtcgcgagagccttggacctgcccttcgtcacgctacgcagccagagcaccggacagcacttccgtctcttcatccgccacgggccgttgcaggtgacggcagaggaaggaggattcacctgttacgggttgagcaaaggaggtttcgttccctggttctga

SEQ ID NO: 2 - RNA, 360 nt, unidentified
  source 1..360: mol_type = other RNA
atgtacgtgagatttgaggttcctgaggacatgcagaacgaagctctgagtctgctggagaaggttagggagagcggtaaggtaaagaaaggtaccaacgagacgacaaaggctgtggagaggggactggcaaagctcgtttacatcgcagaggatgttgacccgcctgagatcgttgctcatctgcccctcctctgcgaggagaagaatgtgccgtacatttacgttaaaagcaagaacgaccttggaagggctgtgggcattgaggtgccatgcgcttcggcagcgataatcaacgagggagagctgagaaaggagcttggaagccttgtggagaagattaaaggccttcagaagtaa

SEQ ID NO: 3 - RNA, 435 nt, unidentified
  source 1..435: mol_type = other RNA
atgtcaggtgatgaggctgcccctgttgttgttcctccccccgttgctgagccagcggccatcccagaggacatggacttaatgactgcattggagttgactcttaggaaagctcgtgcttatggtggtgttgttcgtggtctccatgagtgtgctaagcttattgagaagcgtgtggctcaactcgttgtcttggctgaagactgcaaccagcctgattatgtcaagcttgtgaaggctctctgtgctgatcacgaagtcaggttgctaacagttccaagtgccaagacccttggcgaatgggctggtctctgcaagattgattctgagggtaatgccaggaaggttgttggatgctcatgtcttgttgtcaaggacttcggcgaggagacaactgccctcagtatcgtcaataagcatattgcttctcaataa

SEQ ID NO: 4 - RNA, 28 nt, unidentified
  source 1..28: mol_type = other RNA
gttcactgccgtataggcagctaagaaa

SEQ ID NO: 5 - RNA, 63 nt, unidentified
  source 1..63: mol_type = other RNA
aaaggtgggcgtgatccgaaaggtgacccggatctggggcgtgatccgaaaggtgacccggaa

SEQ ID NO: 6 - RNA, 41 nt, unidentified
  source 1..41: mol_type = other RNA
tacccccggagtgttttatacgcagagatggagacaacaag

SEQ ID NO: 7 - RNA, 22 nt, unidentified
  source 1..22: mol_type = other RNA
atgggcatataaagaagtatgt

SEQ ID NO: 8 - RNA, 22 nt, unidentified
  source 1..22: mol_type = other RNA
atgggcatataaagaagtatgt

SEQ ID NO: 9 - RNA, 21 nt, unidentified
  source 1..21: mol_type = other RNA
gccaatatttctgtgctgcta

SEQ ID NO: 10 - RNA, 21 nt, unidentified
  source 1..21: mol_type = other RNA
gccaatatttctgtgctgcta

SEQ ID NO: 11 - RNA, 22 nt, unidentified
  source 1..22: mol_type = other RNA
ccaaacactgctgggtaagacg

SEQ ID NO: 12 - RNA, 23 nt, unidentified
  source 1..23: mol_type = other RNA
tccatcattacccggcagtatta

SEQ ID NO: 13 - RNA, 22 nt, unidentified
  source 1..22: mol_type = other RNA
gtataacccgggccaaaagctc

SEQ ID NO: 14 - RNA, 22 nt, unidentified
  source 1..22: mol_type = other RNA
cctggccgtgtggttagtgatt

SEQ ID NO: 15 - RNA, 22 nt, unidentified
  source 1..22: mol_type = other RNA
aactatacaatctactacctca

SEQ ID NO: 16 - RNA, 22 nt, unidentified
  source 1..22: mol_type = other RNA
gggaaggcaatagattgtatag

SEQ ID NO: 17 - RNA, 22 nt, unidentified
  source 1..22: mol_type = other RNA
accagagatgcagcactgcacc

SEQ ID NO: 18 - RNA, 21 nt, unidentified
  source 1..21: mol_type = other RNA
gagctacagtgcttcatctca

SEQ ID NO: 19 - RNA, 22 nt, unidentified
  source 1..22: mol_type = other RNA
aggatctacactggctactgag

SEQ ID NO: 20 - RNA, 21 nt, unidentified
  source 1..21: mol_type = other RNA
acccagtagccagatgtagct

SEQ ID NO: 21 - RNA, 21 nt, unidentified
  source 1..21: mol_type = other RNA
agtagtgctttctactttatg

SEQ ID NO: 22 - RNA, 23 nt, unidentified
  source 1..23: mol_type = other RNA
tccataaagtaggaaacactaca

SEQ ID NO: 23 - RNA, 22 nt, unidentified
  source 1..22: mol_type = other RNA
caaacaccattgtcacactcca

SEQ ID NO: 24 - RNA, 22 nt, unidentified
  source 1..22: mol_type = other RNA
tatttagtgtgataatggcgtt

SEQ ID NO: 25 - RNA, 22 nt, unidentified
  source 1..22: mol_type = other RNA
aacccatggaattcagttctca

SEQ ID NO: 26 - RNA, 22 nt, unidentified
  source 1..22: mol_type = other RNA
ctgaagaactgaatttcagagg

SEQ ID NO: 27 - RNA, 23 nt, unidentified
  source 1..23: mol_type = other RNA
gcaatcagctaactacactgcct

SEQ ID NO: 28 - RNA, 22 nt, unidentified
  source 1..22: mol_type = other RNA
cctggccgtgtggttagtgatt

SEQ ID NO: 29 - RNA, 23 nt, unidentified
  source 1..23: mol_type = other RNA
ctacctgcactgtaagcactttg

SEQ ID NO: 30 - RNA, 22 nt, unidentified
  source 1..22: mol_type = other RNA
ctacaagtgccttcactgcagt

SEQ ID NO: 31 - RNA, 22 nt, unidentified
  source 1..22: mol_type = other RNA
tcacaagttagggtctcaggga

SEQ ID NO: 32 - RNA, 22 nt, unidentified
  source 1..22: mol_type = other RNA
agctcccaagagcctaacccgt

SEQ ID NO: 33 - RNA, 22 nt, unidentified
  source 1..22: mol_type = other RNA
agcctatcctggattacttgaa

SEQ ID NO: 34 - RNA, 22 nt, unidentified
  source 1..22: mol_type = other RNA
gaaacaagtaatcaagaatagg

SEQ ID NO: 35 - RNA, 23 nt, unidentified
  source 1..23: mol_type = other RNA
agcattgcaaccgatcccaacct

SEQ ID NO: 36 - RNA, 22 nt, unidentified
  source 1..22: mol_type = other RNA
acaggccgggacaagtgcaata

SEQ ID NO: 37 - RNA, 23 nt, unidentified
  source 1..23: mol_type = other RNA
ctacctgcactataagcacttta

SEQ ID NO: 38 - RNA, 22 nt, unidentified
  source 1..22: mol_type = other RNA
ctttaagtgctcataatgcagt

SEQ ID NO: 39 - RNA, 22 nt, unidentified
  source 1..22: mol_type = other RNA
ctcggggcagctcagtacagga

SEQ ID NO: 40 - RNA, 21 nt, unidentified
  source 1..21: mol_type = other RNA
atcctgtactgagctgccccg

SEQ ID NO: 41 - DNA, 22 bp, unidentified
  source 1..22: mol_type = other DNA
acaggattcaaaccaggactgt

SEQ ID NO: 42 - DNA, 20 bp, unidentified
  source 1..20: mol_type = other DNA
tgggcatctttccttccgaa

SEQ ID NO: 43 - DNA, 22 bp, unidentified
  source 1..22: mol_type = other DNA
atactcaccgaacggaagaacc

SEQ ID NO: 44 - DNA, 22 bp, unidentified
  source 1..22: mol_type = other DNA
ccattagcccattcaccacctc

SEQ ID NO: 45 - DNA, 21 bp, unidentified
  source 1..21: mol_type = other DNA
aggtcggtgtgaacggatttg

SEQ ID NO: 46 - DNA, 23 bp, unidentified
  source 1..23: mol_type = other DNA
tgtagaccatgtagttgaggtca

SEQ ID NO: 47 - RNA, 1653 nt, unidentified
  source 1..1653: mol_type = other RNA
atggccgatgctaagaacattaagaagggccctgctcccttctaccctctggaggatggcaccgctggcgagcagctgcacaaggccatgaagaggtatgccctggtgcctggcaccattgccttcaccgatgcccacattgaggtggacatcacctatgccgagtacttcgagatgtctgtgcgcctggccgaggccatgaagaggtacggcctgaacaccaaccaccgcatcgtggtgtgctctgagaactctctgcagttcttcatgccagtgctgggcgccctgttcatcggagtggccgtggcccctgctaacgacatttacaacgagcgcgagctgctgaacagcatgggcatttctcagcctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgacttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgatttgcggctttcgggtggtgctgatgtaccgcttcgaggaggagctgttcctgcgcagcctgcaagactacaaaattcagtctgccctgctggtgccaaccctgttcagcttcttcgctaagagcaccctgatcgacaagtacgacctgtctaacctgcacgagattgcctctggcggcgccccactgtctaaggaggtgggcgaagccgtggccaagcgctttcatctgccaggcatccgccagggctacggcctgaccgagacaaccagcgccattctgattaccccagagggcgacgacaagcctggcgccgtgggcaaggtggtgccattcttcgaggccaaggtggtggacctggacaccggcaagaccctgggagtgaaccagcgcggcgagctgtgtgtgcgcggccctatgattatgtccggctacgtgaataaccctgaggccacaaacgccctgatcgacaaggacggctggctgcactctggcgacattgcctactgggacgaggacgagcacttcttcatcgtggaccgcctgaagtctctgatcaagtacaagggctaccaggtggccccagccgagctggagtctatcctgctgcagcaccctaacattttcgacgccggagtggccggcctgcccgacgacgatgccggcgagctgcctgccgccgtcgtcgtgctggaacacggcaagaccatgaccgagaaggagatcgtggactatgtggccagccaggtgacaaccgccaagaagctgcgcggcggagtggtgttcgtggacgaggtgcccaagggcctgaccggcaagctggacgcccgcaagatccgcgagatcctgatcaaggctaagaaaggcggcaagatcgccgtgtaa

SEQ ID NO: 48 - RNA, 1188 nt, unidentified
  source 1..1188: mol_type = other RNA
atgccgggccggagttgcgtcgccttagtcctcctggctgccgccgtcagctgtgccgtcgcgcagcacgcgccgccgtggacagaggactgcagaaaatcaacctatcctccttcaggaccaacgtacagaggtgcagttccatggtacaccataaatcttgacttaccaccctacaaaagatggcatgaattgatgcttgacaaggcaccagtgctaaaggttatagtgaattctctgaagaatatgataaatacattcgtgccaagtggaaaaattatgcaggtggtggatgaaaaattgcctggcctacttggcaactttcctggcccttttgaagaggaaatgaagggtattgccgctgttactgatatacctttaggagagattatttcattcaatattttttatgaattatttaccatttgtacttcaatagtagcagaagacaaaaaaggtcatctaatacatgggagaaacatggattttggagtatttcttgggtggaacataaataatgatacctgggtcataactgagcaactaaaacctttaacagtgaatttggatttccaaagaaacaacaaaactgtcttcaaggcttcaagctttgctggctatgtgggcatgttaacaggattcaaaccaggactgttcagtcttacactgaatgaacgtttcagtataaatggtggttatctgggtattctagaatggattctgggaaagaaagatgtcatgtggatagggttcctcactagaacagttctggaaaatagcacaagttatgaagaagccaagaatttattgaccaagaccaagatattggccccagcctactttatcctgggaggcaaccagtctggggaaggttgtgtgattacacgagacagaaaggaatcattggatgtatatgaactcgatgctaagcagggtagatggtatgtggtacaaacaaattatgaccgttggaaacatcccttcttccttgatgatcgcagaacgcctgcaaagatgtgtctgaaccgcaccagccaagagaatatctcatttgaaaccatgtatgatgtcctgtcaacaaaacctgtcctcaacaagctgaccgtatacacaaccttgatagatgttaccaaaggtcaattcgaaacttacctgcgggactgccctgacccttgtataggttggtga

SEQ ID NO: 49 - RNA, 1032 nt, unidentified
  source 1..1032: mol_type = other RNA
atgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctagaagatggcgattag

SEQ ID NO: 50 - RNA, 564 nt, unidentified; identical to SEQ ID NO: 1
  source 1..564: mol_type = other RNA

SEQ ID NO: 51 - RNA, 760 nt, unidentified
  source 1..760: mol_type = other RNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagaagcggcccgcagcaaccaagaaggcaggacaggccaagaagaagaagtaag

SEQ ID NO: 52 - RNA, 801 nt, unidentified
  source 1..801: mol_type = other RNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggagatccaaaaaagaagagaaaggtaggcgatccaaaaaagaagagaaaggtaggtgatccaaaaaagaagagaaaggtataa

SEQ ID NO: 53 - RNA, 1401 nt, unidentified
  source 1..1401: mol_type = other RNA
atggagtttggtctttcctgggtattcctggtggcccttttgcgcggcgtacaatgtgaagcaaagctccaagagagtggtcccgtattggtgaaacctggagcatctgtcaagatgagttgtaaagccagcggatacactttcaccgattattatatgaactgggttaaacaaagccacggcaaaagtctggaatggataggggtaatcaatccatataacggcgatacctcctacaaccaaaaattcaagggtaaggctactttgacagttgataagtcctcttccaccgcatacatggagctcaactcactgacttcagaagacagtgctgtctactattgtgctaggtattacggatcatggtttgcttactgggggcagggtactttgatcaccgttagtgctgccaaaactacagccccctccgtgtaccctctcgcacccgtttgcggtgacacaactggctcctctgtaaccctgggctgcctggtaaagggctatttcccagagcccgtcactctgacttggaactctgggtctctgagttcaggtgtacacacctttcctgccgtacttcaaagcgatctctacacacttagctctagtgtaacagtaacatcctctacctggccatcacagtctatcacatgcaacgtagcacaccctgcctctagcacaaaggttgacaagaaaatagagcccagaggaccaaccatcaagccttgtcccccatgcaaatgtcctgcccccaatcttttgggcggtccctccgtcttcatcttcccccctaaaattaaggacgttctcatgatttccctgtctccaatcgtcacctgcgtcgtagtggatgtttctgaagacgacccagatgtgcaaataagttggttcgtcaataatgtcgaggtgcatacagcccaaactcaaacacatagagaggactacaattcaacccttagggttgttagcgcacttcccatacagcaccaggactggatgagtggtaaagaattcaagtgcaaagtgaacaataaggaccttcccgctcctattgagcgcacaatcagtaagcctaaaggttctgtccgggcacctcaagtttacgtcctcccacccccagaggaagagatgaccaaaaagcaagtgactctgacttgtatggtaacagactttatgcccgaggatatttatgttgaatggactaacaacggaaagactgagctgaactataagaatacagaacccgtcttggactcagacgggagttatttcatgtattccaaattgagagtagaaaaaaaaaattgggtagagcgcaactcttatagttgcagtgttgtacatgaaggacttcacaatcaccataccacaaagagtttttctcggacaccagggaaatag

SEQ ID NO: 54 - RNA, 717 nt, unidentified
  source 1..717: mol_type = other RNA
atgcttccctctcagttgatcggcttccttctgctttgggttcccgcaagccgcggtgacattgtgatgacccagactacactgtctctccctgtatccttgggggaccaagcctccattagctgtagatcttcacaatccattgtccattccaatgggaatacatatctggagtggtatttgcaaaagcctggtcagtctcccaaactgctgatatataaagtgtctaaccgattcagtggggtgccagataggtttagcgggagcggttcaggaactgactttacacttaagatttcaagagttgaagccgaggatcttggtgtttactattgctttcagggatctcatgtgccatacacttttggagggggcactaaactcgagattaaaagggcagacgctgcccctacagttagtatattcccaccatcaagcgaacagttgacttcaggaggggcatccgtagtatgttttcttaacaatttttacccaaaagacattaacgtcaagtggaagatcgatgggtcagaacgacagaatggagttttgaatagttggactgaccaagactccaaagattccacttactccatgtcctcaaccctgaccttgaccaaggatgagtacgaaagacacaactcctatacctgcgaagccactcacaagacaagcaccagccccatagttaagtcttttaatcggaacgagtgctag

SEQ ID NO: 55 - RNA, 1266 nt, unidentified
  source 1..1266: mol_type = other RNA
atggcgtcctcctccgtccctcccgccaccgcacccgcggcagctggaggccccggcccgggattcggcttcgcctccaaaaccaagaagaagcatttcgtacagcagaaagtgaaggtgttccgggccgcggacccgctggtgggcgtgttcctgtggggcgtcgctcactcgatcaatgagctcagccaggtaccgcccccagtgatgttgctgccagatgactttaaagccagctccaagatcaaggtcaacaatcactttttccatagagaaaatcttcccagtcatttcaagttcaaggagtattgtccccaggtcttcaggaacctgagagatcggtttgccatcgatgatcacgattacttggtgtcccttactcgaagccccccaagcgaaactgaaggcagtgatggccgtttccttatctcctatgaccgcactctggtcatcaaggaagtgtcgagcgaagatattgcggatatgcacagcaacctctccaactaccaccagtacatagtgaaatgtcatggcaacacgcttctgccccagttcctgggcatgtaccgagtcagtgtagaaaatgaagacagctacatgctcgtgatgcgcaatatgtttagtcatcgtcttcctgtgcataggaagtatgacctcaagggctctctagtgtcccgggaagccagcgataaggaaaaggttaaagaactgccaacactaaaggatatggactttcttaacaagaaccagaaagtgtatattggtgaagaagaaaagaaagtattcctggagaagctgaagcgagatgtggagtttctagtgcagctgaagatcatggactacagcctcctgttgggcatccacgacatcatccggggctctgaacccgaggaagaggggcctgtgagggaggaggagtctgagtgggatggggactgtaacctggctggacctcccgccctggtgggctcctatggtacctcccctgagggtatcggaggctacatccattcccaccggccactgggcccaggagagtttgagtccttcatcgatgtctatgctatccggagtgcggagggggccccccagaaggaggtgtatttcatgggcctcattgacattctgacacagtatgatgccaagaagaaagcagctcatgcagccaagactgtcaagcacggggcgggggcagagatctccactgtccatcctgagcagtatgctaagcgattcctggactttattgccaacatctttgcctaa

SEQ ID NO: 56 - RNA, 1182 nt, unidentified
  source 1..1182: mol_type = other RNA
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactga